(19) United States
(12) Patent Application Publication
(10) Pub. No.: US 2023/0348421 A1
(43) Pub. Date: Nov. 2, 2023

(54) 5-HT2A RECEPTOR INHIBITOR OR INVERSE AGONIST, PREPARATION METHOD THEREFOR, AND APPLICATION THEREOF

(71) Applicant: GENEORA PHARMA (SHIJIAZHUANG) CO., LTD., Shijiazhuang, Hebei (CN)

(72) Inventors: Rui ZHANG, Yantai, Shandong (CN); Liang YE, Yantai, Shandong (CN); Mingxu MA, Yantai, Shandong (CN); Wenyan WANG, Yantai, Shandong (CN); Yusen DAI, Yantai, Shandong (CN); Jingwei TIAN, Yantai, Shandong (CN)

(21) Appl. No.: 18/005,952

(22) PCT Filed: Jul. 22, 2021
(86) PCT No.: PCT/CN2021/107774

§ 371 Date: Jan. 18, 2023

(30) Foreign Application Priority Data
Jul. 22, 2020 (CN) 202010708352.8
Apr. 3, 2021 (CN) 202110364023.0

(51) Int. Cl. C07D 401/12; C07D 409/12; C07D 401/06; C07D 405/14; C07D 405/12; C07D 211/58; C07D 487/08; C07C 275/26

(52) U.S. Cl. CPC C07D 401/12; C07D 409/12; C07D 401/06; C07D 405/14; C07D 405/12; C07D 211/58; C07D 487/08; C07C 275/26

(57) ABSTRACT

The present invention relates to a novel compound as a 5-HT_{2A} receptor inhibitor or inverse agonist, a preparation method therefor, and a pharmaceutical composition thereof. The present invention also relates to an application of the compound or the pharmaceutical composition in the preparation of a drug for treating 5-HT_{2A} receptor-related diseases, the diseases comprising: non-motor symptoms caused by Parkinson's disease: delusion, illusion, depression, anxiety, cognitive disorder, and sleep disorder; dementia-related mental diseases; major depressive disorder; or negative symptoms of schizophrenia, etc.

### TECHNICAL FIELD

The present invention relates to a class of compounds as selective 5-hydroxytryptamine 2A (5-HT_{2A}) receptor inhibitors or inverse agonists, a preparation method therefor, and the use thereof in the field of 5-HT_{2A} receptor-related diseases.

### BACKGROUND ART

Parkinson's disease (PD) is a common neurodegenerative disease with an average age of onset of around 60 years (Degirmenci, Yildiz. Cumhuriyet Medical Journal (2017), 39 (3), 509-517). According to data from the National Institutes of Health (NIH) in 2018, there are about 4 million to 6 million patients with Parkinson's disease worldwide. Among them, up to 50% of patients with Parkinson's disease develop severe symptoms of illusion or delusion in the course of their illness, which seriously affects the quality of life of patients and leads to high morbidity and mortality.

In 2016, pimavanserin comes into the market for treating illusion and delusion symptoms accompanying Parkinson's disease as approved by the U.S. FDA, and becomes the first approved drug for treating such indications.

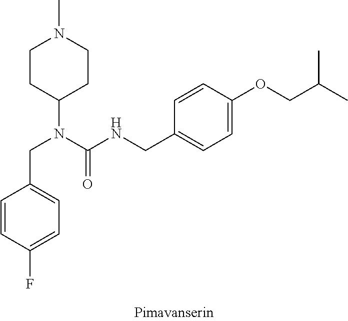 [SMILES: CC(C)COc1ccc(CNC(=O)N(Cc2ccc(F)cc2)C2CCN(C)CC2)cc1]

Pimavanserin acts on a 5-HT_{2A} receptor which is a main excitatory receptor subtype in the 5-HT receptor family and is a ligand-gated channel receptor and a G protein-coupled receptor. The 5-HT_{2A} receptor has a function closely related to neuronal excitation, behavioral effects, learning and memory, anxiety, etc., and thus is an important target for antipsychotic drugs and schizophrenia treatment (Price, D. L., et al. Behavioral Pharmacology (2012), 23(4), 426-433). The first-generation antipsychotic drug is mainly used to inhibit a dopamine D₂ receptor and thus has serious extrapyramidal side effects. The second-generation antipsychotic drug, in addition to inhibiting the D₂ receptor, has more inhibitory effects on a specific 5-HT receptor, particularly the 5-HT_{2A} receptor, and has higher safety, i.e., fewer extrapyramidal side effects in comparison with the first-generation antipsychotic drug. However, the second-generation antipsychotic drug still has inhibitory activities against the D₂ receptor and thus still has extrapyramidal side effects. Moreover, such drug has the side effect of weight gain in different degrees.

Therefore, developing a selective 5-HT_{2A} receptor inhibitor or inverse agonist can eliminate the extrapyramidal side effects related to dopamine receptor inhibition and the side effect of weight gain of the first-generation and second-generation antipsychotic drugs. The receptor inhibitor or inverse agonist has higher safety, and can also be applied to the treatment of other diseases related to 5-HT_{2A} receptors to meet the needs of clinical patients.

### SUMMARY OF THE INVENTION

In one aspect, the present invention provides a compound of formula (I) or a pharmaceutically acceptable salt thereof,

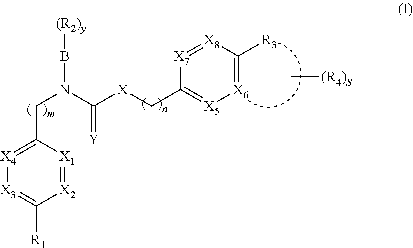

wherein
at least one of X₁ and X₄ is N, and the other one is optionally CR₁ or N;
X₂ and X₃ are each independently selected from CR₁ and N;
X₅ is independently selected from CR₃ₐ or N;
X₆ is independently selected from CR_{3b} or N;
X₇ is independently selected from CR_{3c} or N;
X₈ is independently selected from CR_{3d} or N;
group B is linear or branched C₁₋₆ alkyl or a 5-6-membered nitrogen heterocyclic group, and the linear or branched C₁₋₆ alkyl or the 5-6-membered nitrogen heterocyclic group is optionally substituted with one or more deuterium atoms;
each R₁ is the same or different and is independently selected from a hydrogen atom, linear or branched C₁₋₁₀ alkyl or halogen;
each R₂ is the same or different and is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3b}, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy; or when X₆ is CR_{3b}, X₆ and R₃ together with the atoms to which they are attached form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy;
X is selected from —NH— or —(CH₂)₁₋₄NH—;
Y is selected from O or S;
m and n are independently selected from 0, 1, 2 and 3;
s is independently selected from 1, 2, 3, 4, 5 and 6; and
y is independently selected from 0, 1, 2, 3, 4 and 5.

In one embodiment of the compound of formula (I), at least one of X₁ and X₄ is N, and the other one is optionally CR₁; and X₂ and X₃ are each independently selected from CR₁, preferably CH.

In one embodiment of the compound of formula (I), X₁ and X₄ are both N, and X₂ and X₃ are each independently selected from CR₁.

In one embodiment of the compound of formula (I), at least one of X₁ and X₄ is N, and the other one is optionally CR₁; and at least one of X₂ and X₃ is N, and the other one is optionally CR₁.

In one embodiment of the compound of formula (I), X₁ and X₄ are both N; and at least one of X₂ and X₃ is N, and the other one is optionally CR₁.

In one embodiment of the compound of formula (I), at least one of X₁ and X₄ is N, and the other one is optionally CR₁; and X₂ and X₃ are both N.

In one embodiment of the compound of formula (I), X₁ and X₄ are both N, and X₂ and X₃ are both N.

In one embodiment of the compound of formula (I), X₁ is preferably N, and X₂, X₃ and X₄ are all CR₁.

In one embodiment of the compound of formula (I), group B is —CH₂—, —(CH₂)₂—, —(CH₂)₃—, —(CH₂)₄—, -CD₂-, -(CD₂)₂-, -(CD₂)₃- or -(CD₂)₄-, and R₂ is independently selected from dimethylamine or diethylamine, wherein the dimethylamine or the diethylamine is optionally substituted with one or more deuterium atoms;
or group B is selected from piperidinyl, wherein the piperidinyl is optionally substituted with one or more deuterium atoms, and R₂ is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms.

In one embodiment of the compound of formula (I), each R₁ is the same or different and is independently selected from a hydrogen atom, F, Cl, Br, I, methyl, ethyl, propyl, isopropyl, butyl, isobutyl, sec-butyl or tert-butyl.

In one embodiment of the compound of formula (I), each R₂ is the same or different and is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms.

In one embodiment of the compound of formula (I), R₃, R₃ₐ, R_{3b}, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—.

In one embodiment of the compound of formula (I), when X₆ is CR_{3b}, X₆ and R₃ together with the atoms to which they are attached form a ring system and are optionally substituted with one or more of the same or different R₄, wherein the ring system is preferably selected from dihydrofuran, dihydropyrrole or dihydrothiophene, and wherein each R₄ is the same or different and is independently selected from a hydrogen atom, methyl, ethyl, propyl, isopropyl, butyl, isobutyl, sec-butyl or tert-butyl.

In one specific embodiment of the present invention, any one of the above-mentioned compounds of formula (I) may be a deuterated analog thereof. The deuterated analog refers to an analog formed by substituting one or more hydrogen atoms of a compound with deuterium atoms.

Compared with pimavanserin, the compound of formula (I) provided by the present invention has higher 5-HT_{2A} antagonistic activity and 5-HT_{2A} inverse agonistic activity, and/or lower cardiotoxicity. Particularly, when X₁ and/or X₄ are/is N, and X₂ and X₃ are CH, the 5-HT_{2A} antagonistic activity and/or the 5-HT_{2A} inverse agonistic activity can be further improved.

In another aspect, the present invention provides a compound of formula (II) or a pharmaceutically acceptable salt thereof,

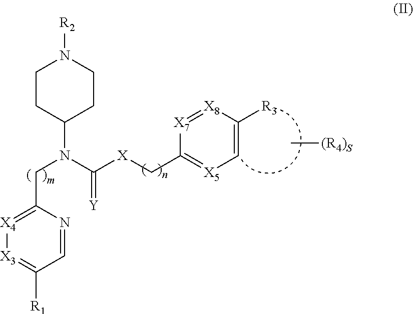

wherein
X₃ and X₄ are each independently selected from CR₁ and N;
X₅ is independently selected from CR₃ₐ or N;
X₇ is independently selected from CR_{3c} or N;
X₈ is independently selected from CR_{3d} or N;
each R₁ is the same or different and is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy;
X is selected from —NH— or —(CH₂)₁₋₄NH—;
Y is selected from O or S;
m and n are independently selected from 0, 1, 2 and 3; and
s is independently selected from 1, 2, 3, 4, 5 and 6.

In one embodiment of the compound of formula (II), X₃ and X₄ are both CR₁.

In one embodiment of the compound of formula (II), X₃ is N and X₄ is CR₁.

In one embodiment of the compound of formula (II), X₃ is CR₁ and X₄ is N.

In one embodiment of the compound of formula (II), each R₁ is the same or different and is independently selected from a hydrogen atom, F, Cl, Br, I, methyl, ethyl, propyl, isopropyl, butyl, isobutyl, sec-butyl or tert-butyl.

In one embodiment of the compound of formula (II), each R₂ is the same or different and is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms, preferably a hydrogen atom, a deuterium atom, methyl, ethyl, deuterated methyl or deuterated ethyl, and more preferably methyl or deuterated methyl.

In one embodiment of the compound of formula (II), R₃, R₃ₐ, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—.

In one embodiment of the compound of formula (II), R₃ and the carbon atom to which it is attached together with adjacent carbon atoms form a ring system and are optionally substituted with one or more of the same or different R₄, wherein the ring system is preferably selected from dihydrofuran, dihydropyrrole or dihydrothiophene, and wherein each R₄ is the same or different and is independently selected from a hydrogen atom, methyl, ethyl, propyl, isopropyl, butyl, isobutyl, sec-butyl or tert-butyl.

In one specific embodiment of the present invention, any one of the above-mentioned compounds of formula (II) may be a deuterated analog thereof. The deuterated analog refers to an analog formed by substituting one or more hydrogen atoms of a compound with deuterium atoms.

Compared with pimavanserin, the compound of formula (II) provided by the present invention has higher 5-HT_{2A} antagonistic activity and 5-HT_{2A} inverse agonistic activity, and/or lower cardiotoxicity. In another aspect, the present invention provides a compound of formula (III) or a pharmaceutically acceptable salt thereof,

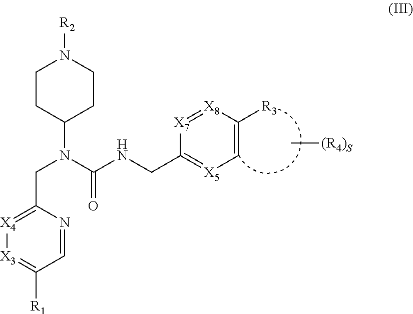

wherein
X₃ and X₄ are each independently selected from CR₁ and N;
X₅ is independently selected from CR₃ₐ or N;
X₇ is independently selected from CR_{3c} or N;
X₈ is independently selected from CR_{3d} or N;
each R₁ is the same or different and is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy; and
s is independently selected from 1, 2, 3, 4, 5 and 6.

In one embodiment of the compound of formula (III), X₃ and X₄ are both CR₁.

In one embodiment of the compound of formula (III), X₃ is N and X₄ is CR₁.

In one embodiment of the compound of formula (III), X₃ is CR₁ and X₄ is N.

In one embodiment of the compound of formula (III), each R₁ is the same or different and is independently selected from a hydrogen atom, F, Cl, Br, I, methyl, ethyl, propyl, isopropyl, butyl, isobutyl, sec-butyl or tert-butyl.

In one embodiment of the compound of formula (III), each R₂ is the same or different and is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms, preferably a hydrogen atom, a deuterium atom, methyl, ethyl, deuterated methyl or deuterated ethyl, and more preferably methyl or deuterated methyl.

In one embodiment of the compound of formula (III), R₃, R₃ₐ, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—.

In one embodiment of the compound of formula (III), R₃ and the carbon atom to which it is attached together with adjacent carbon atoms form a ring system and are optionally substituted with one or more of the same or different R₄,
wherein the ring system is preferably selected from dihydrofuran, dihydropyrrole or dihydrothiophene, and
wherein each R₄ is the same or different and is independently selected from a hydrogen atom, methyl, ethyl, propyl, isopropyl, butyl, isobutyl, sec-butyl or tert-butyl.

In one specific embodiment of the present invention, any one of the above-mentioned compounds of formula (III) may be a deuterated analog thereof. The deuterated analog refers to an analog formed by substituting one or more hydrogen atoms of a compound with deuterium atoms.

Compared with pimavanserin, the compound of formula (III) provided by the present invention has higher 5-HT_{2A} antagonistic activity and 5-HT_{2A} inverse agonistic activity, and/or lower cardiotoxicity. In another aspect, the present invention provides a compound of formula (IV) or a pharmaceutically acceptable salt thereof,

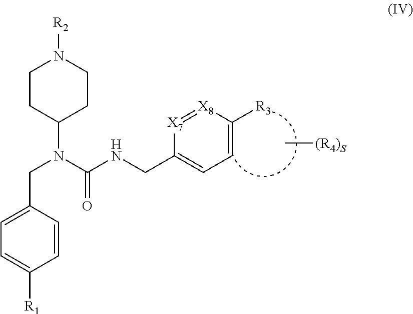

wherein
X₇ is independently selected from CR_{3c} or N;
X₈ is independently selected from CR_{3d} or N;
R₁ is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy; and
s is independently selected from 1, 2, 3, 4, 5 and 6.

In one embodiment of the compound of formula (IV), each R₁ is the same or different and is independently selected from a hydrogen atom, F, Cl, Br, I, methyl, ethyl, propyl, isopropyl, butyl, isobutyl, sec-butyl or tert-butyl.

In one embodiment of the compound of formula (IV), each R₂ is the same or different and is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms.

In one embodiment of the compound of formula (IV), R₃, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—.

In one embodiment of the compound of formula (IV), R₃ and the carbon atom to which it is attached together with adjacent carbon atoms form a ring system and are optionally substituted with one or more of the same or different R₄,
wherein the ring system is preferably selected from dihydrofuran, dihydropyrrole or dihydrothiophene, and
wherein each R₄ is the same or different and is independently selected from a hydrogen atom, methyl, ethyl, propyl, isopropyl, butyl, isobutyl, sec-butyl or tert-butyl.

In one specific embodiment of the present invention, any one of the above-mentioned compounds of formula (IV) may be a deuterated analog thereof. The deuterated analog refers to an analog formed by substituting one or more hydrogen atoms of a compound with deuterium atoms.

Compared with pimavanserin, the compound of formula (IV) provided by the present invention has higher 5-HT_{2A} antagonistic activity and 5-HT_{2A} inverse agonistic activity, and/or lower cardiotoxicity.

In another aspect, the present invention provides a compound of formula (V) or a pharmaceutically acceptable salt thereof,

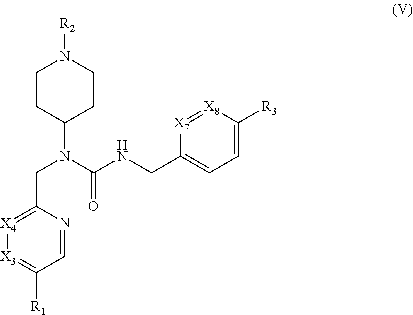

wherein
X₃ is independently selected from CR₅ or N;
X₄ is independently selected from CR₆ or N;
X₇ is independently selected from CR_{3c} or N;
X₈ is independently selected from CR_{3d} or N;
R₁, R₅ and R₆ are the same or different and are each independently selected from a hydrogen atom, linear or branched C₁₋₁₀ alkyl and halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms; and
R₃, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy.

In one embodiment of the compound of formula (V), X₃ and X₄ are CR₅ and CR₆, respectively.

In one embodiment of the compound of formula (V), X₃ is N and X₄ is CR₆.

In one embodiment of the compound of formula (V), X₃ is CR₅ and X₄ is N.

In one embodiment of the compound of formula (V), R₁, R₅ and R₆ are the same or different and are each independently selected from a hydrogen atom, F, Cl, Br, I, methyl, ethyl, propyl, isopropyl, butyl, isobutyl, sec-butyl and tert-butyl, preferably F, Cl, Br or I, and more preferably F.

In one embodiment of the compound of formula (V), R₂ is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms, preferably a hydrogen atom, a deuterium atom, methyl, ethyl, deuterated methyl or deuterated ethyl, and more preferably methyl or deuterated methyl.

In one embodiment of the compound of formula (V), R₃, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—.

In one embodiment of the compound of formula (V),
X₃ and X₇ are both CH;
X₄ is CR₆, wherein R₆ is a hydrogen atom or halogen, and X₄ is preferably CH or CF;
X₈ is CR_{3d}, and R₃ₐ is a hydrogen atom or halogen, preferably a hydrogen atom, F, C₁ or Br;
R₁ is halogen, preferably F;
R₂ is independently selected from a hydrogen atom, a deuterium atom or linear or branched C₁₋₅ alkyl, wherein the linear or branched C₁₋₅ alkyl is optionally substituted with one or more deuterium atoms, preferably a hydrogen atom, a deuterium atom, methyl, ethyl, propyl or isopropyl, wherein the methyl, the ethyl, the propyl or the isopropyl is optionally substituted with one or more deuterium atoms, preferably a hydrogen atom, methyl or ethyl, and more preferably methyl; R₃ is selected from hydroxyl, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy; R₃ is preferably substituted or unsubstituted linear or branched C₂₋₅ alkyl, substituted or unsubstituted linear or branched C₂₋₅ alkoxy, or substituted or unsubstituted linear or branched C₂₋₅ haloalkoxy; and R₃ is more preferably ethoxy, tert-butyl, isobutyloxy, 2-fluoroethoxy, 2,2-difluoroethoxy, 2,2,2-trifluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy or hydroxyl.

In one embodiment of the compound of formula (V),
X₃, X₄, X₇ and X₈ are all CH; R₁ is halogen, preferably F; R₂ is methyl; and R₃ is selected from linear or branched C₁₋₁₀ alkoxy substituted with halogen, preferably linear or branched C₂₋₅ haloalkoxy, and more preferably 2-fluoroethoxy, 2,2-difluoroethoxy, 2,2,2-trifluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy or 4,4,4-trifluorobutoxy.

In one specific embodiment of the present invention, any one of the above-mentioned compounds of formula (V) may be a deuterated analog thereof. The deuterated analog refers to an analog formed by substituting one or more hydrogen atoms of a compound with deuterium atoms.

Compared with pimavanserin, the compound of formula (V) provided by the present invention has higher 5-HT_{2A} antagonistic activity and 5-HT_{2A} inverse agonistic activity, and/or lower cardiotoxicity. Particularly, when R₃ is linear or branched C₁₋₁₀ alkoxy substituted with halogen, and X₃ and X₄ are CH, the 5-HT_{2A} antagonistic activity and/or the 5-HT_{2A} inverse agonistic activity can be further improved.

In another aspect, the present invention provides a compound of formula (VI) or a pharmaceutically acceptable salt thereof,

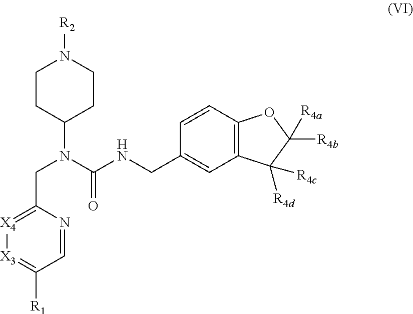

wherein
X₃ is independently selected from CR₅ or N;
X₄ is independently selected from CR₆ or N;
R₁, R₅ and R₆ are the same or different and are each independently selected from a hydrogen atom, linear or branched C₁₋₁₀ alkyl and halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms; and
R₄ₐ, R_{4b} and R_{4c} and R_{4d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy.

In one embodiment of the compound of formula (VI), X₃ and X₄ are CRs and CR₆, respectively.

In one embodiment of the compound of formula (VI), X₃ is N and X₄ is CR₆.

In one embodiment of the compound of formula (VI), X₃ is CR₅ and X₄ is N.

In one embodiment of the compound of formula (VI), R₁ is independently selected from a hydrogen atom, F, Cl, Br, I, methyl, ethyl, propyl, isopropyl, butyl, isobutyl, sec-butyl or tert-butyl, preferably F, Cl, Br or I, and more preferably F.

In one embodiment of the compound of formula (VI), R₂ is the same or different and is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms, preferably a hydrogen atom, a deuterium atom, methyl, ethyl, deuterated methyl or deuterated ethyl, and more preferably methyl or deuterated methyl.

In one embodiment of the compound of formula (VI), R₄ₐ, R_{4b}, R_{4c} and R_{4d} are the same or different and are each independently selected from a hydrogen atom, methyl, ethyl, propyl, isopropyl, butyl, isobutyl, sec-butyl and tert-butyl.

In one embodiment of the compound of formula (VI),
X₃ is CH;
X₄ is CR₆, wherein R₆ is a hydrogen atom or halogen, and X₄ is preferably CH or CF;
R₁ is halogen, preferably F;
R₂ is independently selected from a hydrogen atom, a deuterium atom or linear or branched C₁₋₅ alkyl, wherein the linear or branched C₁₋₅ alkyl is optionally substituted with one or more deuterium atoms, preferably a hydrogen atom, a deuterium atom, methyl, ethyl, propyl or isopropyl, wherein the methyl, the ethyl, the propyl or the isopropyl is optionally substituted with one or more deuterium atoms, preferably a hydrogen atom, a deuterium atom, methyl, ethyl, deuterated methyl or deuterated ethyl, and more preferably methyl or deuterated methyl;
R₄ₐ and R_{4b} are independently selected from a hydrogen atom, methyl and trifluoromethyl; and R_{4c} and R_{4d} are hydrogen atoms.

In one specific embodiment of the present invention, any one of the above-mentioned compounds of formula (VI) may be a deuterated analog thereof. The deuterated analog refers to an analog formed by substituting one or more hydrogen atoms of a compound with deuterium atoms.

Compared with pimavanserin, the compound of formula (VI) provided by the present invention has higher 5-HT_{2A} antagonistic activity and/or 5-HT_{2A} inverse agonistic activity, and lower cardiotoxicity.

In another aspect, the present invention provides compounds or pharmaceutically acceptable salts thereof or deuterated analogs thereof as shown below:

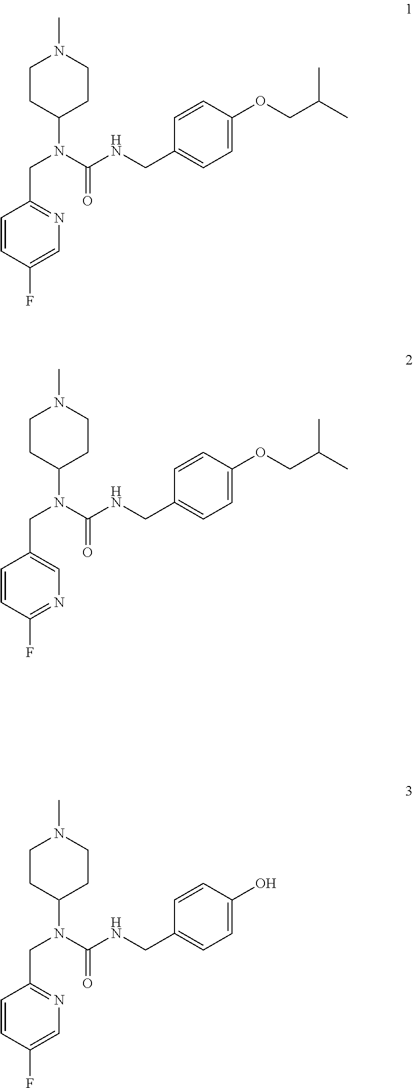 [SMILES: CC(C)COc1ccc(CNC(=O)N(Cc2ccc(F)cn2)C2CCN(C)CC2)cc1 ; CC(C)COc1ccc(CNC(=O)N(Cc2ccc(F)nc2)C2CCN(C)CC2)cc1 ; CN1CCC(N(Cc2ccc(F)cn2)C(=O)NCc2ccc(O)cc2)CC1]

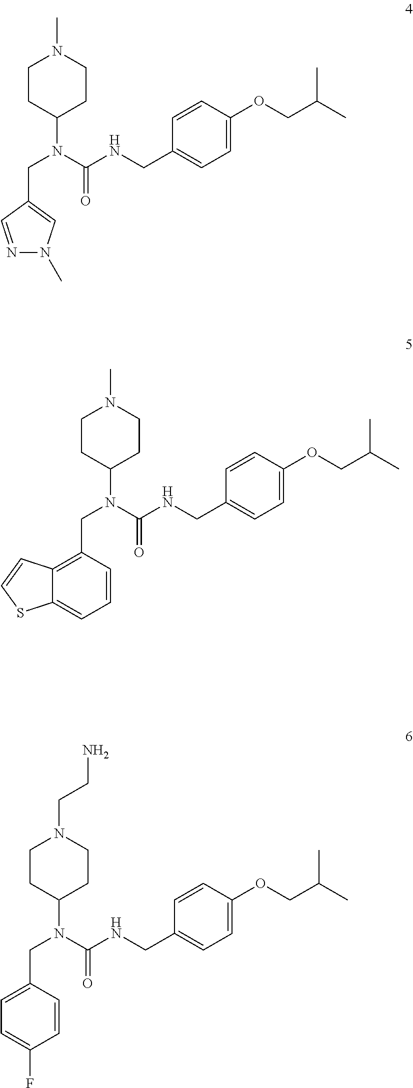 [SMILES: CC(C)COc1ccc(CNC(=O)N(Cc2cnn(C)c2)C2CCN(C)CC2)cc1 ; CC(C)COc1ccc(CNC(=O)N(Cc2cccc3sccc23)C2CCN(C)CC2)cc1 ; CC(C)COc1ccc(CNC(=O)N(Cc2ccc(F)cc2)C2CCN(CCN)CC2)cc1]

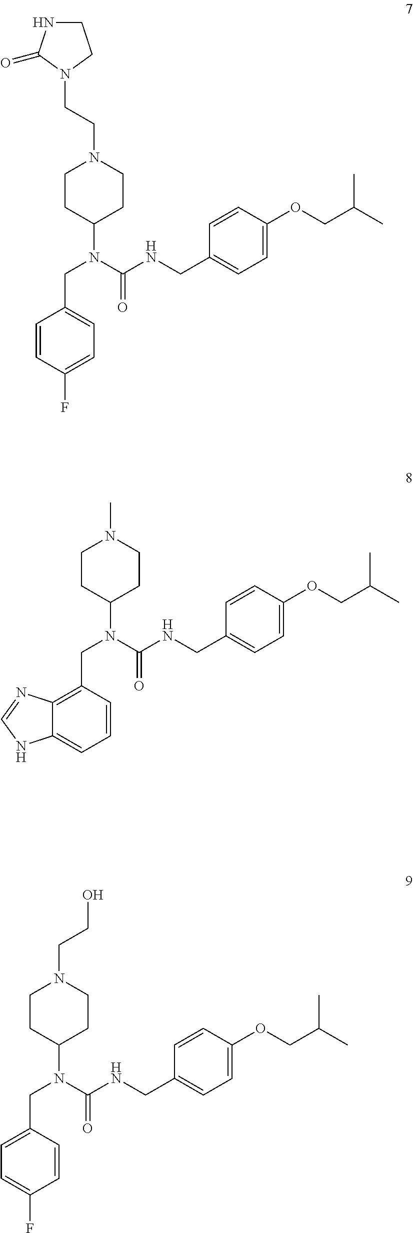 [SMILES: CC(C)COc1ccc(CNC(=O)N(Cc2ccc(F)cc2)C2CCN(CCN3CCNC3=O)CC2)cc1 ; CC(C)COc1ccc(CNC(=O)N(Cc2cccc3[nH]cnc23)C2CCN(C)CC2)cc1 ; CC(C)COc1ccc(CNC(=O)N(Cc2ccc(F)cc2)C2CCN(CCO)CC2)cc1]

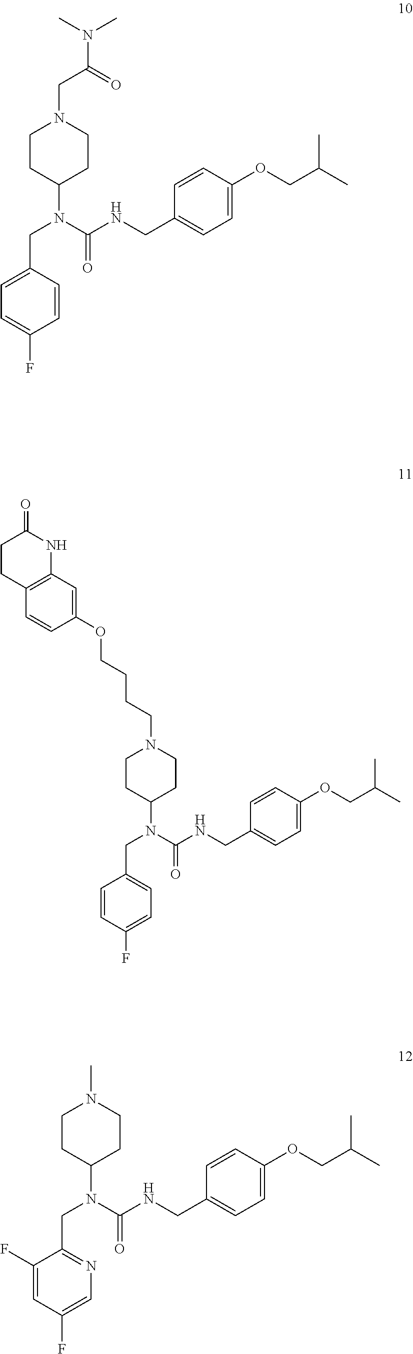 [SMILES: CC(C)COc1ccc(CNC(=O)N(Cc2ccc(F)cc2)C2CCN(CC(=O)N(C)C)CC2)cc1 ; CC(C)COc1ccc(CNC(=O)N(Cc2ccc(F)cc2)C2CCN(CCCCOc3ccc4c(c3)NC(=O)CC4)CC2)cc1 ; CC(C)COc1ccc(CNC(=O)N(Cc2ncc(F)cc2F)C2CCN(C)CC2)cc1]

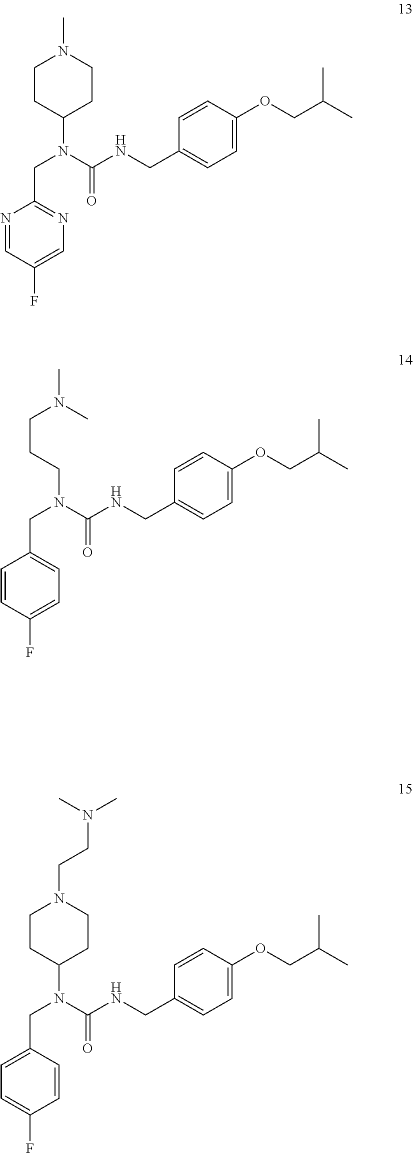 [SMILES: CC(C)COc1ccc(CNC(=O)N(Cc2ncc(F)cn2)C2CCN(C)CC2)cc1 ; CC(C)COc1ccc(CNC(=O)N(CCCN(C)C)Cc2ccc(F)cc2)cc1 ; CC(C)COc1ccc(CNC(=O)N(Cc2ccc(F)cc2)C2CCN(CCN(C)C)CC2)cc1]

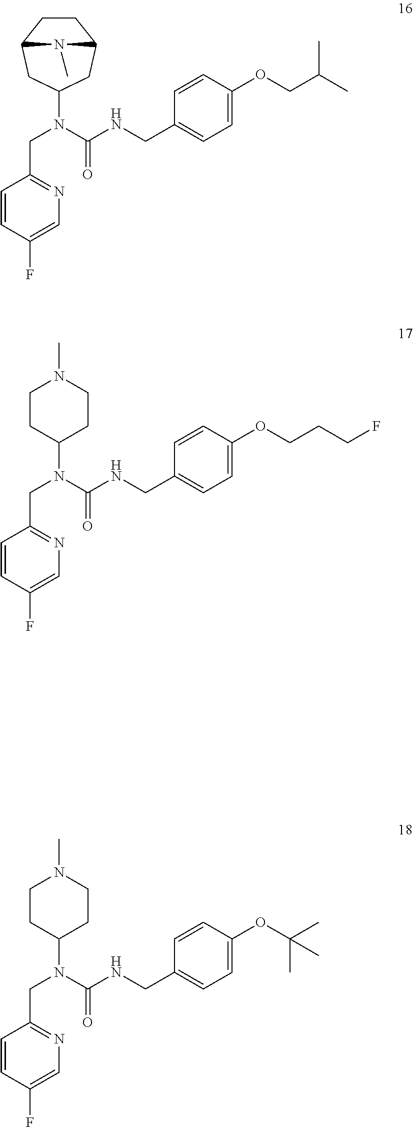

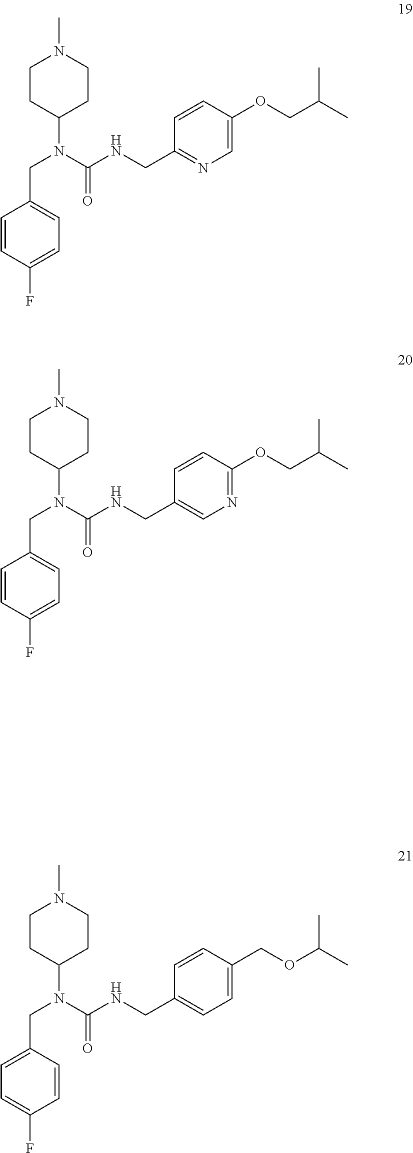 [SMILES: CC(C)COc1ccc(CNC(=O)N(Cc2ccc(F)cc2)C2CCN(C)CC2)nc1 ; CC(C)COc1ccc(CNC(=O)N(Cc2ccc(F)cc2)C2CCN(C)CC2)cn1 ; CC(C)OCc1ccc(CNC(=O)N(Cc2ccc(F)cc2)C2CCN(C)CC2)cc1]

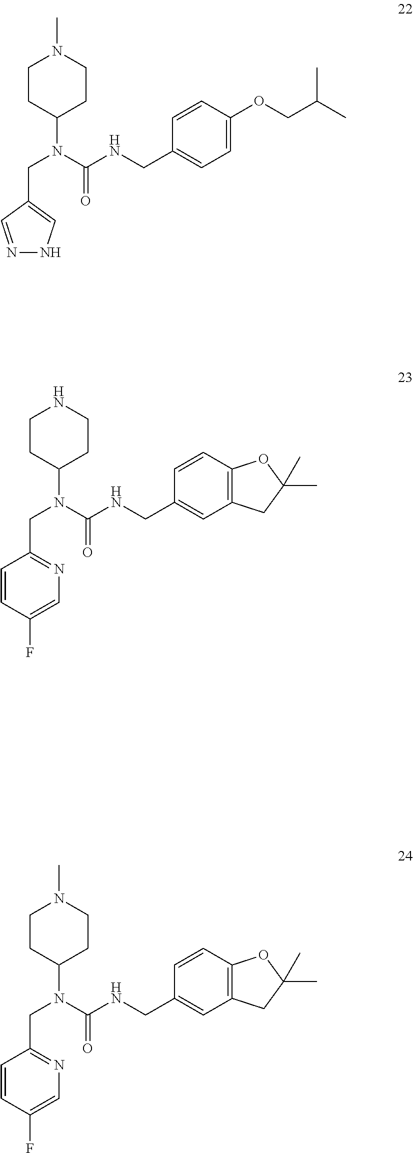 [SMILES: CC(C)COc1ccc(CNC(=O)N(Cc2cn[nH]c2)C2CCN(C)CC2)cc1 ; CC1(C)Cc2cc(CNC(=O)N(Cc3ccc(F)cn3)C3CCNCC3)ccc2O1 ; CN1CCC(N(Cc2ccc(F)cn2)C(=O)NCc2ccc3c(c2)CC(C)(C)O3)CC1]

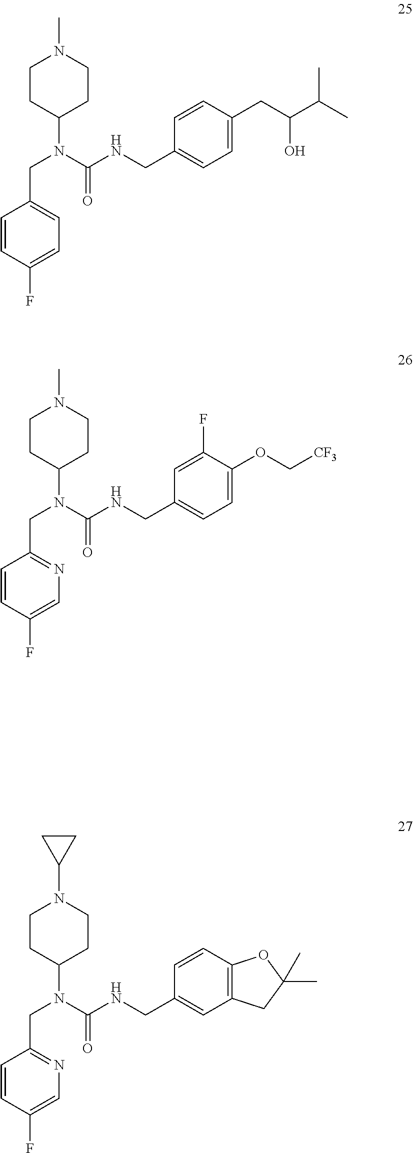 [SMILES: CC(C)C(O)Cc1ccc(CNC(=O)N(Cc2ccc(F)cc2)C2CCN(C)CC2)cc1 ; CN1CCC(N(Cc2ccc(F)cn2)C(=O)NCc2ccc(OCC(F)(F)F)c(F)c2)CC1 ; CC1(C)Cc2cc(CNC(=O)N(Cc3ccc(F)cn3)C3CCN(C4CC4)CC3)ccc2O1]

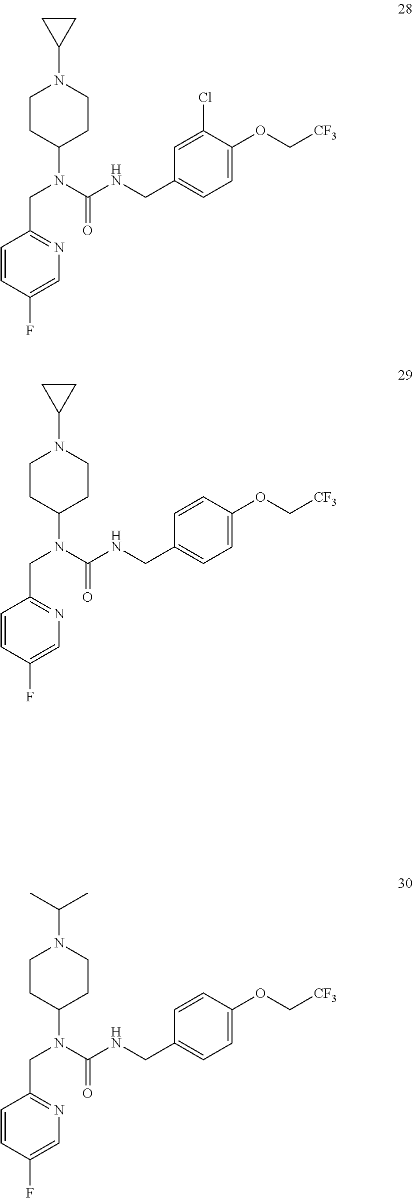 [SMILES: O=C(NCc1ccc(OCC(F)(F)F)c(Cl)c1)N(Cc1ccc(F)cn1)C1CCN(C2CC2)CC1 ; O=C(NCc1ccc(OCC(F)(F)F)cc1)N(Cc1ccc(F)cn1)C1CCN(C2CC2)CC1 ; CC(C)N1CCC(N(Cc2ccc(F)cn2)C(=O)NCc2ccc(OCC(F)(F)F)cc2)CC1]

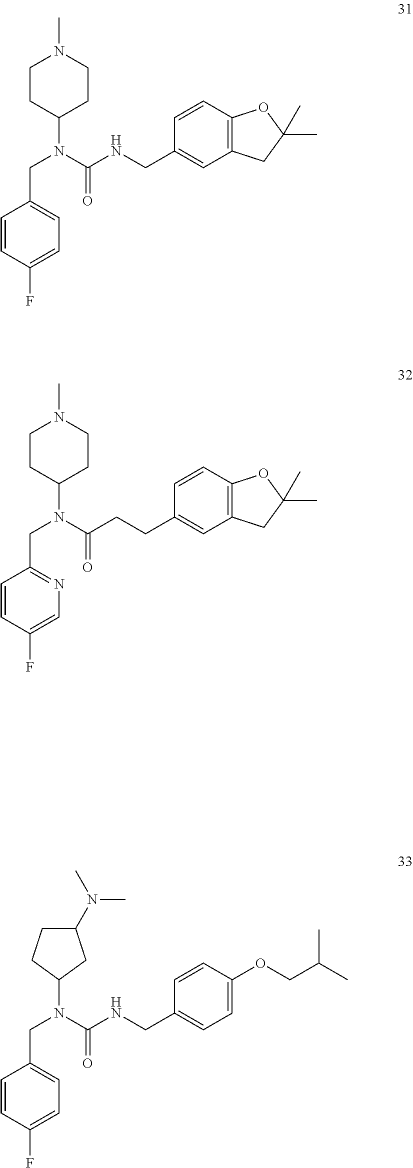 [SMILES: CN1CCC(N(Cc2ccc(F)cc2)C(=O)NCc2ccc3c(c2)CC(C)(C)O3)CC1 ; CN1CCC(N(Cc2ccc(F)cn2)C(=O)CCc2ccc3c(c2)CC(C)(C)O3)CC1 ; CC(C)COc1ccc(CNC(=O)N(Cc2ccc(F)cc2)C2CCC(N(C)C)C2)cc1]

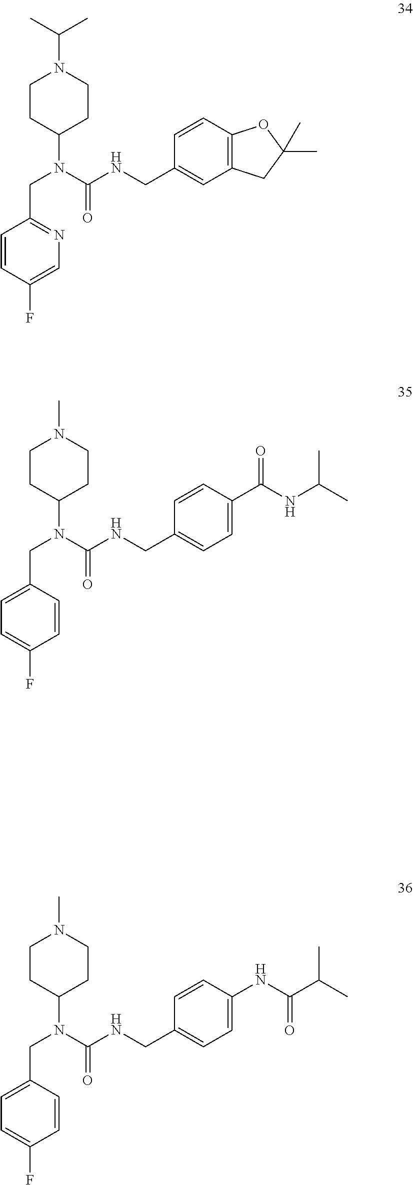 [SMILES: CC(C)N1CCC(N(Cc2ccc(F)cn2)C(=O)NCc2ccc3c(c2)CC(C)(C)O3)CC1 ; CC(C)NC(=O)c1ccc(CNC(=O)N(Cc2ccc(F)cc2)C2CCN(C)CC2)cc1 ; CC(C)C(=O)Nc1ccc(CNC(=O)N(Cc2ccc(F)cc2)C2CCN(C)CC2)cc1]

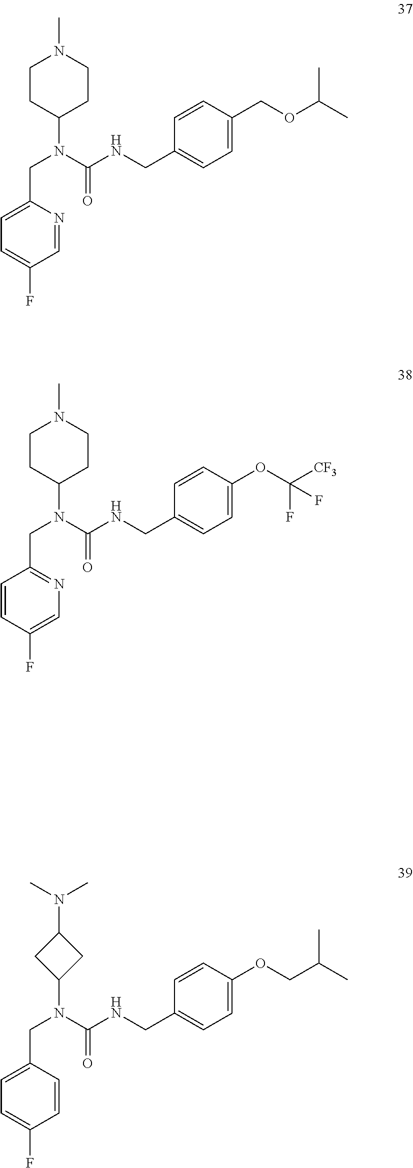 [SMILES: CC(C)OCc1ccc(CNC(=O)N(Cc2ccc(F)cn2)C2CCN(C)CC2)cc1 ; CN1CCC(N(Cc2ccc(F)cn2)C(=O)NCc2ccc(OC(F)(F)C(F)(F)F)cc2)CC1 ; CC(C)COc1ccc(CNC(=O)N(Cc2ccc(F)cc2)C2CC(N(C)C)C2)cc1]

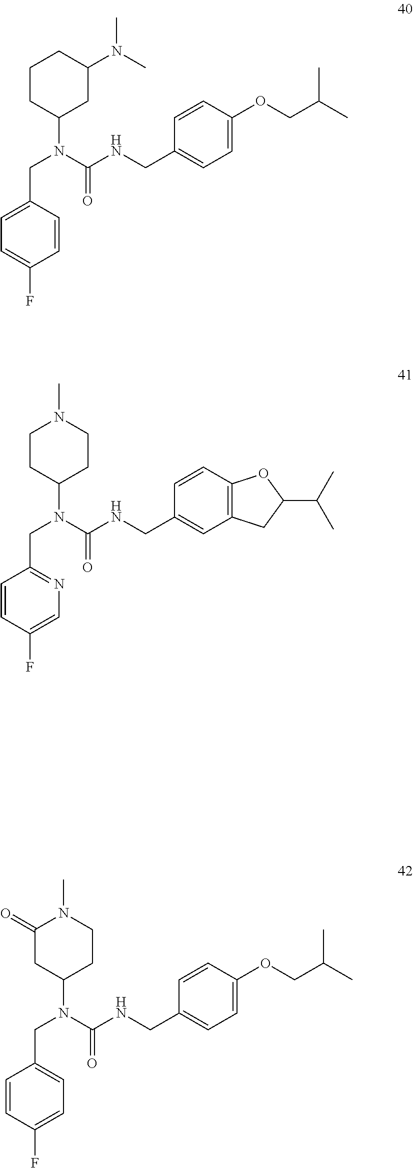 [SMILES: CC(C)COc1ccc(CNC(=O)N(Cc2ccc(F)cc2)C2CCCC(N(C)C)C2)cc1 ; CC(C)C1Cc2cc(CNC(=O)N(Cc3ccc(F)cn3)C3CCN(C)CC3)ccc2O1 ; CC(C)COc1ccc(CNC(=O)N(Cc2ccc(F)cc2)C2CCN(C)C(=O)C2)cc1]

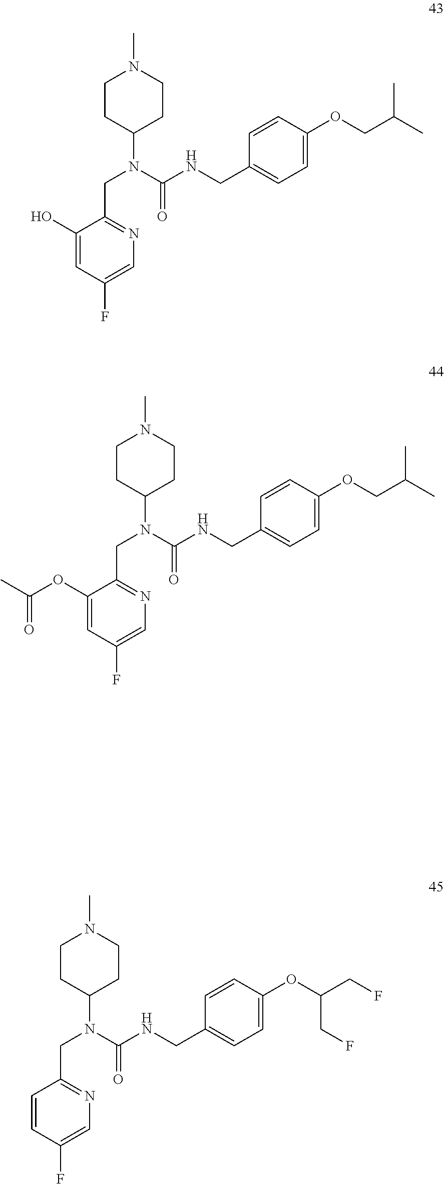

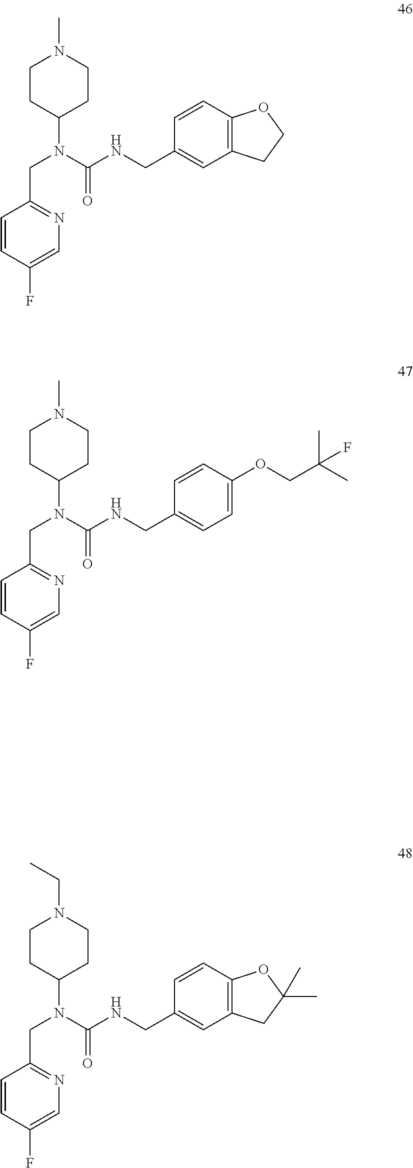 [SMILES: CN1CCC(N(Cc2ccc(F)cn2)C(=O)NCc2ccc3c(c2)CCO3)CC1 ; CN1CCC(N(Cc2ccc(F)cn2)C(=O)NCc2ccc(OCC(C)(C)F)cc2)CC1 ; CCN1CCC(N(Cc2ccc(F)cn2)C(=O)NCc2ccc3c(c2)CC(C)(C)O3)CC1]

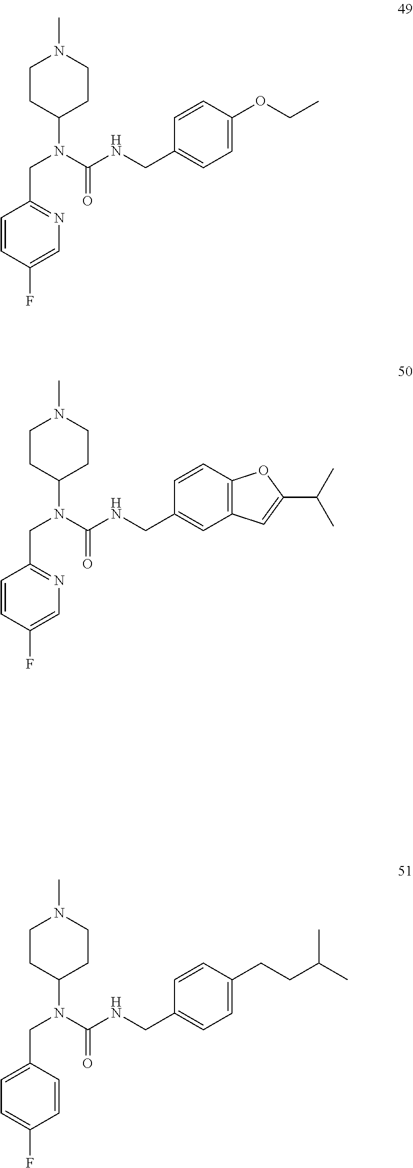 [SMILES: CCOc1ccc(CNC(=O)N(Cc2ccc(F)cn2)C2CCN(C)CC2)cc1 ; CC(C)c1cc2cc(CNC(=O)N(Cc3ccc(F)cn3)C3CCN(C)CC3)ccc2o1 ; CC(C)CCc1ccc(CNC(=O)N(Cc2ccc(F)cc2)C2CCN(C)CC2)cc1]

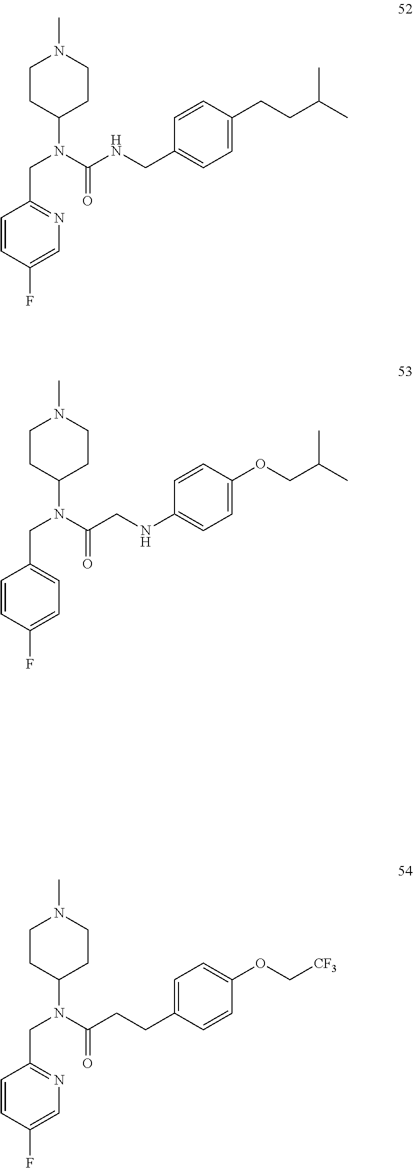 [SMILES: CC(C)CCc1ccc(CNC(=O)N(Cc2ccc(F)cn2)C2CCN(C)CC2)cc1 ; CC(C)COc1ccc(NCC(=O)N(Cc2ccc(F)cc2)C2CCN(C)CC2)cc1 ; CN1CCC(N(Cc2ccc(F)cn2)C(=O)CCc2ccc(OCC(F)(F)F)cc2)CC1]

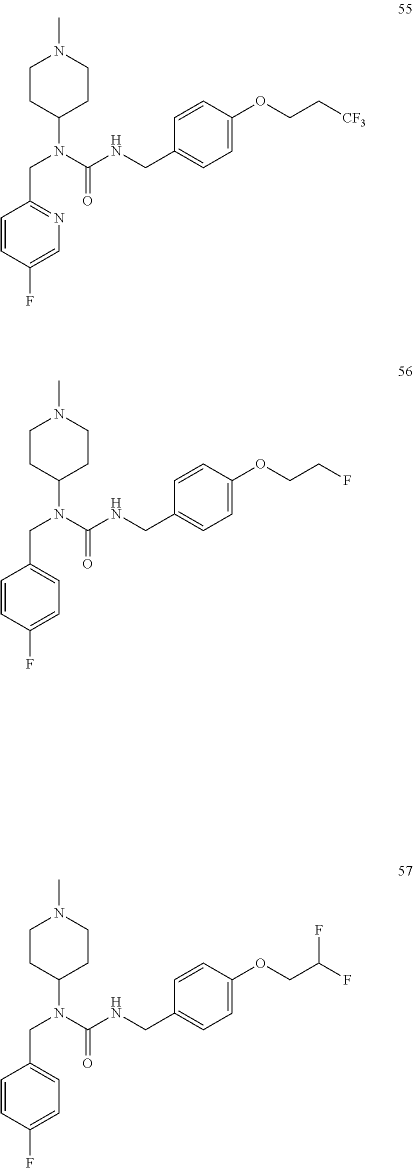 [SMILES: CN1CCC(N(Cc2ccc(F)cn2)C(=O)NCc2ccc(OCCC(F)(F)F)cc2)CC1 ; CN1CCC(N(Cc2ccc(F)cc2)C(=O)NCc2ccc(OCCF)cc2)CC1 ; CN1CCC(N(Cc2ccc(F)cc2)C(=O)NCc2ccc(OCC(F)F)cc2)CC1]

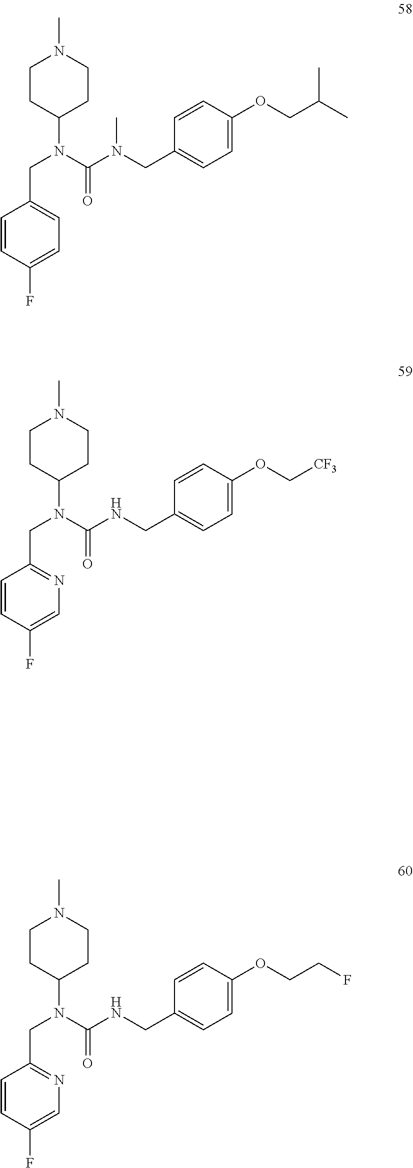 [SMILES: CC(C)COc1ccc(CN(C)C(=O)N(Cc2ccc(F)cc2)C2CCN(C)CC2)cc1 ; CN1CCC(N(Cc2ccc(F)cn2)C(=O)NCc2ccc(OCC(F)(F)F)cc2)CC1 ; CN1CCC(N(Cc2ccc(F)cn2)C(=O)NCc2ccc(OCCF)cc2)CC1]

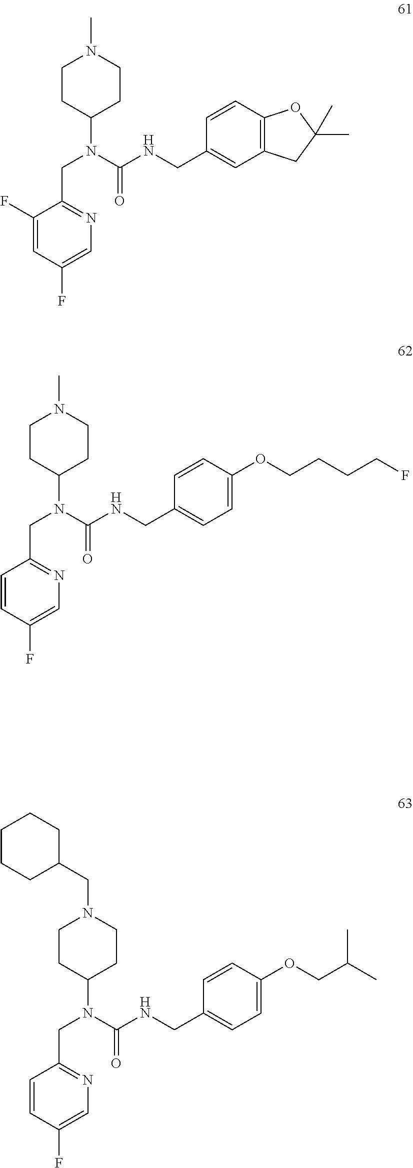 [SMILES: CN1CCC(N(Cc2ncc(F)cc2F)C(=O)NCc2ccc3c(c2)CC(C)(C)O3)CC1 ; CN1CCC(N(Cc2ccc(F)cn2)C(=O)NCc2ccc(OCCCCF)cc2)CC1 ; CC(C)COc1ccc(CNC(=O)N(Cc2ccc(F)cn2)C2CCN(CC3CCCCC3)CC2)cc1]

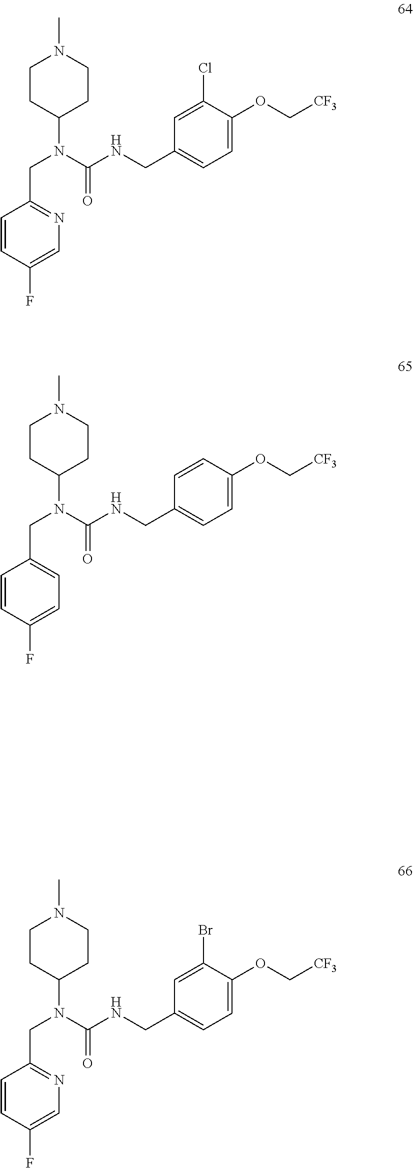 [SMILES: CN1CCC(N(Cc2ccc(F)cn2)C(=O)NCc2ccc(OCC(F)(F)F)c(Cl)c2)CC1 ; CN1CCC(N(Cc2ccc(F)cc2)C(=O)NCc2ccc(OCC(F)(F)F)cc2)CC1 ; CN1CCC(N(Cc2ccc(F)cn2)C(=O)NCc2ccc(OCC(F)(F)F)c(Br)c2)CC1]

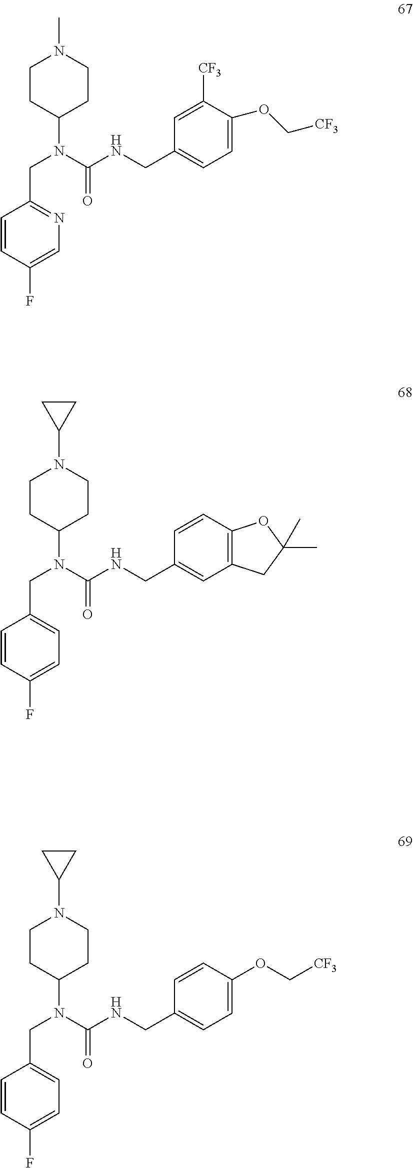 [SMILES: CN1CCC(N(Cc2ccc(F)cn2)C(=O)NCc2ccc(OCC(F)(F)F)c(C(F)(F)F)c2)CC1 ; CC1(C)Cc2cc(CNC(=O)N(Cc3ccc(F)cc3)C3CCN(C4CC4)CC3)ccc2O1 ; O=C(NCc1ccc(OCC(F)(F)F)cc1)N(Cc1ccc(F)cc1)C1CCN(C2CC2)CC1]

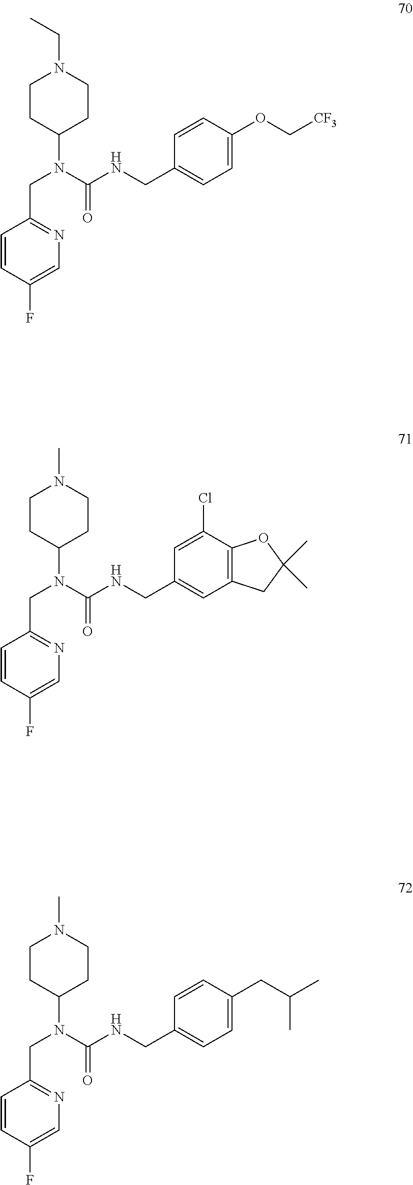 [SMILES: CCN1CCC(N(Cc2ccc(F)cn2)C(=O)NCc2ccc(OCC(F)(F)F)cc2)CC1 ; CN1CCC(N(Cc2ccc(F)cn2)C(=O)NCc2cc(Cl)c3c(c2)CC(C)(C)O3)CC1 ; CC(C)Cc1ccc(CNC(=O)N(Cc2ccc(F)cn2)C2CCN(C)CC2)cc1]

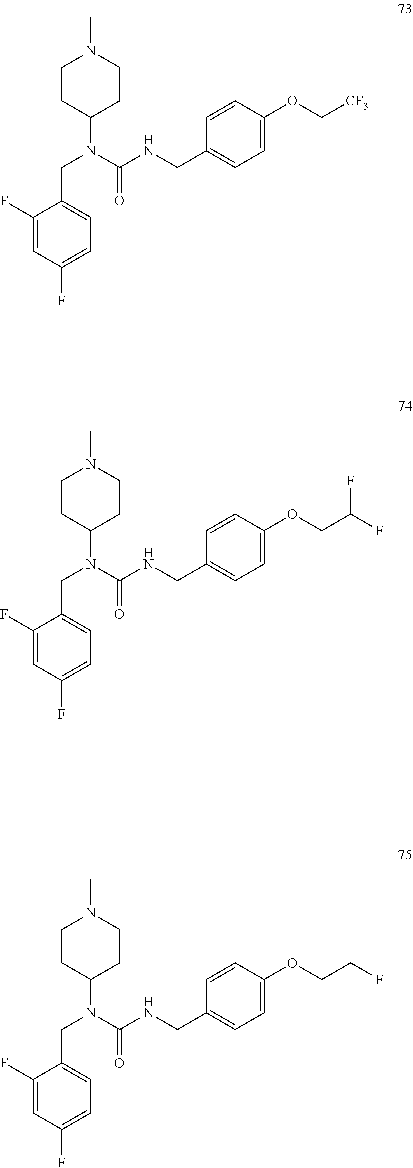 [SMILES: CN1CCC(N(Cc2ccc(F)cc2F)C(=O)NCc2ccc(OCC(F)(F)F)cc2)CC1 ; CN1CCC(N(Cc2ccc(F)cc2F)C(=O)NCc2ccc(OCC(F)F)cc2)CC1 ; CN1CCC(N(Cc2ccc(F)cc2F)C(=O)NCc2ccc(OCCF)cc2)CC1]

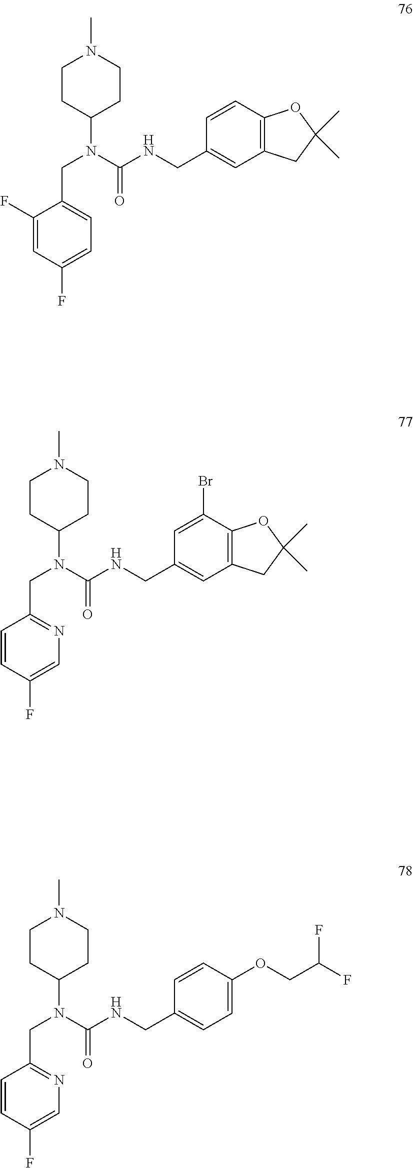 [SMILES: CN1CCC(N(Cc2ccc(F)cc2F)C(=O)NCc2ccc3c(c2)CC(C)(C)O3)CC1 ; CN1CCC(N(Cc2ccc(F)cn2)C(=O)NCc2cc(Br)c3c(c2)CC(C)(C)O3)CC1 ; CN1CCC(N(Cc2ccc(F)cn2)C(=O)NCc2ccc(OCC(F)F)cc2)CC1]

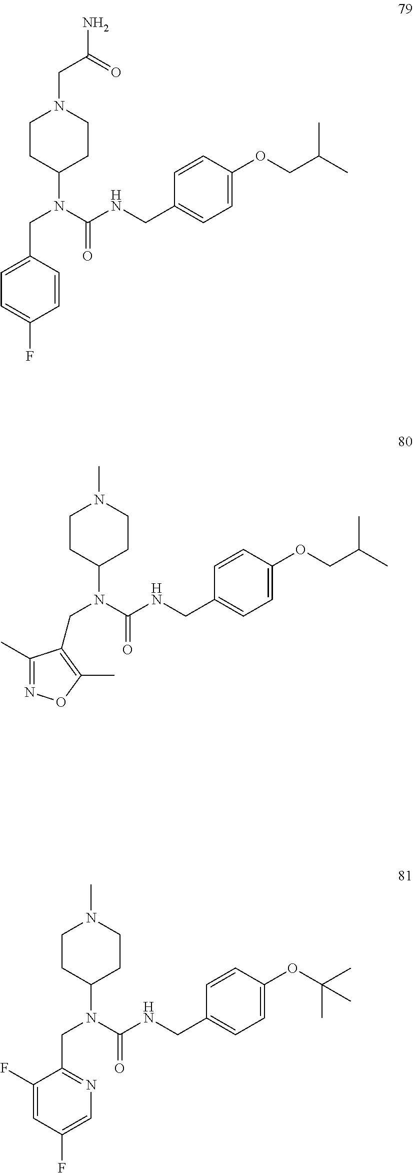

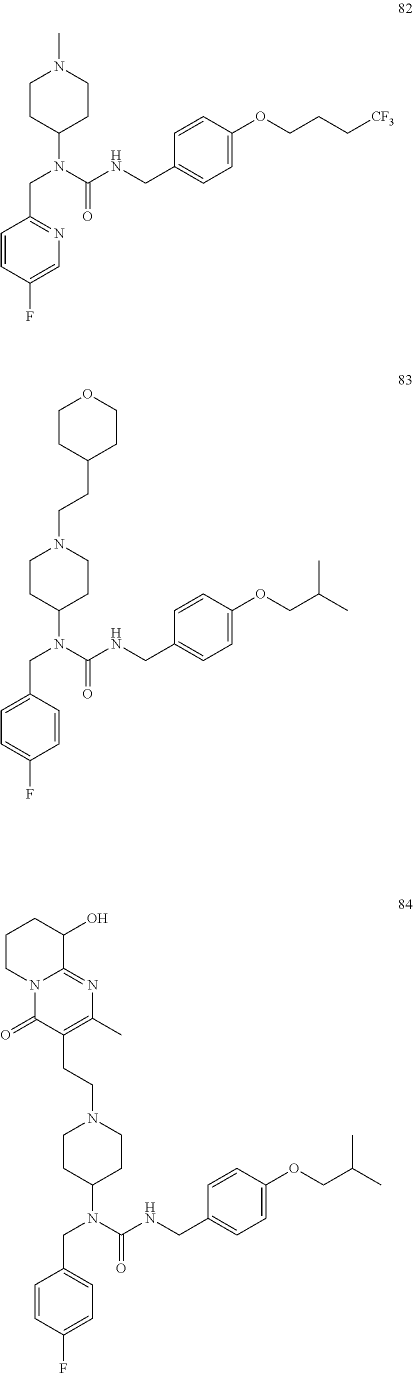 [SMILES: CN1CCC(N(Cc2ccc(F)cn2)C(=O)NCc2ccc(OCCCC(F)(F)F)cc2)CC1 ; CC(C)COc1ccc(CNC(=O)N(Cc2ccc(F)cc2)C2CCN(CCC3CCOCC3)CC2)cc1 ; Cc1nc2n(c(=O)c1CCN1CCC(N(Cc3ccc(F)cc3)C(=O)NCc3ccc(OCC(C)C)cc3)CC1)CCCC2O]

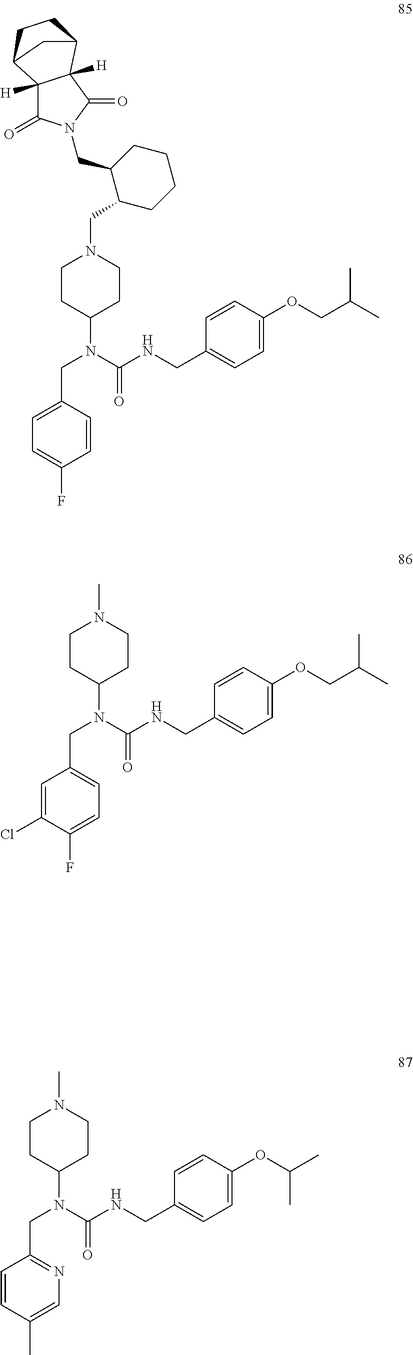 [SMILES: CC(C)COc1ccc(CNC(=O)N(Cc2ccc(F)cc2)C2CCN(C[C@H]3CCCC[C@@H]3CN3C(=O)[C@@H]4[C@H]5CC[C@H](C5)[C@@H]4C3=O)CC2)cc1 ; CC(C)COc1ccc(CNC(=O)N(Cc2ccc(F)c(Cl)c2)C2CCN(C)CC2)cc1 ; Cc1ccc(CN(C(=O)NCc2ccc(OC(C)C)cc2)C2CCN(C)CC2)nc1]

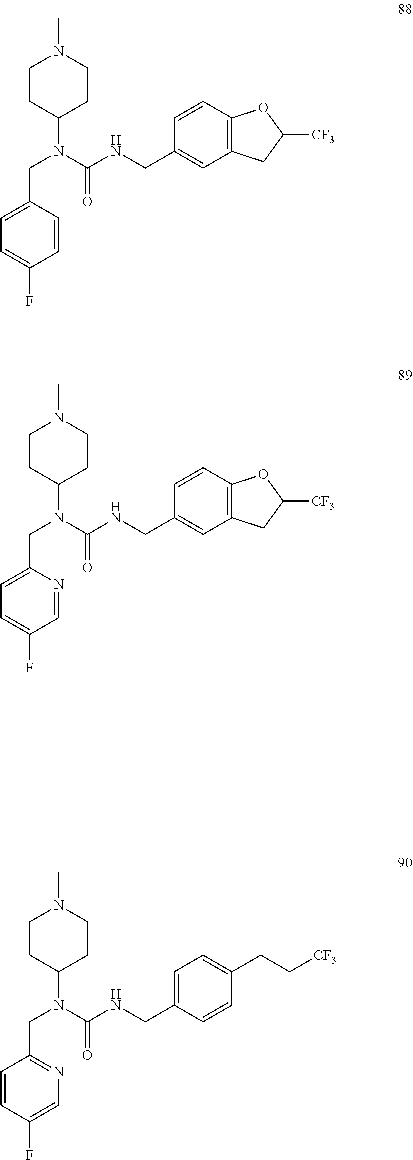 [SMILES: CN1CCC(N(Cc2ccc(F)cc2)C(=O)NCc2ccc3c(c2)CC(C(F)(F)F)O3)CC1 ; CN1CCC(N(Cc2ccc(F)cn2)C(=O)NCc2ccc3c(c2)CC(C(F)(F)F)O3)CC1 ; CN1CCC(N(Cc2ccc(F)cn2)C(=O)NCc2ccc(CCC(F)(F)F)cc2)CC1]

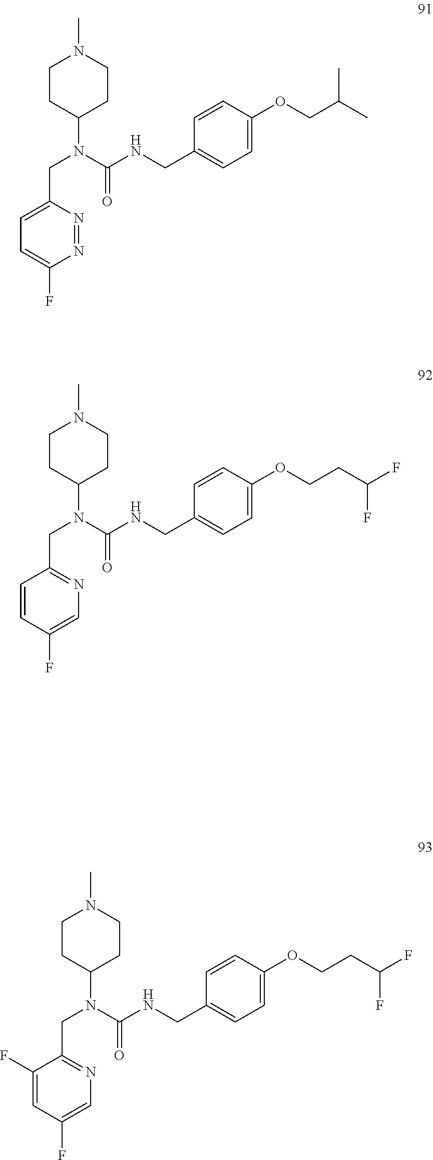 [SMILES: CC(C)COc1ccc(CNC(=O)N(Cc2ccc(F)nn2)C2CCN(C)CC2)cc1 ; CN1CCC(N(Cc2ccc(F)cn2)C(=O)NCc2ccc(OCCC(F)F)cc2)CC1 ; CN1CCC(N(Cc2ncc(F)cc2F)C(=O)NCc2ccc(OCCC(F)F)cc2)CC1]

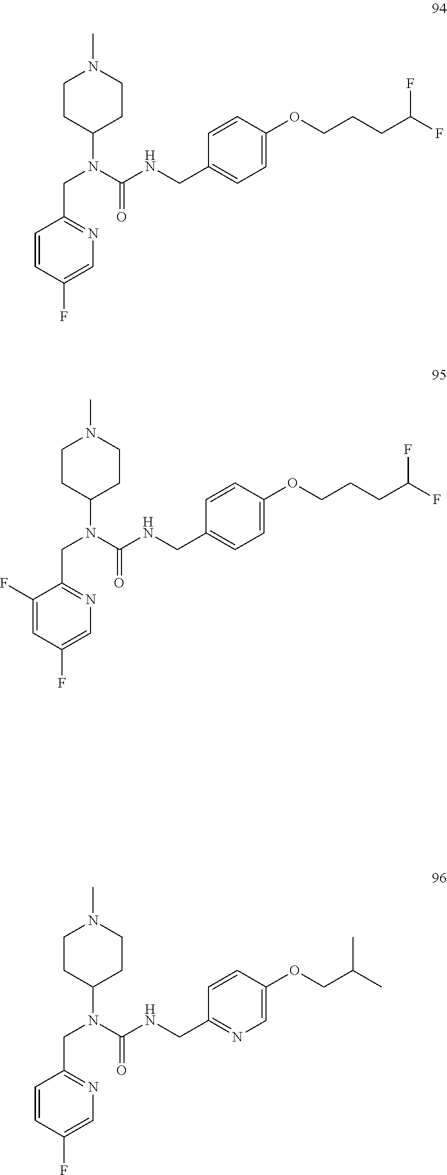 [SMILES: CN1CCC(N(Cc2ccc(F)cn2)C(=O)NCc2ccc(OCCCC(F)F)cc2)CC1 ; CN1CCC(N(Cc2ncc(F)cc2F)C(=O)NCc2ccc(OCCCC(F)F)cc2)CC1 ; CC(C)COc1ccc(CNC(=O)N(Cc2ccc(F)cn2)C2CCN(C)CC2)nc1]

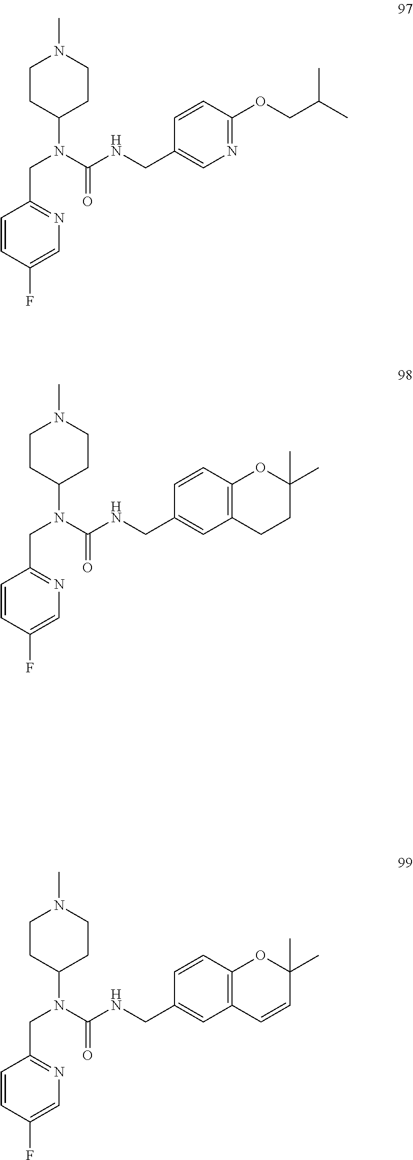 [SMILES: CC(C)COc1ccc(CNC(=O)N(Cc2ccc(F)cn2)C2CCN(C)CC2)cn1 ; CN1CCC(N(Cc2ccc(F)cn2)C(=O)NCc2ccc3c(c2)CCC(C)(C)O3)CC1 ; CN1CCC(N(Cc2ccc(F)cn2)C(=O)NCc2ccc3c(c2)C=CC(C)(C)O3)CC1]

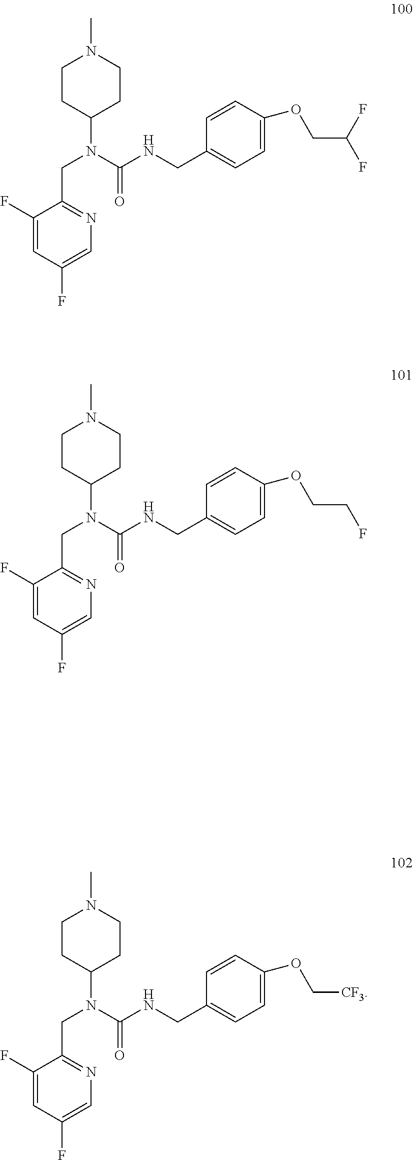 [SMILES: CN1CCC(N(Cc2ncc(F)cc2F)C(=O)NCc2ccc(OCC(F)F)cc2)CC1 ; CN1CCC(N(Cc2ncc(F)cc2F)C(=O)NCc2ccc(OCCF)cc2)CC1 ; CN1CCC(N(Cc2ncc(F)cc2F)C(=O)NCc2ccc(OCC(F)(F)F)cc2)CC1]

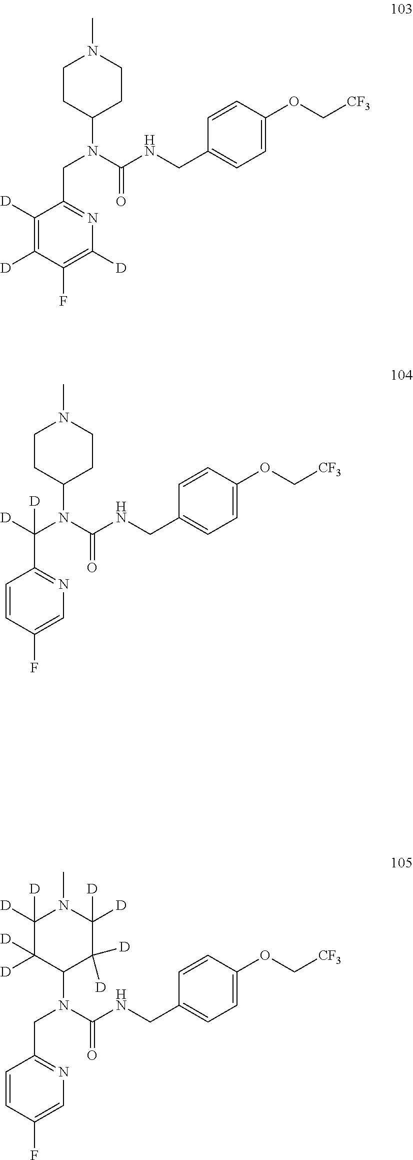 [SMILES: [2H]c1nc(CN(C(=O)NCc2ccc(OCC(F)(F)F)cc2)C2CCN(C)CC2)c([2H])c([2H])c1F ; [2H]C([2H])(c1ccc(F)cn1)N(C(=O)NCc1ccc(OCC(F)(F)F)cc1)C1CCN(C)CC1 ; [2H]C1([2H])C(N(Cc2ccc(F)cn2)C(=O)NCc2ccc(OCC(F)(F)F)cc2)C([2H])([2H])C([2H])([2H])N(C)C1([2H])[2H]]

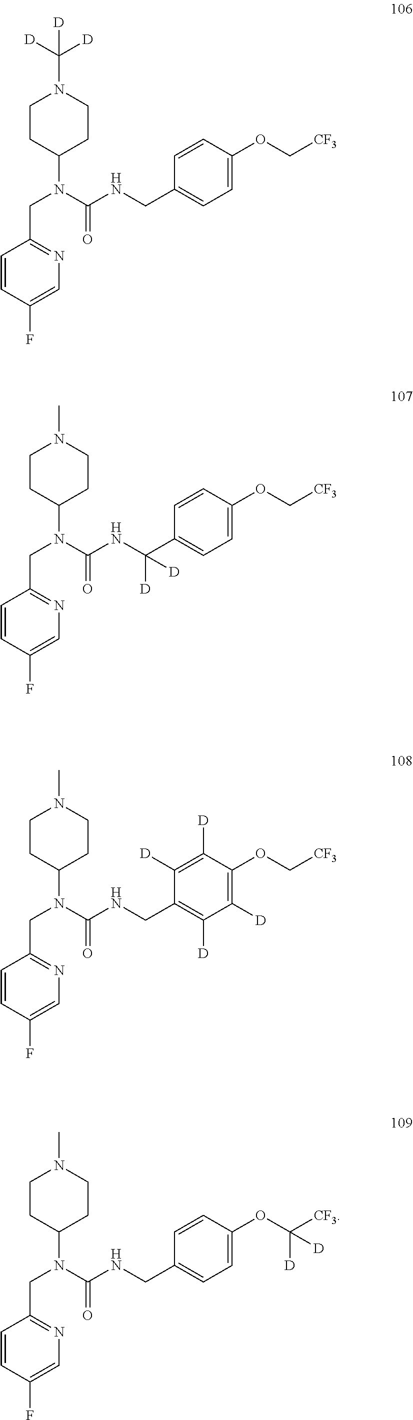 [SMILES: [2H]C([2H])([2H])N1CCC(N(Cc2ccc(F)cn2)C(=O)NCc2ccc(OCC(F)(F)F)cc2)CC1 ; [2H]C([2H])(NC(=O)N(Cc1ccc(F)cn1)C1CCN(C)CC1)c1ccc(OCC(F)(F)F)cc1 ; [2H]c1c([2H])c(OCC(F)(F)F)c([2H])c([2H])c1CNC(=O)N(Cc1ccc(F)cn1)C1CCN(C)CC1 ; [2H]C([2H])(Oc1ccc(CNC(=O)N(Cc2ccc(F)cn2)C2CCN(C)CC2)cc1)C(F)(F)F]

In one aspect, the present invention provides a pharmaceutical composition comprising a therapeutically effective amount of any one of the above-mentioned compounds or the stereoisomers thereof or the pharmaceutically acceptable salts thereof, or a crystalline form of any one of the above-mentioned compounds and a pharmaceutically acceptable carrier. The carrier comprises conventional auxiliary ingredients in the art, such as a filler, a binder, a diluent, a disintegrant, a lubricant, a colorant, a flavoring agent, an antioxidant and a wetting agent.

The pharmaceutical composition can be prepared into various pharmaceutically acceptable dosage forms, such as a tablet, a capsule, an oral liquid, a suspension, a granule, a powder, a microparticle, a pill, a micro-tablet, an instantly soluble film, a nasal spray, a transdermal patch, an injection or various sustained and controlled release preparations. The pharmaceutical composition can be administered orally, transmucosally, rectally or parenterally (including intravascular, intravenous, intraperitoneal, subcutaneous, intramuscular and intrasternal administration). The administration dosage can be appropriately adjusted according to the age, gender and disease type of a patient.

For oral administration, the pharmaceutical composition may be in the form of, for example, a tablet, a capsule, a liquid capsule, a suspension or a liquid. The pharmaceutical composition is preferably prepared in a dosage unit form containing a specified amount of active ingredients. For example, the pharmaceutical composition can be provided as a tablet or a capsule containing active ingredients in an amount ranging from about 0.1 mg to 1000 mg, preferably from about 0.25 mg to 250 mg, and more preferably from about 0.5 mg to 100 mg. The suitable daily dosage for humans or other mammals can vary widely depending on the patient's condition and other factors, but can be determined by means of conventional methods.

In one aspect, the present invention provides the use of any one of the above-mentioned compounds or pharmaceutically acceptable salts thereof or stereoisomers thereof in the preparation of a drug for treating 5-HT_{2A} receptor-related diseases. The diseases or symptoms comprise: schizophrenia, psychosis, schizoaffective disorder, mania, psychotic depression, affective disorder, dementia, anxiety disorder, sleep disorder, dysorexia, bipolar disorder, psychosis secondary to hypertension, migraine, hypertension, thrombosis, vasospasm, ischemia, motor tics, depression, major depressive disorder, anxiety, sleep disturbance, eating disorder, non-motor symptoms caused by Parkinson's disease, delusion, illusion, cognitive disorder, dementia-related mental diseases, negative symptoms of schizophrenia, Parkinson's disease, Huntington's disease, Alzheimer's disease, spinocerebellar ataxia, Tourette's syndrome, Friedreich's ataxia, Machado-Joseph disease, Lewy body dementia, dyskinesia, dysmyotonia, myoclonus, tremor, progressive supranuclear paralysis and frontotemporal dementia, or other disease states and conditions that would have been obvious to a person skilled in the art.

### Definition and Description

Unless otherwise stated, the following terms and phrases used herein are intended to have the following meanings. A specific term or phrase should not be considered uncertain or unclear unless specifically defined, but should be understood in its ordinary meaning. When a trade name appears herein, it is intended to refer to the corresponding commodity or an active ingredient thereof.

The term “pharmaceutically acceptable” as used herein refers to those compounds, materials, compositions and/or dosage forms, which are, within the scope of sound medical judgment, suitable for use in contact with human and animal tissues, without excessive toxicity, irritation, allergic reactions or other problems or complications, which is commensurate with a reasonable benefit/risk ratio.

The term “pharmaceutically acceptable salt” refers to a salt of the compound of the present invention, which is prepared from the compound having specific substituents found in the present invention with relatively non-toxic acids or bases. When compounds of the present invention contain relatively acidic functional groups, base addition salts can be obtained by contacting the neutral form of such compounds with a sufficient amount of base, either in a pure solution or a suitable inert solvent. Examples of pharmaceutically acceptable acid addition salts include inorganic acid salts and organic acid salts, and further include salts of amino acids (e.g., arginine) and salts of organic acids (e.g., glucuronic acid) (see Berge et al., “Pharmaceutical Salts”, Journal of Pharmaceutical Science 66: 1-19 (1977)). Certain specific compounds of the present invention contain basic and acidic functional groups and thus can be converted to any base or acid addition salt.

The pharmaceutically acceptable salts of the present invention can be synthesized from a parent compound containing acid radicals or base radicals by conventional chemical methods. In general, a method for preparing such salts comprises: in water or an organic solvent or a mixture of both, reacting these compounds in free acid or base forms with a stoichiometric amount of a suitable base or acid to prepare the salts.

Certain compounds of the present invention may have asymmetric carbon atoms (optical centers) or double bonds. Racemates, diastereomers, geometric isomers and individual isomers are encompassed within the scope of the present invention.

The compounds of the present invention may exist in specific geometric or stereoisomeric forms. The present invention contemplates all such compounds, including cis and trans isomers, (−)- and (+)-enantiomers, (R)- and (S)-enantiomers, diastereomers, (D)-isomers, (L)-isomers, and racemic mixtures and other mixtures thereof, such as enantiomerically or diastereomerically enriched mixtures, all of which fall within the scope of the present invention. Additional asymmetric carbon atoms may be present in a substituent such as an alkyl group. All these isomers and mixtures thereof are encompassed within the scope of the present invention.

Optically active (R)- and (S)-isomers and D and L isomers can be prepared using chiral synthesis or chiral reagents or other conventional techniques. If a particular enantiomer of a compound of the present invention is desired, it can be prepared by asymmetric synthesis or derivatization with a chiral auxiliary, wherein the resulting diastereomeric mixture is separated and the auxiliary groups are cleaved to provide pure desired enantiomers. Alternatively, where the molecule contains a basic functional group (such as an amino group) or an acidic functional group (such as a carboxyl group), diastereomeric salts can be formed with an appropriate optically active acid or base, followed by resolution of the diastereomers using conventional methods well known in the art, and subsequent recovery of the pure enantiomers. In addition, separation of enantiomers and diastereomers is frequently accomplished by using chromatography, which uses chiral stationary phases, optionally in combination with chemical derivatization methods (e.g., formation of carbamates from amines).

The term “pharmaceutically acceptable carrier” refers to any preparation or carrier medium that can deliver an effective amount of active substances in the present invention, does not interfere with the biological activity of the active substances, and has no toxic or side effects on a host or patient. Representative carriers include, but are not limited to: a binder, a filler, a lubricant, a disintegrant, a wetting agent, a dispersing agent, a solubilizer, a suspending agent, etc.

For a drug or a pharmacologically active agent, the term “effective amount” or “therapeutically effective amount” refers to a sufficient amount of a drug or an agent that is nontoxic but can achieve desired effects. For the oral dosage form in the present invention, the “effective amount” of an active substance in a composition refers to an amount required to achieve desired effects when such active substance is used in combination with another active substance in the composition. Determination of the effective amount, varying from person to person, depends on the age and general condition of a recipient and also depends on a specific active substance. The appropriate effective amount in individual cases can be determined by a person skilled in the art according to conventional tests.

The present invention is intended to include all isotopes of atoms present in the compounds of the present invention. The isotopes comprise those atoms with the same atomic number but different mass numbers. By way of general example, and not as a limitation, isotopes of hydrogen comprise deuterium and tritium. Isotopes of carbon comprise ¹³C and ¹⁴C. Isotope-labeled compounds of the present invention can generally be prepared by using an appropriate isotope-labeled reagent in place of a non-labeled reagent otherwise used by means of conventional techniques known to a person skilled in the art or by means of methods analogous to those described herein.

The term “deuterated analog” refers to an analog formed by substituting one or more hydrogen atoms of a compound with deuterium atoms. The term “optional” or “optionally” means that the subsequently described event or circumstance may, but not necessarily occur, and that the description includes instances where said event or circumstance occurs and instances where said event or circumstance does not occur. For example, “optionally substituted with one or more deuterium atoms” means that the group may be unsubstituted with deuterium atoms or substituted with one or more deuterium atoms, i.e., including the situation that the group is not deuterated, partially deuterated and/or fully deuterated.

The term “substituted” means that any one or more hydrogen atoms on the designated atom are substituted with a substituent, which may include heavy hydrogen and hydrogen variants, provided that the valence state of the designated atom is normal, and the substituted compound is stable. When the substituent is ketone (i.e., ═O), it means that two hydrogen atoms are substituted. Ketone substitution does not occur on aromatic groups.

Where any variable (e.g., R) appears more than once in the composition or structure of a compound, its definition in each case is independent. Thus, for example, if a group is substituted with 0-2 R, the group can optionally be substituted with up to two R, and R in each case has independent options. In addition, combinations of substituents and/or variants thereof are permissible only if such combinations result in stable compounds.

Unless otherwise specified, the term “alkyl” is used to represent a linear or branched saturated hydrocarbon group, which may be monosubstituted (e.g., —CH₂F) or polysubstituted (e.g., —CF₃) and may be monovalent (e.g., methyl), divalent (e.g., methylene) or polyvalent (e.g., methine). For example, C₁-C₁₀ represents 1 to 10 carbons, and C₁₋₁₀ is selected from C₁, C₂, C₃, C₄, C₅, C₆, C₇, C₈, C₉ and C₁₀. Examples of alkyl include methyl (Me), ethyl (Et), propyl (e.g., n-propyl and isopropyl), butyl (e.g., n-butyl, isobutyl, s-butyl and t-butyl), pentyl (e.g., n-pentyl, isopentyl, neopentyl and 1-ethylpropyl), hexyl (e.g., n-hexyl, isohexyl, 1,1-dimethylbutyl, 2,2-dimethylbutyl, 3,3-dimethylbutyl and 2-ethylbutyl), heptyl, octyl, nonyl, decyl, etc.

Unless otherwise specified, the term “halo” or “halogen” by itself or as part of another substituent means a fluorine, chlorine, bromine or iodine atom. The term “haloalkoxy” is intended to include monohaloalkoxy and linear or branched polyhaloalkoxy. For example, the term “C₁₋₁₀ haloalkoxy” is intended to include, but is not limited to, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy and 6,6,6-trifluorohexyloxy.

The term “haloalkyl” is intended to include monohaloalkyl and linear or branched polyhaloalkyl. For example, the term “(C₁-C₄) haloalkyl” is intended to include, but is not limited to, trifluoromethyl, 2,2,2-trifluoroethyl, 4-chlorobutyl, 3-bromopropyl, etc. Unless otherwise specified, examples of haloalkyl include, but are not limited to: trifluoromethyl, trichloromethyl, pentafluoroethyl and pentachloroethyl.

Unless otherwise specified, the “alkoxy” represents the above alkyl having a specific number of carbon atoms connected via an oxygen bridge. Typical alkoxy includes C₁₋₁₀ alkoxy, such as C₁ alkoxy, C₂ alkoxy, C₃ alkoxy, C₄ alkoxy, C₅ alkoxy, C₆ alkoxy, C₇ alkoxy, C₈ alkoxy, C₉ alkoxy and C₁₀ alkoxy. Examples of alkoxy include, but are not limited to: methoxy, ethoxy, n-propoxy, isopropoxy, n-butoxy, sec-butoxy, tert-butoxy, n-pentoxy, S-pentoxy, hexyloxy, 2-ethylbutoxy, heptyloxy, octyloxy, nonyloxy, decyloxy, etc.

Unless otherwise specified, cycloalkyl includes any stable cyclic or polycyclic hydrocarbon group, and any carbon atom is saturated, which may be monosubstituted or polysubstituted and may be monovalent, divalent or polyvalent. Examples of the cycloalkyl include, but are not limited to, cyclopropyl, cyclobutyl, cyclopentyl, cyclohexyl, cycloheptyl, cyclooctyl, norbornyl, [2.2.2] bicyclooctane, [4.4.0] bicyclodecane, etc.

Compounds are named by hand or ChemDraw® software, and commercially available compounds are named by the supplier catalog names.

### BRIEF DESCRIPTION OF THE DRAWINGS

FIG. 1: Comparison diagram of drug-time curves of pimavanserin and compound 59 after intragastric administration

### DETAILED DESCRIPTION OF EMBODIMENTS

The present invention is further described below in conjunction with specific examples and test examples, but the scope of the present invention is not limited in any way.

### Example 1

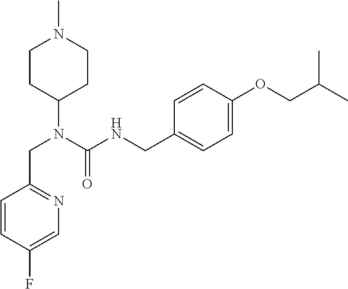 [SMILES: CC(C)COc1ccc(CNC(=O)N(Cc2ccc(F)cn2)C2CCN(C)CC2)cc1]

### Synthesis Route:

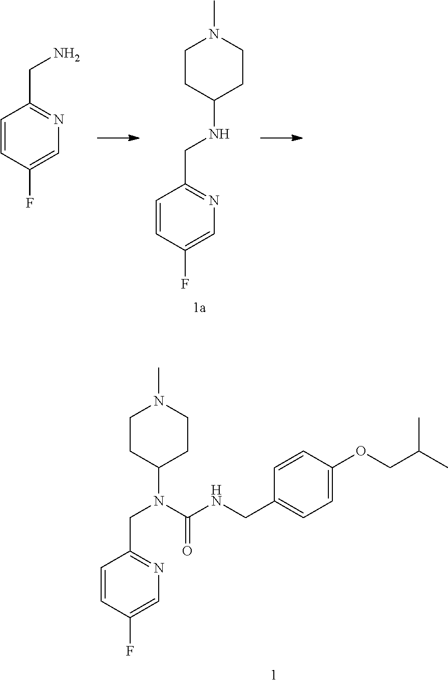 [SMILES: NCc1ccc(F)cn1 ; CN1CCC(NCc2ccc(F)cn2)CC1 ; CC(C)COc1ccc(CNC(=O)N(Cc2ccc(F)cn2)C2CCN(C)CC2)cc1] [0170] 1. Under nitrogen protection, in an ice-water bath, 2-(aminomethyl)-5-fluoropyridine (504 mg, 4.0 mmol) was dissolved in 10 ml of methanol. N-methyl-4-piperidone (452 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 1a (538 mg), which was directly used in the next reaction without purification.
2. Under nitrogen protection, compound 1a (446 mg, 2.0 mmol) was dissolved in 10 ml of acetonitrile. N-(4-isobutyloxybenzyl)-1H-imidazol-formamide (546 mg, 2.0 mmol) and potassium carbonate (414 mg, 3.0 mmol) were added, and the resulting mixture was heated to 60° C. and reacted under stirring for 12 h. The reaction solution was cooled to room temperature and filtered. 20 ml of water was added to the filtrate, and then the resulting solution was extracted with dichloromethane (10 mL*3). The organic phases were combined, dried over anhydrous sodium sulfate, filtered, concentrated under reduced pressure and separated by column chromatography (dichloromethane:methanol=10:1) to obtain compound 1 (414 mg, a light yellow solid, yield: 480). MS m/z (ESI): 429.3 [M+1]; ¹H NMR (400 MHz, CDCl₃) δ 8.26 (d, 1H), 7.38-7.32 (m, 1H), 7.31-7.28 (m, 1H), 7.18-7.14 (m, 2H), 6.85-6.80 (m, 2H), 6.64-6.58 (m, 1H), 4.37 (s, 2H), 4.34 (d, 2H), 3.70 (d, 2H), 2.93-2.87 (m, 2H), 2.28 (s, 3H), 2.13-1.98 (m, 4H), 1.81-1.64 (m, 4H), 1.02 (d, 6H).

Compounds 2-3, 12-13, 16, 19-20, 23-31, 33-52, 58, 61, 64, 66-68, 70-72, 75-77, 80-81 and 83-101 were prepared in a similar manner to example 1.

TABLE 1
Structures and characterization data of compounds 2-3, 12-13, 16, 19-20, 23-31,
33-52, 58, 61, 64, 66-68, 70-72, 75-77, 80-81 and 83-101

| Compound | | |
|---|---|---|
| number | Structural formula | Characterization data |
| 2 | 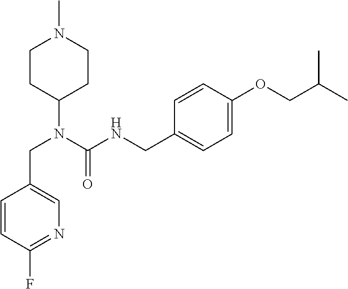 [SMILES: CC(C)COc1ccc(CNC(=O)N(Cc2ccc(F)nc2)C2CCN(C)CC2)cc1] | MS m/z (ESI): 429.3 [M + 1]; ¹H NMR(400 MHz, CDCl₃) δ 8.1(s, 1H), 7.69-7.65(m, 1H), 7.06-7.02(m, 2H), 6.90-6.87(m, 1H), 6.81-6.77(m, 2H), 4.69-4.60(m, 2H), 4.46(s, 2H), 4.29(d, 2H), 3.68(d, 2H), 3.45-3.38(m, 2H), 2.84-2.74(m, 2H), 2.70(s, 3H), 2.52- 2.41(m, 2H), 2.09-2.02(m, 1H), 1.88- 1.81(m, 2H), 1.01(d,6H). |
| 3 | 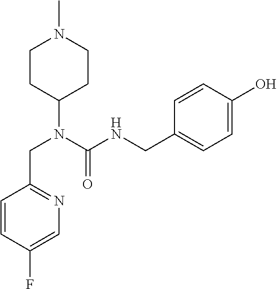 [SMILES: CN1CCC(N(Cc2ccc(F)cn2)C(=O)NCc2ccc(O)cc2)CC1] | MS m/z (ESI): 373.2[M + 1]¹H NMR (600 MHz, DMSO-d₆) δ 9.23 (s, 1H), 8.48 (d, 1H), 7.81-7.48 (m, 1H), 7.33 (m, 1H), 7.03 (m, 3H), 6.67 (d, 2H), 4.46 (s, 2H), 4.25-4.17 (m, 1H), 4.14 (d, 2H), 2.76 (d, 2H), 2.62-2.46 (m, 3H), 1.88 (m, ,2H), 1.68-1.58 (m, 2H), 1.25 (d, 2H). |
| 12 | 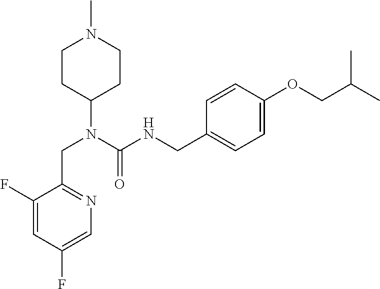 [SMILES: CC(C)COc1ccc(CNC(=O)N(Cc2ncc(F)cc2F)C2CCN(C)CC2)cc1] | MS m/z (ESI): 447.3 [M + 1]; ¹H NMR(400 MHz,CDCl₃) δ 8.15-8.13(m, 1H), 7.23-7.17(m, 3H), 6.96-6.92(m, 1H), 6.85-6.80(m, 2H), 4.46(s, 2H), 4.35-4.32(m, 2H), 4.30-4.22(m, 1H), 3.68(d, 2H), 3.04-2.98(m, 2H), 2.37(s, 3H), 2.26-2.20(m, 2H), 2.09-2.02(m, 1H), 1.96-1.88(m, 2H), 1.70-1.65(m, 2H), 1.01(d, 6H) |
| 13 | 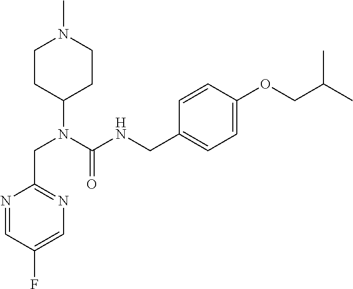 [SMILES: CC(C)COc1ccc(CNC(=O)N(Cc2ncc(F)cn2)C2CCN(C)CC2)cc1] | MS m/z (ESI): 430.3 [M + 1]; ¹H NMR(400 MHz,CDCl₃) δ 8.48(s, 2H), 7.16-7.13(m, 2H), 6.85-6.80(m, 2H), 6.42-6.37(m, 1H), 4.56(s, 2H), 4.52- 4.47(m, 1H), 4.33(d, 2H), 3.70(d, 2H), 3.40-3.36(m, 2H), 2.74-2.71(m, 1H), 2.65(s, 3H), 2.50-2.41(m, 3H), 2.09- 2.02(m, 1H), 1.88-1.81(m, 2H), 1.01(d, 6H). |
| 16 | 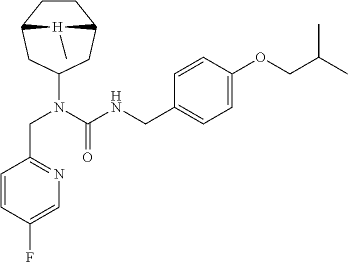 | MS m/z (ESI): 455.3 [M + 1]; ¹H NMR(400 MHz, CDCl₃) δ 8.23(s, 1H), 7.72-7.68(m, 1H), 7.45-7.38(m, 1H), 7.25-7.20(m, 2H), 6.87-6.84(m, 2H), 4.67-4.62(m, 1H), 4.44(s, 2H), 4.35- 4.30(d, 2H) 3.71(d, 2H), 3.63-3.58(m, 1H), 3.47-3.44(m, 1H), 2.74-2.71(m, 1H), 2.64-2.60(m, 2H), 2.50(s, 3H), 2.25-2.17(m, 3H), 2.13-2.05(m, 2H), 1.65-1.61(m, 2H), 1.03(d, 6H). |
| 19 | 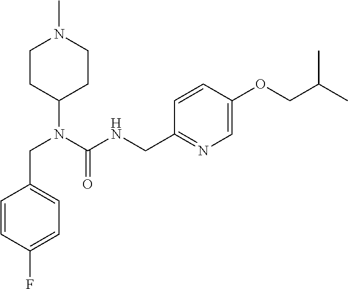 [SMILES: CC(C)COc1ccc(CNC(=O)N(Cc2ccc(F)cc2)C2CCN(C)CC2)nc1] | MS m/z (ESI): 429.3 [M + 1]; ¹H NMR(400 MHz, CDCl₃) δ 8.10(s, 1H), 7.25-7.20(m, 2H), 7.10-7.05(m, 2H), 6.99-6.89(m, 2H), 5.50-5.42(m, 1H), 4.45-4.35(m, 4H), 4.30-4.20(m, 1H), 3.70(d, 2H), 2.88-2.85(m, 2H), 2.26(s, 3H), 2.09-2.00(m, 3H), 1.75-1.60(m, 4H), 1.02(d, 6H). |
| 20 | 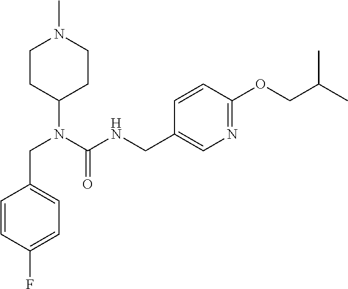 [SMILES: CC(C)COc1ccc(CNC(=O)N(Cc2ccc(F)cc2)C2CCN(C)CC2)cn1] | MS m/z (ESI): 429.3 [M + 1]; ¹H NMR(400 MHz, CDCl₃) δ 7.91(s, 1H), 7.38-7.33(m, 1H), 7.24-7.20(m, 2H), 7.05-6.95(m, 2H), 6.69-6.65(m, 1H), 4.55-4.45(m, 1H), 4.40-4.20(m, 5H), 3.99(d, 2H), 2.98(d, 2H), 2.30(s, 3H), 2.19-2.00(m, 3H), 1.75-1.65(m, 4H), 1.01(d, 6H). |
| 21 | 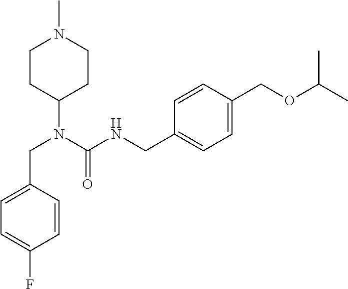 [SMILES: CC(C)OCc1ccc(CNC(=O)N(Cc2ccc(F)cc2)C2CCN(C)CC2)cc1] | MS m/z (ESI): 428.3 [M + 1]; ¹H NMR(400 MHz, CDCl₃) δ 7.25- 7.15(m, 4H), 7.10-6.97(m, 4H), 4.55- 4.45(m, 1H), 4.42(s, 2H), 4.40-4.25(m, 5H), 3.70-3.63(m, 1H), 2.88(d, 2H), 2.28(s, 3H), 2.19-2.09(m, 2H), 1.75- 1.65(m, 4H), 1.01(d, 6H). |
| 23 | 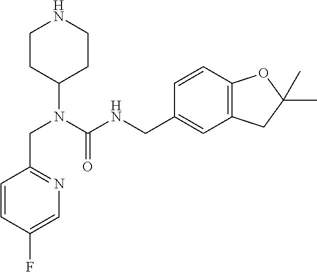 [SMILES: CC1(C)Cc2cc(CNC(=O)N(Cc3ccc(F)cn3)C3CCNCC3)ccc2O1] | MS m/z (ESI): 413.2 [M + 1]; ¹H NMR(400 MHz, CDCl₃) δ 9.49- 9.34(m, 2H), 8.26(s, 1H), 7.41-7.38(m, 2H), 7.07-7.02(m, 2H), 6.68-6.66(m, 1H), 4.58-4.44(m, 1H), 4.41-4.32(m, 4H), 3.45(d, 2H), 3.00-2.88(m, 4H), 2.30-2.24(m, 2H), 1.75-1.68(m, 2H), 1.47(s, 6H). |
| 24 | 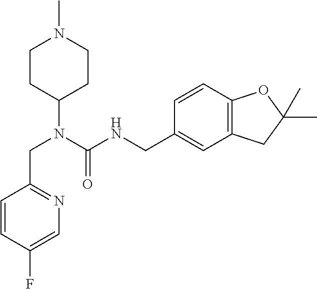 [SMILES: CN1CCC(N(Cc2ccc(F)cn2)C(=O)NCc2ccc3c(c2)CC(C)(C)O3)CC1] | MS m/z (ESI): 427.2 [M + 1]; ¹H NMR(400 MHz, CDCl₃) δ 8.51(s, 1H), 7.62-7.54(m, 2H), 7.24-7.20(m, 1H), 6.89-6.77(m, 2H), 4.63(s, 2H), 4.58- 4.44(m, 3H), 3.25-3.10(m, 4H), 2.80- 2.71(m, 1H), 2.52(s, 3H), 2.30-2.24(m, 2H), 2.08-1.88(m, 4H), 1.70(s, 6H). |
| 25 | 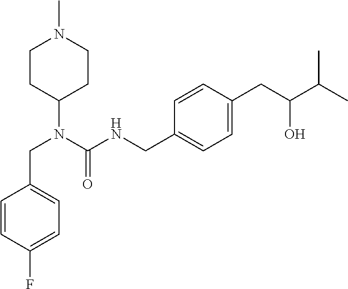 [SMILES: CC(C)C(O)Cc1ccc(CNC(=O)N(Cc2ccc(F)cc2)C2CCN(C)CC2)cc1] | MS m/z (ESI): 442.3 [M + 1]; ¹H NMR(400 MHz, DMSO-d₆) δ 7.25- 7.21(m, 2H), 7.15-7.03(m, 6H), 6.94- 6.82(m, 1H), 4.42(s, 2H), 4.38-4.29(m, 1H), 4.24-4.18(m, 2H), 3.94-3.81(m, 1H), 3.48-3.32(m, 1H), 2.74(d, 2H), 2.70-2.58(m, 2H), 2.10(s, 3H), 1.90- 1.75(m, 2H), 1.59-1.41(m, 5H), 0.87(d, 6H). |
| 26 | 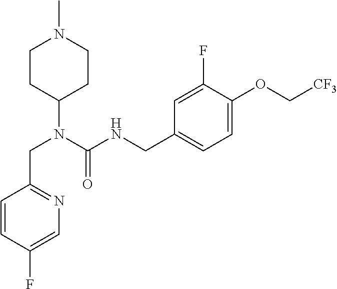 [SMILES: CN1CCC(N(Cc2ccc(F)cn2)C(=O)NCc2ccc(OCC(F)(F)F)c(F)c2)CC1] | MS m/z (ESI): 473.2 [M + 1]; ¹H NMR(400 MHz, CDCl₃) δ 8.28(d, 1H), 7.88(s, 1H), 7.70-7.65(m, 1H), 7.46- 7.42(m, 1H), 7.13-7.08(m, 1H), 7.05- 6.97(m, 2H), 4.60-4.53(m, 1H), 4.50- 4.45(s, 2H), 4.43-4.37(m, 4H), 3.46- 3.40(m, 2H), 2.80-2.69(m, 5H), 2.67- 2.58(m, 2H), 1.79-1.73(m, 2H). |
| 27 | 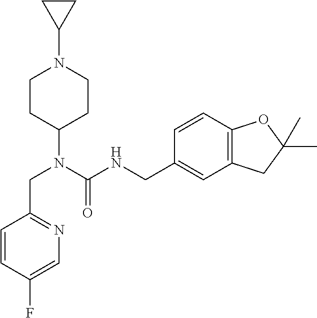 [SMILES: CC1(C)Cc2cc(CNC(=O)N(Cc3ccc(F)cn3)C3CCN(C4CC4)CC3)ccc2O1] | MS m/z (ESI): 453.3 [M + 1]; ¹H NMR(400 MHz, CDCl₃) δ 8.27(s, 1H), 7.42-7.34(m, 2H), 7.05-6.98(m, 2H), 6.67-6.64(m, 1H), 4.43-4.34(m, 5H), 3.25-3.10(m, 2H), 3.00-2.95(m, 2H), 2.50-2.31(m, 2H), 1.82-1.60(m, 5H), 1.48(s, 6H), 0.63-0.35(m, 4H). |
| 28 | 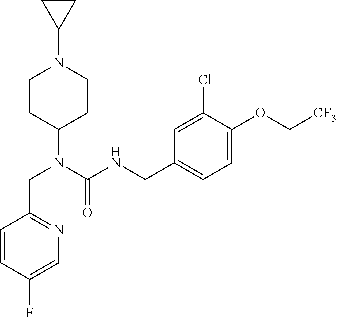 [SMILES: O=C(NCc1ccc(OCC(F)(F)F)c(Cl)c1)N(Cc1ccc(F)cn1)C1CCN(C2CC2)CC1] | MS m/z (ESI): 515.2 [M + 1]; ¹H NMR(400 MHz, CDCl₃) δ 8.33(d, 1H), 7.44-7.29(m, 3H), 7.17-7.13(m, 1H), 6.94-6.90(m, 1H), 4.50-4.44(m, 3H), 4.42-4.34(m, 4H), 3.26-3.10(m, 2H), 2.40-2.30(m, 2H), 1.89-1.63(m, 5H), 0.58-0.38(m, 4H). |
| 29 | 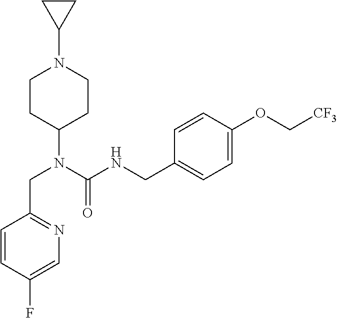 [SMILES: O=C(NCc1ccc(OCC(F)(F)F)cc1)N(Cc1ccc(F)cn1)C1CCN(C2CC2)CC1] | MS m/z (ESI): 480.2 [M + 1]; ¹H NMR(400 MHz, CDCl₃) δ 8.38(d, 1H), 7.45-7.28(m, 4H), 7.22-7.16(m, 1H), 6.99-6.90(m, 1H), 4.43-4.24(m, 4H), 3.96-3.90(m, 2H), 3.17-3.08(m, 2H), 2.40-2.30(m, 2H), 1.95-1.85(m, 1H), 1.79-1.60(m, 5H), 0.52-0.40(m, 4H). |
| 30 | 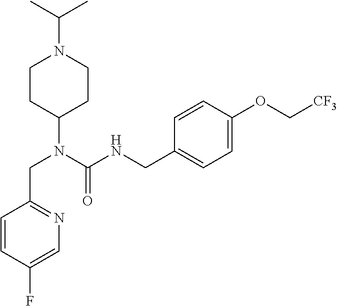 | MS m/z (ESI): 473.2 [M + 1]; ¹H NMR(400 MHz, CDCl₃) δ 8.23(d, 1H), 7.98(s, 1H), 7.80-7.75(m, 1H), 7.43- 7.40(m, 1H), 7.32-7.27(m, 2H), 6.95- 6.90(m, 2H), 4.61-4.55(m, 1H), 4.53- 4.49(s, 2H), 4.41-4.32(m, 4H), 3.44- 3.38(m, 3H), 2.90-2.78(m, 4H), 1.79- 1.73(m, 2H), 1.42(d, 6H). |
| 31 | 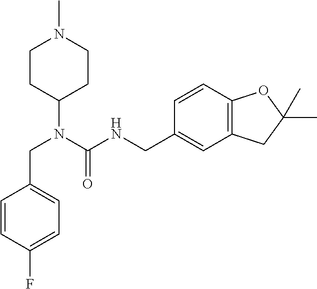 [SMILES: CN1CCC(N(Cc2ccc(F)cc2)C(=O)NCc2ccc3c(c2)CC(C)(C)O3)CC1] | MS m/z (ESI): 426.2 [M + 1]; ¹H NMR(400 MHz, CDCl₃) δ 7.22- 7.17(m, 2H), 7.02-6.98(m, 2H), 6.84- 6.81(m, 2H), 6.62-6.58(m, 1H), 4.55- 4.47(m, 1H), 4.37(s, 2H), 4.26-4.24(m, 2H), 3.15-3.08(m, 2H), 2.96-2.91(m, 2H), 2.45(s, 3H), 2.40-2.34(m, 2H), 2.04-1.94(m, 2H), 1.82-1.76(m, 2H), 1.46(s, 6H). |
| 33 | 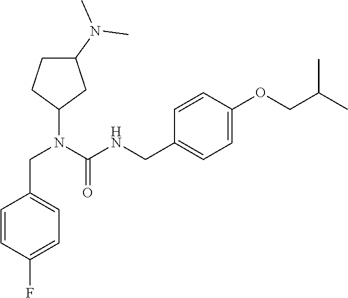 [SMILES: CC(C)COc1ccc(CNC(=O)N(Cc2ccc(F)cc2)C2CCC(N(C)C)C2)cc1] | MS m/z (ESI): 442.3 [M + 1]; ¹H NMR(400 MHz, DMSO-d₆) δ 7.28- 7.22(m, 2H), 7.19-7.08(m, 4H), 6.88- 6.76(m, 2H), 4.58-4.41(m, 4H), 4.24- 4.20(m, 2H), 3.71(d, 2H), 2.35-2.20(m, 2H), 2.17(s, 6H), 2.06-1.92(m, 3H), 1.70-1.52(m, 2H), 1.44-1.38(m, 1H), 0.97(d, 6H). |
| 34 | 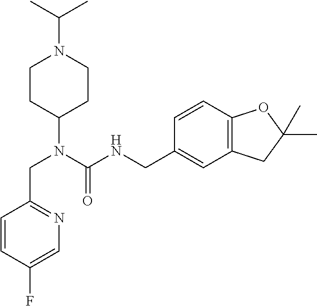 [SMILES: CC(C)N1CCC(N(Cc2ccc(F)cn2)C(=O)NCc2ccc3c(c2)CC(C)(C)O3)CC1] | MS m/z (ESI): 455.3 [M + 1]; ¹H NMR(400 MHz, DMSO-d₆) δ 8.47(s, 1H), 7.72-7.64(m, 1H), 7.35-7.28(m, 1H), 7.07-6.94(m, 3H), 6.68-6.65(m, 1H), 4.50(s, 1H), 4.23-4.04(m, 3H), 2.95(s, 2H), 2.80-2.60(m, 3H), 2.30- 2.11(m, 2H), 1.62-1.45(m, 4H), 1.38(s, 6H), 0.97(d, 6H). |
| 35 | 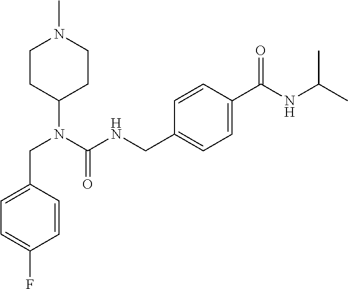 [SMILES: CC(C)NC(=O)c1ccc(CNC(=O)N(Cc2ccc(F)cc2)C2CCN(C)CC2)cc1] | MS m/z (ESI): 441.3 [M + 1]; ¹H NMR(400 MHz, DMSO-d₆) δ 8.21- 8.12(m, 1H), 7.82-7.76(m, 2H), 7.26- 7.20(m, 4H), 7.12-7.01(m, 3H), 4.45- 4.29(m, 4H), 4.18-3.94(m, 2H), 2.78- 2.68(m, 2H), 2.11(s, 3H), 1.87-1.65(m, 2H), 1.60-1.42(m, 4H), 0.99(d, 6H). |
| 36 | 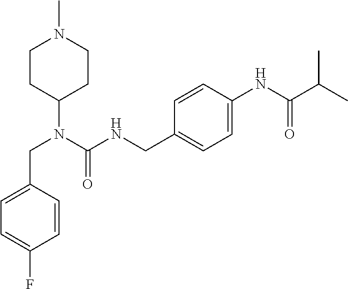 [SMILES: CC(C)C(=O)Nc1ccc(CNC(=O)N(Cc2ccc(F)cc2)C2CCN(C)CC2)cc1] | MS m/z (ESI): 441.3 [M + 1]; ¹H NMR(400 MHz, DMSO-d₆) δ 9.76(s, 1H), 7.52-7.46(m, 2H), 7.26-7.22(m, 2H), 7.12-7.01(m, 4H), 6.93-6.88(m, 1H), 4.45-4.39(m, 2H), 4.18-4.14(m, 2H), 3.99-3.90(m, 1H), 2.78-2.73(m, 2H), 2.60-2.52(m, 1H), 2.15(s, 3H), 2.01-1.88(m, 2H), 1.61-1.42(m, 4H), 1.01 (d, 6H). |
| 37 | 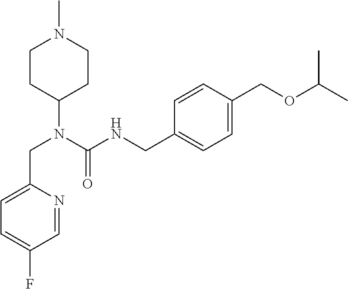 [SMILES: CC(C)OCc1ccc(CNC(=O)N(Cc2ccc(F)cn2)C2CCN(C)CC2)cc1] | MS m/z (ESI): 429.3 [M + 1]; ¹H NMR(400 MHz, DMSO-d₆) δ 8.46(d, 1H), 7.68-7.62(m, 1H), 7.31-7.28(m, 1H), 7.22-7.10(m, 6H), 4.50-4.40(m, 4H), 4.24(d, 2H), 4.00-3.87(m, 1H), 3.70-3.60(m, 1H), 2.83-2.77(m, 2H), 2.21(s, 3H), 2.03-1.88(m, 2H), 1.61- 1.44(m, 4H), 1.12(d, 6H). |
| 38 | 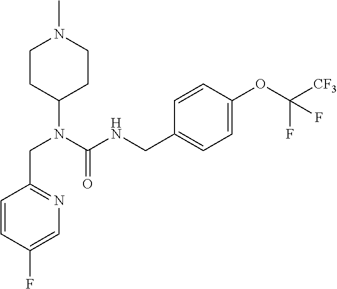 [SMILES: CN1CCC(N(Cc2ccc(F)cn2)C(=O)NCc2ccc(OC(F)(F)C(F)(F)F)cc2)CC1] | MS m/z (ESI): 491.2 [M + 1]; ¹H NMR(400 MHz, DMSO-d₆) δ 8.49(d, 1H), 7.66-7.61(m, 1H), 7.36-7.18(m, 6H), 4.50(s, 2H), 4.36-4.30(m, 2H), 4.00-3.87(m, 1H), 2.81-2.72(m, 2H), 2.11(s, 3H), 2.03-1.88(m, 2H), 1.63- 1.45(m, 4H). |
| 39 | 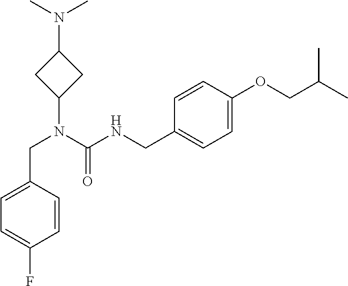 [SMILES: CC(C)COc1ccc(CNC(=O)N(Cc2ccc(F)cc2)C2CC(N(C)C)C2)cc1] | MS m/z (ESI): 428.3 [M + 1]; ¹H NMR(400 MHz, CDCl₃) δ 7.25- 7.02(m, 7H), 6.88-6.76(m, 2H), 4.58- 4.41(m, 4H), 4.26-4.21(m, 2H), 3.65(d, 2H), 2.45-2.30(m, 3H), 2.13(s, 6H), 2.06-1.99(m, 1H), 1.44-1.38(m, 1H), 0.97(d, 6H). |
| 40 | 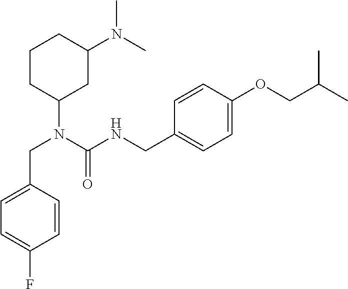 [SMILES: CC(C)COc1ccc(CNC(=O)N(Cc2ccc(F)cc2)C2CCCC(N(C)C)C2)cc1] | MS m/z (ESI): 456.3 [M + 1]; ¹H NMR(400 MHz, DMSO-d₆) δ 7.27- 7.23(m, 2H), 7.20-7.06(m, 4H), 6.85- 6.76(m, 2H), 4.44-4.25(m, 3H), 4.20- 4.07(m, 2H), 3.73(d, 2H), 3.30-3.21(m, 1H), 2.17(s, 6H), 2.06-1.97(m, 1H), 1.80-1.52(m, 4H), 1.47-1.31(m, 3H), 0.98(d, 6H). |
| 41 | 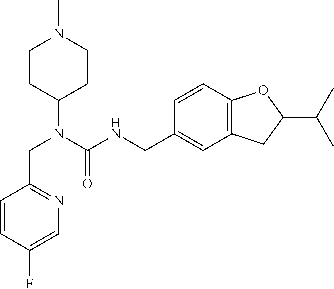 [SMILES: CC(C)C1Cc2cc(CNC(=O)N(Cc3ccc(F)cn3)C3CCN(C)CC3)ccc2O1] | MS m/z (ESI): 441.3 [M + 1]; ¹H NMR(400 MHz, CDCl₃) δ 8.23(d, 1H), 7.90-7.60(s, 1H), 7.50-7.32(m, 3H), 7.08-6.86(m, 2H), 6.65-6.60(m, 1H), 4.50-4.31(m, 4H), 4.26-4.21(m, 2H), 3.25(d, 2H), 3.12-2.92(m, 2H), 2.69- 2.55(m, 4H), 2.46-2.29(m, 2H), 1.84- 1.68(m, 3H), 0.95(d, 6H). |
| 42 | 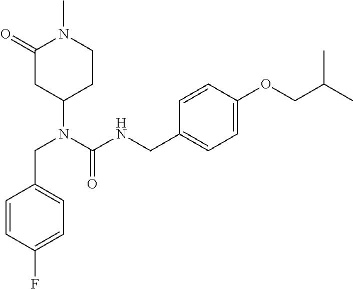 [SMILES: CC(C)COc1ccc(CNC(=O)N(Cc2ccc(F)cc2)C2CCN(C)C(=O)C2)cc1] | MS m/z (ESI): 442.2 [M + 1]; ¹H NMR(400 MHz, DMSO-d₆) δ 7.28- 7.12(m, 6H), 7.01-6.95(m, 1H), 6.88- 6.79(m, 2H), 4.48-4.31(m, 3H), 4.26- 4.21(m, 2H), 3.72(d, 2H), 3.25-3.15(m, 2H), 2.75(s, 3H), 2.35-2.20(m, 2H), 2.01-1.79(m, 2H), 1.64-1.58(m, 1H), 0.95(d, 6H). |
| 43 | 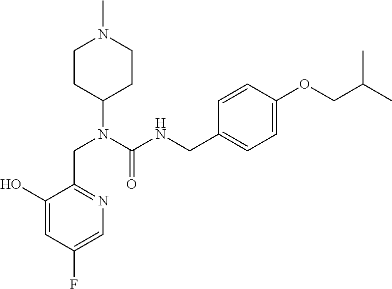 [SMILES: CC(C)COc1ccc(CNC(=O)N(Cc2ncc(F)cc2O)C2CCN(C)CC2)cc1] | MS m/z (ESI): 445.2 [M + 1]; ¹H NMR(400 MHz, CDCl₃) δ 7.98(d, 1H), 7.48-7.36(m, 3H), 7.01-6.95(m, 3H), 4.68-4.54(m, 4H), 4.01-3.85(m, 3H), 3.22(d, 2H), 2.55(s, 3H), 2.39-2.20(m, 5H), 1.79-1.73(m, 2H), 1.15(d, 6H). |
| 44 | 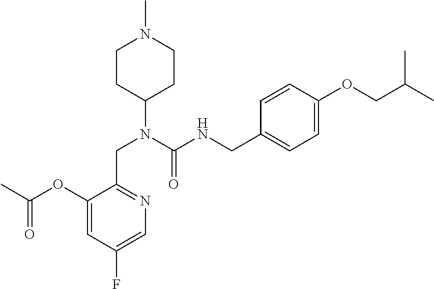 [SMILES: CC(=O)Oc1cc(F)cnc1CN(C(=O)NCc1ccc(OCC(C)C)cc1)C1CCN(C)CC1] | MS m/z (ESI): 487.3 [M + 1]; ¹H NMR(400 MHz, CDCl₃) δ 8.22(d, 1H), 7.38-7.32(m, 1H), 7.21-7.05(m, 3H), 6.80-6.73(m, 2H), 4.48-4.34(m, 5H), 3.71(d, 2H), 3.52(d, 2H), 2.95-2.75(m, 5H), 2.45(s, 3H), 2.39-2.33(m, 2H), 2.12-2.00(m, 1H), 1.79-1.73(m, 2H), 1.00(d, 6H). |
| 45 | 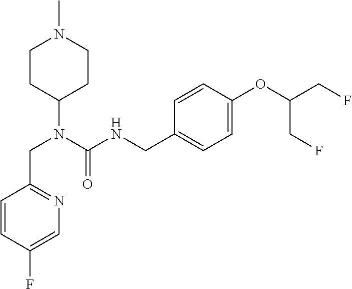 [SMILES: CN1CCC(N(Cc2ccc(F)cn2)C(=O)NCc2ccc(OC(CF)CF)cc2)CC1] | MS m/z (ESI): 451.2 [M + 1]; ¹H NMR(400 MHz, DMSO-d₆) δ 8.48(d, 1H), 7.70-7.65(m, 1H), 7.32-7.27(m, 1H), 7.18-7.07(m, 3H), 6.95-6.88(m, 2H), 4.94-4.60(m, 5H), 4.44(s, 2H), 4.23(d, 2H), 3.96-3.86(m, 1H), 2.72(d, 2H), 2.12(s, 3H), 1.89-1.75(m, 2H), 1.55-1.43(m, 4H). |
| 46 | 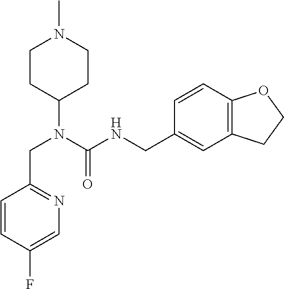 [SMILES: CN1CCC(N(Cc2ccc(F)cn2)C(=O)NCc2ccc3c(c2)CCO3)CC1] | MS m/z (ESI): 399.2 [M + 1]; ¹H NMR(400 MHz, CDCl₃) δ 8.21(d, 1H), 7.35-7.30(m, 2H), 7.10-6.92(m, 3H), 6.65-6.60(m, 1H), 4.52-4.41(m, 2H), 4.36-4.14(m, 5H), 3.15-2.99(m, 4H), 2.42(s, 3H), 2.35-2.20(m, 2H), 2.06- 1.86(m, 2H), 1.64-1.58(m, 2H). |
| 47 | 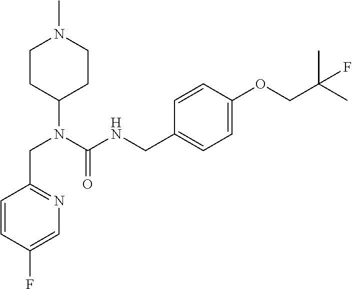 [SMILES: CN1CCC(N(Cc2ccc(F)cn2)C(=O)NCc2ccc(OCC(C)(C)F)cc2)CC1] | MS m/z (ESI): 447.3 [M + 1]; ¹H NMR(400 MHz, MeOD-d₄) δ 8.38(d, 1H), 7.55-7.48(m, 1H), 7.32-7.28(m, 1H), 7.25-7.17(m, 2H), 6.85-6.78(m, 2H), 4.51(s, 2H), 4.27(s, 2H), 4.20- 4.11(m, 1H), 3.92(s, 2H), 2.98(d, 2H), 2.34(s, 3H), 2.28-2.22(m, 2H), 1.79- 1.65(m, 4H), 1.45(d, 6H). |
| 48 | 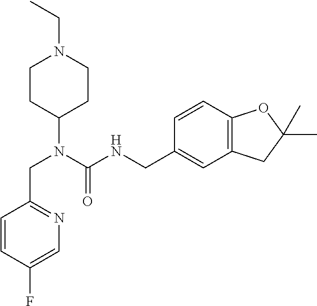 [SMILES: CCN1CCC(N(Cc2ccc(F)cn2)C(=O)NCc2ccc3c(c2)CC(C)(C)O3)CC1] | MS m/z (ESI): 441.3 [M + 1]; ¹H NMR(400 MHz, CDCl₃) δ 8.26(s, 1H), 7.42-7.34(m, 2H), 7.04-6.90(m, 2H), 6.75-6.65(m, 2H), 4.43(s, 2H), 4.35- 4.27(m, 3H), 3.10(d, 2H), 2.95(s, 2H), 2.60-2.51(m, 2H), 2.20-2.11(m, 2H), 1.98-1.78(m, 2H), 1.74-1.67(m, 2H), 1.48(s, 6H), 1.14(t, 3H). |
| 49 | 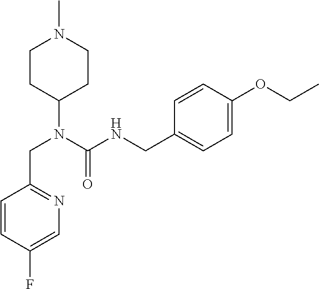 [SMILES: CCOc1ccc(CNC(=O)N(Cc2ccc(F)cn2)C2CCN(C)CC2)cc1] | MS m/z (ESI): 401.2 [M + 1]; ¹H NMR(400 MHz, CDCl₃) δ 8.25(d, 1H), 7.39-7.29(m, 2H), 7.21-7.13(m, 2H), 6.85-6.80(m, 2H), 6.74-6.64(m, 1H), 4.37-4.24(m, 5H), 3.99(q, 2H), 2.93- 2.85(d, 2H), 2.27(s, 3H), 2.13-1.98(m, 2H), 1.81-1.64(m, 4H), 1.42(t, 3H). |
| 50 | 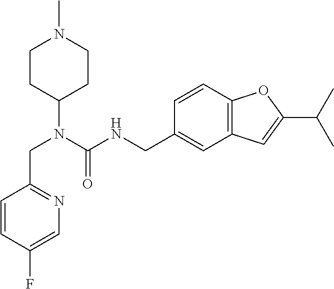 [SMILES: CC(C)c1cc2cc(CNC(=O)N(Cc3ccc(F)cn3)C3CCN(C)CC3)ccc2o1] | MS m/z (ESI): 439.3 [M + 1]; ¹H NMR(400 MHz, CDCl₃) δ 8.25(d, 1H), 7.40-7.32(m, 4H), 7.09-7.06(m, 1H), 6.79-6.70(m, 1H), 6.29(s, 1H), 4.50- 4.44(d, 2H), 4.41-4.25(m, 3H), 3.12- 3.04(m, 1H), 2.93(d, 2H), 2.30(s, 3H), 2.16-2.09(m, 2H), 1.84-1.65(m, 4H), 1.29(d, 6H). |
| 51 | 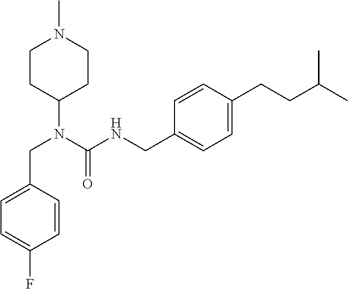 [SMILES: CC(C)CCc1ccc(CNC(=O)N(Cc2ccc(F)cc2)C2CCN(C)CC2)cc1] | MS m/z (ESI): 426.2 [M + 1]; ¹H NMR(400 MHz, CDCl₃) δ 7.22- 7.16(m, 2H), 7.09-6.88(m, 7H), 4.54- 4.47(m, 1H), 4.36-4.24(m, 5H), 2.91(d, 2H), 2.65-2.52(m, 2H), 2.27(s, 3H), 2.20-2.05(m, 2H), 1.80-1.66(m, 4H), 1.60-1.42(m, 3H), 0.91(d, 6H). |
| 52 | 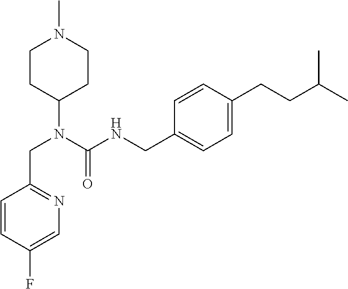 [SMILES: CC(C)CCc1ccc(CNC(=O)N(Cc2ccc(F)cn2)C2CCN(C)CC2)cc1] | MS m/z (ESI): 427.2 [M + 1]; ¹H NMR(400 MHz, CDCl₃) δ 8.24(d, 1H), 7.55-7.50(m, 1H), 7.45-7.35(m, 2H), 7.20-7.10(m, 4H), 4.55-4.47(m, 1H), 4.42-4.34(m, 4H), 3.28(d, 2H), 2.65- 2.52(m, 7H), 2.40-2.31(m, 2H), 1.76- 1.70(m, 2H), 1.60-1.45(m, 3H), 0.93(d, 6H). |
| 58 | 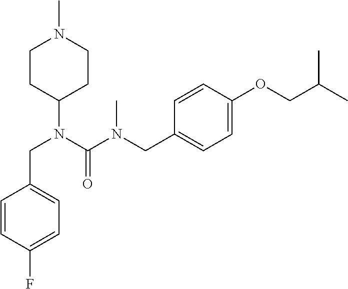 [SMILES: CC(C)COc1ccc(CN(C)C(=O)N(Cc2ccc(F)cc2)C2CCN(C)CC2)cc1] | MS m/z (ESI): 442.3 [M + 1]; ¹H NMR(400 MHz, DMSO-d₆) δ 7.28- 7.24(m, 2H), 7.15-6.97(m, 4H), 6.85- 6.75(m, 2H), 4.25-4.15(m, 4H), 3.70(d, 2H), 2.80-2.70(m, 2H), 2.63(s, 3H), 2.08(s, 3H), 2.05-1.90(m, 2H), 1.88- 1.68(m, 4H), 1.61-1.49(m, 2H), 0.99(d, 6H). |
| 61 | 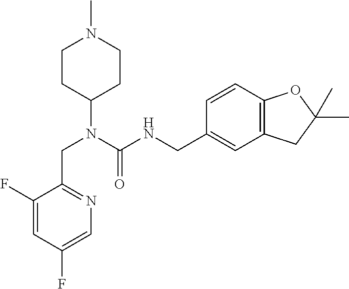 [SMILES: CN1CCC(N(Cc2ncc(F)cc2F)C(=O)NCc2ccc3c(c2)CC(C)(C)O3)CC1] | MS m/z (ESI): 445.2[M + 1], 467.23[M + 23]¹H NMR (600 MHz, CDCl₃) δ 8.13 (d, 1H), 7.23 (m, 1H), 7.04 (d, 1H), 7.01-6.98 (m, 1H), 6.93 (s, 1H), 6.65 (d, 1H), 4.48 (d, 2H), 4.30 (d, 2H), 3.48 (s, 1H), 3.34 (d, 2H), 2.97 (s, 2H), 2.62 (s, 5H), 2.37- 2.30 (m, 2H), 1.82-1.76 (m, 2H), 1.46 (s, 6H). |
| 64 | 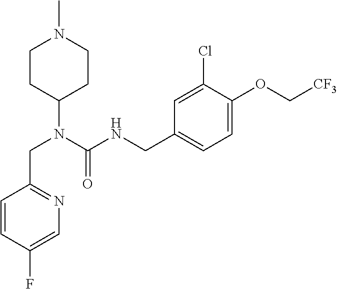 [SMILES: CN1CCC(N(Cc2ccc(F)cn2)C(=O)NCc2ccc(OCC(F)(F)F)c(Cl)c2)CC1] | MS m/z (ESI): 489.2[M + 1]¹H NMR (600 MHz, DMSO-d₆) δ 8.49 (d, 1H), 7.74-7.56 (m, 1H), 7.33 (m, 1H), 7.22- 7.16 (m, 3H), 7.02 (s, 1H), 4.83 (d, 2H), 4.48 (s, 2H), 4.19 (d, 2H), 4.13 (d, 1H), 3.43-3.28 (m, 2H), 3.09-2.96 (m, 2H), 2.41 (s, 3H), 1.83-1.75 (m, 2H), 1.69- 1.48 (m, 2H). |
| 66 | 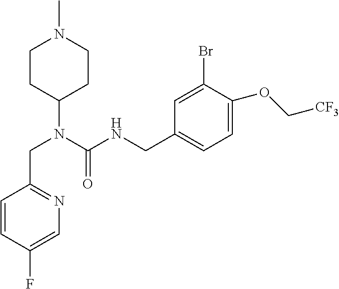 [SMILES: CN1CCC(N(Cc2ccc(F)cn2)C(=O)NCc2ccc(OCC(F)(F)F)c(Br)c2)CC1] | MS m/z (ESI): 534.2[M + 1]¹H NMR (600 MHz, DMSO-d₆) δ 8.48 (m, 1H), 7.71-7.60 (m, 1H), 7.32 (m, 1H), 7.26- 7.11 (m, 3H), 7.02-6.94 (m, 1H), 4.82 (d, 1H), 4.71 (d, 1H), 4.47 (d, 2H), 4.19 (t, 2H), 4.06 (s, 1H), 3.32 (s, 1H), 2.94 (s, 2H), 2.31 (s, 4H), 1.70 (d, 2H), 1.52 (d, 2H). |
| 67 | 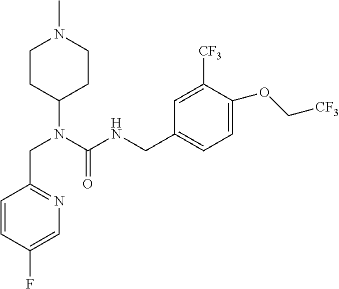 [SMILES: CN1CCC(N(Cc2ccc(F)cn2)C(=O)NCc2ccc(OCC(F)(F)F)c(C(F)(F)F)c2)CC1] | MS m/z (ESI): 523.4[M + 1], 545.2[M + 23] ¹H NMR (600 MHz, DMSO-d₆) δ 8.48 (s, 1H), 7.65 (m, 1H), 7.54-7.47 (m, 1H), 7.35-7.29 (m, 1H), 6.97 (m, 3H), 4.91 (d, 1H), 4.70 (d, 1H), 4.48 (d, 2H), 4.24 (d, 1H), 4.17 (d, 1H), 4.07 (s, 1H), 3.37-3.30 (m, 1H), 3.07 (s, 2H), 2.43 (s, 3H), 2.13 (s, 1H), 1.79 (d, 2H), 1.58 (d, 2H). |
| 68 | 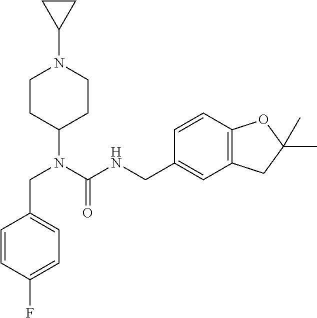 [SMILES: CC1(C)Cc2cc(CNC(=O)N(Cc3ccc(F)cc3)C3CCN(C4CC4)CC3)ccc2O1] | MS m/z (ESI): 452.5 [M + 1] ¹H NMR (600 MHz, DMSO-d₆) δ 7.22 (m, 2H), 7.15-7.01 (m, 2H), 6.98-6.87 (m, 2H), 6.81 (t, 1H), 6.58 (d, 1H), 4.39 (s, 2H), 4.15 (d, 2H), 4.05 (d, 1H), 2.95 (d, 3H), 2.88 (s, 2H), 2.78 (s, 1H), 2.16 (s, 1H), 1.44 (s, 4H), 1.38 (d, 10H). |
| 70 | 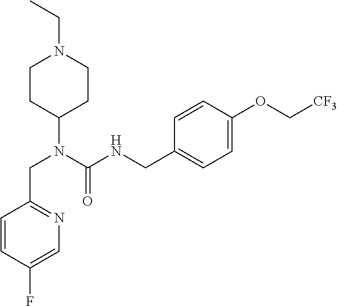 [SMILES: CCN1CCC(N(Cc2ccc(F)cn2)C(=O)NCc2ccc(OCC(F)(F)F)cc2)CC1] | MS m/z (ESI): 469.1[M + 1]¹H NMR (600 MHz, DMSO-d₆) δ 8.49 (d, 1H), 7.69 (d, 1H), 7.34 (d, 1H), 7.23-7.15 (m, 3H), 7.03-6.94 (m, 2H), 4.71 (t, 2H), 4.48 (s, 2H), 4.20 (d, 3H), 3.32 (s, 2H), 2.81(m, 2H), 2.50 (m, 2H), 2.00- 1.81 (m, 2H), 1.62 (s, 2H), 1.18-1.07 (m, 3H). |
| 71 | 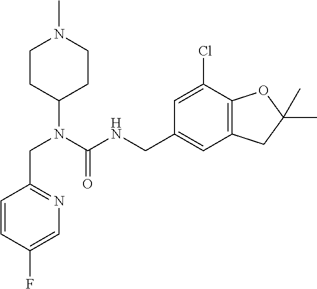 [SMILES: CN1CCC(N(Cc2ccc(F)cn2)C(=O)NCc2cc(Cl)c3c(c2)CC(C)(C)O3)CC1] | MS m/z (ESI): 461.1[M + 1], 483.0[M + 23] ¹H NMR (600 MHz, Methanol-d₄) δ 8.39 (d, 1H), 7.57 (d, 1H), 7.42 (m, 1H), 6.97 (d, 2H), 4.56 (s, 2H), 4.34-4.26 (m, 1H), 4.24 (s, 2H), 3.40-3.34 (m, 2H), 3.06 (s, 2H), 2.90 (s, 2H), 2.71 (s, 3H), 2.05-1.96 (m, 2H), 1.89-1.83 (m, 2H), 1.47 (s, 6H). |
| 72 | 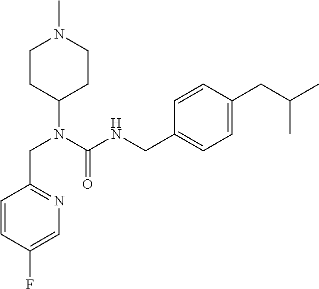 [SMILES: CC(C)Cc1ccc(CNC(=O)N(Cc2ccc(F)cn2)C2CCN(C)CC2)cc1] | MS m/z (ESI): 413.4[M + 1], 435.4[M + 23] ¹H NMR (600 MHz, DMSO-d₆) δ 8.48 (d, 1H), 7.68 (m, 1H), 7.35 (m, 1H), 7.16 (t, 1H), 7.11 (d, 2H), 7.06 (d, 2H), 4.48 (s, 2H), 4.26 (d, 1H), 4.23 (d, 2H), 3.28-3.16 (m, 2H), 2.82 (s, 2H), 2.57 (s, 3H), 2.40 (d, 2H), 2.06- 1.88 (m, 2H), 1.79 (m, 1H), 1.70-1.59 (m, 2H), 0.84 (d, 6H). |
| 75 | 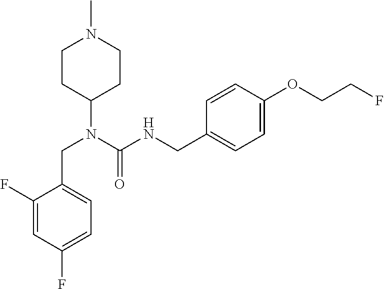 [SMILES: CN1CCC(N(Cc2ccc(F)cc2F)C(=O)NCc2ccc(OCCF)cc2)CC1] | MS m/z (ESI): 436.4[M + 1]¹H NMR (600 MHz, DMSO-d₆) δ 7.24-7.17 (m, 2H), 7.17-7.13 (m, 2H), 7.11 (t, 1H), 7.03 (m, 1H), 6.91-6.85 (m, 2H), 4.79- 4.74 (m, 1H), 4.70-4.67 (m, 1H), 4.41 (s, 2H), 4.24-4.21 (m, 1H), 4.19 (s, 2H), 4.17 (d, 2H), 3.23-3.08 (m, 2H), 2.51 (m, 3H), 2.49(s, 2H), 1.85 (s, 2H), 1.66- 1.59 (m, 2H). |
| 76 | 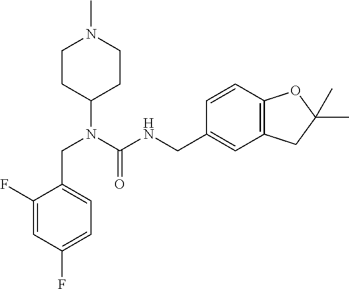 [SMILES: CN1CCC(N(Cc2ccc(F)cc2F)C(=O)NCc2ccc3c(c2)CC(C)(C)O3)CC1] | MS m/z (ESI): 444.3[M + 1]¹H NMR (600 MHz, DMSO-d₆) δ 7.25-7.15 (m, 2H), 7.03 (m, 2H), 6.99 (d, 1H), 6.94- 6.86 (m, 1H), 6.58 (d, 1H), 4.41 (s, 2H), 4.27-4.18 (m, 1H), 4.15 (d, 2H), 3.17 (d, 2H), 2.94 (s, 2H), 2.51 (m, 3H), 2.47 (m, 2H), 1.85 (d, 2H), 1.61 (d, 2H), 1.38 (s, 6H). |
| 77 | 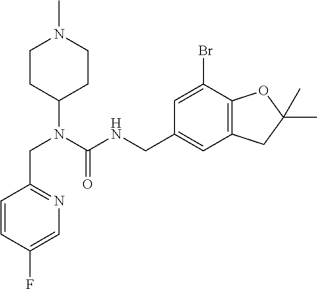 [SMILES: CN1CCC(N(Cc2ccc(F)cn2)C(=O)NCc2cc(Br)c3c(c2)CC(C)(C)O3)CC1] | MS m/z (ESI): 505.3[M + 1]¹H NMR (600 MHz, DMSO-d₆) δ 8.48 (d, 1H), 7.68 (m, 1H), 7.33 (m, 1H), 6.97 (d, 1H), 6.91 (m, 1H), 6.58 (d, 1H), 4.46 (s, 2H), 4.24-4.17 (m, 1H), 4.15 (d, 2H), 3.16 (d, 2H), 2.94 (s, 2H), 2.55-2.51 (m, 2H), 2.50 (m, 3H), 1.89 (s, 2H), 1.62 (d, 2H), 1.38 (s, 6H). |
| 80 | 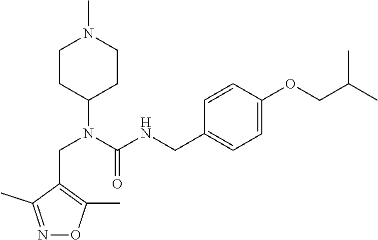 [SMILES: Cc1noc(C)c1CN(C(=O)NCc1ccc(OCC(C)C)cc1)C1CCN(C)CC1] | MS m/z (ESI): 429.2[M + 1]¹H NMR (600 MHz, CDCl₃) δ 7.16 (m, 2H), 6.89- 6.68 (m, 2H), 4.63 (t, 1H), 4.34 (d, 2H), 4.21-4.18 (m, 2H), 3.91(m, 1H), 3.69 (m, 2H), 2.88(m, 2H), 2.26 (s, 2H), 2.22 (s, 3H), 2.17 (s, 3H), 2.04 (m, 2H), 1.68 (d, 4H), 1.01 (d, 7H). |
| 81 | 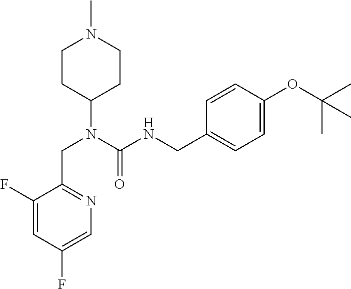 [SMILES: CN1CCC(N(Cc2ncc(F)cc2F)C(=O)NCc2ccc(OC(C)(C)C)cc2)CC1] | MS m/z (ESI): 447.0[M + 1]¹H NMR (600 MHz, CDCl₃) δ 8.11 (d, 1H), 7.22 (m, 1H), 7.19-7.14 (m, 2H), 7.04 (t, 1H), 6.95-6.89 (m, 2H), 4.49 (d, 2H), 4.38 (d, 3H), 3.24(s, 2H), 2.55 (s, 5H), 2.23 (s, 2H), 1.84-1.70 (m, 2H), 1.33 (s, 9H). |
| 83 | 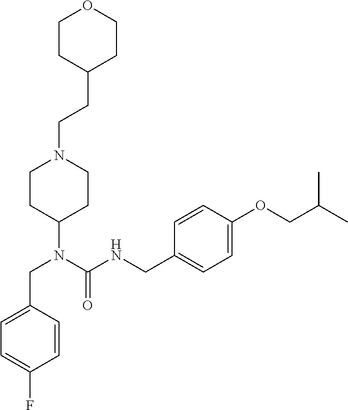 [SMILES: CC(C)COc1ccc(CNC(=O)N(Cc2ccc(F)cc2)C2CCN(CCC3CCOCC3)CC2)cc1] | MS m/z (ESI): 526.4[M + 1]¹H NMR (600 MHz, CDCl₃) δ 7.17 (m, 2H), 7.02- 6.91 (m, 4H), 6.77 (d, 2H), 4.51 (s, 2H), 4.37 (s, 2H), 4.26 (d, 2H), 3.98 (d, 2H), 3.96-3.90 (m, 1H), 3.67 (d, 2H), 3.35 (d, 3H), 2.73 (t, 1H), 2.57-2.42 (m, 1H), 2.11-1.95 (m, 1H), 1.86-1.72 (m, 3H), 1.62-1.53 (m, 5H), 1.48 (tq, 1H), 1.38-1.25 (m, 3H), 1.00 (d, 6H). |
| 84 | 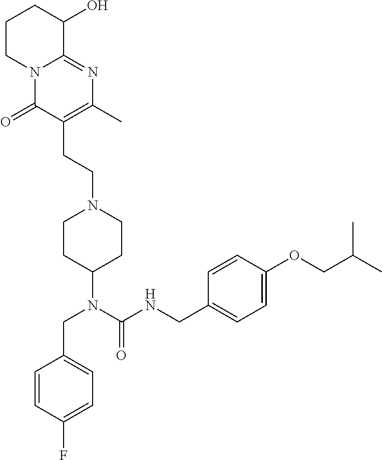 [SMILES: Cc1nc2n(c(=O)c1CCN1CCC(N(Cc3ccc(F)cc3)C(=O)NCc3ccc(OCC(C)C)cc3)CC1)CCCC2O] | MS m/z (ESI): 642.5[M + 1]¹H NMR (600 MHz, CDCl₃) δ 7.18 (dd, 2H), 7.02- 6.95 (m, 4H), 6.77 (d, 2H), 4.48 (dd, 2H), 4.38 (s, 2H), 4.27 (d, 2H), 4.07 (s, 1H), 3.98-3.83 (m, 2H), 3.68 (d, 2H), 3.39-2.49 (m, 6H), 2.35 (s, 5H), 2.12 (m, 1H), 2.06 (m, 1H), 2.00-1.91 (m, 1H), 1.88-1.79 (m, 2H), 1.75 (m, 2H), 1.65-1.47 (m, 1H), 1.26 (d, 1H), 1.01 (d, 6H). |
| 85 | 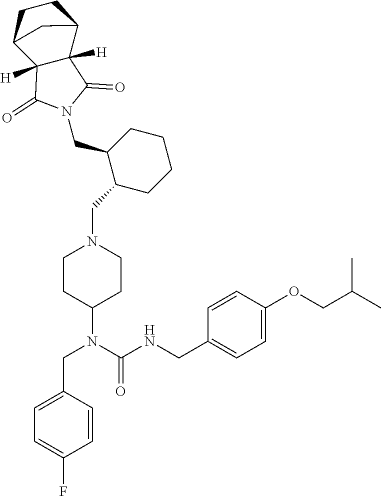 [SMILES: CC(C)COc1ccc(CNC(=O)N(Cc2ccc(F)cc2)C2CCN(C[C@H]3CCCC[C@@H]3CN3C(=O)[C@@H]4[C@H]5CC[C@H](C5)[C@@H]4C3=O)CC2)cc1] | MS m/z (ESI): 687.1[M + 1]¹H NMR (600 MHz, CDCl₃) δ 7.23-7.10 (m, 2H), 6.99 (td, 4H), 6.84-6.50 (m, 2H), 4.39 (d, 3H), 4.27 (d, 2H), 3.86-3.73 (m, 1H), 3.68 (d, 2H), 3.26 (s, 1H), 2.90 (d, 2H), 2.72-2.65 (m, 2H), 2.58 (s, 2H), 2.46 (s, 1H), 2.06 (m, 3H), 1.88- 1.51 (m, 9H), 1.48 (d, 2H), 1.39-1.30 (m, 2H), 1.29-1.07 (m, 6H), 1.01 (d, 6H), 0.98-0.83 (m, 2H). |
| 86 | 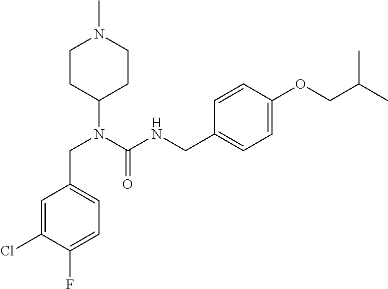 [SMILES: CC(C)COc1ccc(CNC(=O)N(Cc2ccc(F)c(Cl)c2)C2CCN(C)CC2)cc1] | MS m/z (ESI): 463.1[M + 1]¹H NMR (600 MHz, CDCl₃) δ 7.13-7.06 (m, 2H), 7.05-6.98 (m, 2H), 6.80 (d, 2H), 4.54-4.42 (m, 2H), 4.36 (s, 2H), 4.29 (d, 2H), 3.68 (d, 2H), 3.16 (d, 2H), 2.49 (s, 3H), 2.43 (s, 2H), 2.06 (s, 3H), 1.92- 1.71 (m, 2H), 1.01 (d, 6H). |
| 87 | 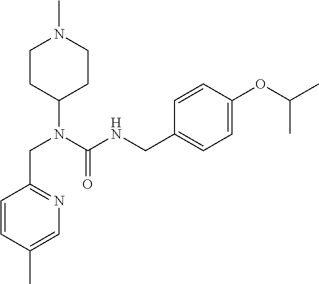 [SMILES: Cc1ccc(CN(C(=O)NCc2ccc(OC(C)C)cc2)C2CCN(C)CC2)nc1] | MS m/z (ESI): 411.3[M + 1]¹H NMR (600 MHz, CDCl₃) δ 8.22 (s, 1H), 7.45 (d, 1H), 7.18 (t, 3H), 6.81 (d, 2H), 4.51 (s, 1H), 4.34 (t, 5H), 2.99-2.90 (m, 2H), 2.31 (d, 6H), 2.19-2.10 (m, 2H), 1.91- 1.79 (m, 2H), 1.69 (d, 2H), 1.32 (d, 6H). |
| 88 | 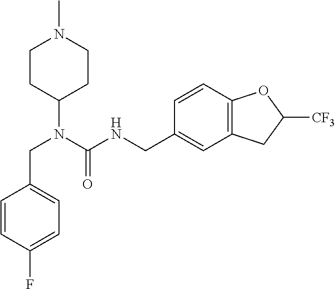 [SMILES: CN1CCC(N(Cc2ccc(F)cc2)C(=O)NCc2ccc3c(c2)CC(C(F)(F)F)O3)CC1] | MS m/z (ESI): 466.4[M + 1]¹H NMR (600 MHz, DMSO-d₆) δ 7.25 (m, 2H), 7.15-7.10 (m, 2H), 7.06 (s, 1H), 7.03- 6.97 (m, 1H), 6.90 (t, 1H), 6.82 (d, 1H), 5.46 (m, 1H), 4.41 (s, 2H), 4.19 (d, 2H), 4.00 (d, 1H), 3.53 (m, 1H), 3.23 (m, 1H), 2.93-2.85 (m, 2H), 2.26 (s, 5H), 1.70-1.60 (m, 2H), 1.50 (d, 2H). |
| 89 | 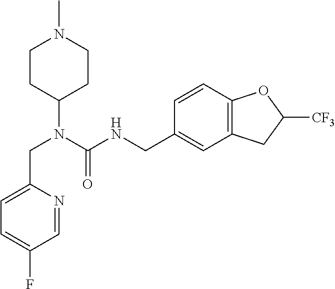 [SMILES: CN1CCC(N(Cc2ccc(F)cn2)C(=O)NCc2ccc3c(c2)CC(C(F)(F)F)O3)CC1] | MS m/z (ESI): 465.2 [M + 1]; ¹H NMR (400 MHz, CDCl₃) δ 8.26 (d, 1H), 7.39- 7.36 (m, 2H), 7.21-7.08 (m, 3H), 6.84- 6.81(m, 2H), 6.07-5.75 (m, 1H), 4.47- 4.32 (m, 5H), 4.03 (t, 2H), 3.03 (d, 2H), 2.41-2.15 (m, 5H), 2.11-1.96(m, 4H), 1.75-1.56(m, 4H). |
| 90 | 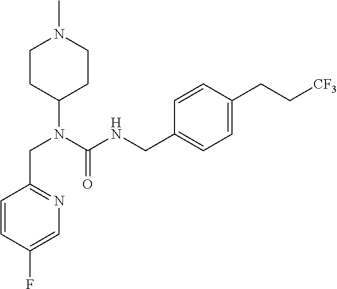 [SMILES: CN1CCC(N(Cc2ccc(F)cn2)C(=O)NCc2ccc(CCC(F)(F)F)cc2)CC1] | MS m/z (ESI): 453.5[M + 1]¹H NMR (600 MHz, DMSO-d₆) δ 8.48 (d, 1H), 7.68 (m, 1H), 7.33 (m, 1H), 7.20 (d, 2H), 7.14 (m, 3H), 4.48 (s, 2H), 4.23 (d, 2H), 4.11-4.02 (m, 1H), 2.98 (d, 2H), 2.81-2.73 (m, 2H), 2.55 (m, 2H), 2.35 (s, 5H), 1.71 (m, 2H), 1.55 (m, 2H). |
| 91 | 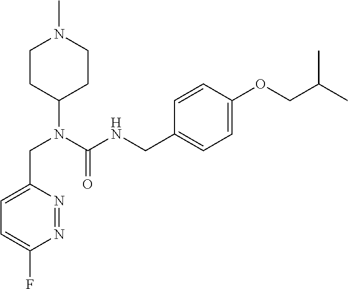 [SMILES: CC(C)COc1ccc(CNC(=O)N(Cc2ccc(F)nn2)C2CCN(C)CC2)cc1] | MS m/z (ESI): 430.3 [M + 1]; ¹H NMR (400 MHz, CDCl₃) δ 7.69-7.65 (m, 1H), 7.17-7.13 (m, 3H), 6.83-6.81 (m, 2H), 5.67-5.62(m, 1H), 4.65 (s, 2H), 4.31 (d, 2H), 4.10-4.01(m, 1H), 3.69(d, 2H), 2.88 (d, 2H), 2.26 (s, 3H), 2.14-1.97 (m, 3H), 1.76-1.66 (m, 4H), 1.01 (d, 6H). |
| 92 | 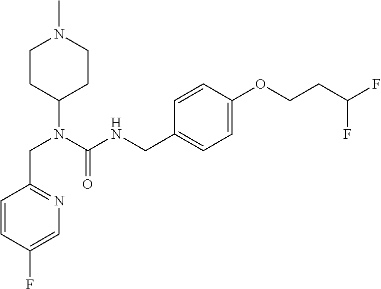 [SMILES: CN1CCC(N(Cc2ccc(F)cn2)C(=O)NCc2ccc(OCCC(F)F)cc2)CC1] | MS m/z (ESI): 451.2 [M + 1]; ¹H NMR (400 MHz, CDCl₃) δ 8.27 (d, 1H), 7.39- 7.36 (m, 2H), 7.21 (d, 2H), 6.86-6.82 (m, 3H), 6.24-5.96 (m, 1H), 4.42-4.33 (m, 5H), 4.11 (t, 2H), 3.02 (d, 2H), 2.38- 2.21 (m, 7H), 1.95-1.72(m, 4H). |
| 93 | 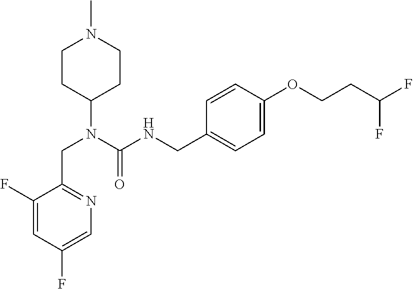 [SMILES: CN1CCC(N(Cc2ncc(F)cc2F)C(=O)NCc2ccc(OCCC(F)F)cc2)CC1] | MS m/z (ESI): 469.2 [M + 1]; ¹H NMR (400 MHz, CDCl₃) δ 8.16 (d, 1H), 7.25- 7.22 (m, 3H), 7.11-6.98 (m, 3H), 6.23- 5.95 (m, 1H), 4.42-4.23 (m, 4H), 4.19- 4.10 (m, 3H), 2.92 (d, 2H), 2.38-2.11 (m, 7H), 1.85-1.62(m, 4H). |
| 94 | 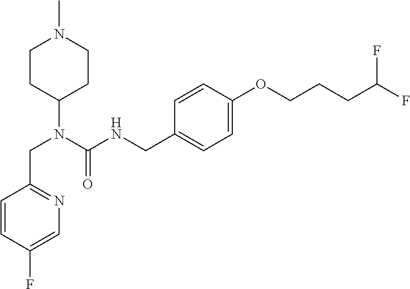 [SMILES: CN1CCC(N(Cc2ccc(F)cn2)C(=O)NCc2ccc(OCCCC(F)F)cc2)CC1] | MS m/z (ESI): 465.2 [M + 1]; ¹H NMR (400 MHz, CDCl₃) δ 8.26 (d, 1H), 7.39- 7.36 (m, 2H), 7.21-7.08 (m, 3H), 6.84- 6.81(m, 2H), 6.07-5.75 (m, 1H), 4.47- 4.32 (m, 5H), 4.03 (t, 2H), 3.03 (d, 2H), 2.41-2.15 (m, 5H), 2.11-1.96(m, 4H), 1.75-1.56(m, 4H). |
| 95 | 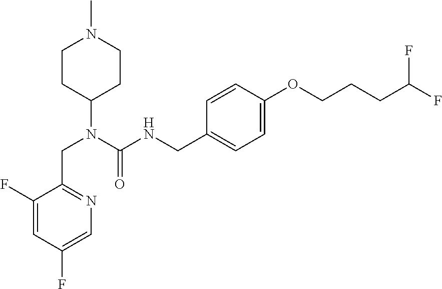 [SMILES: CN1CCC(N(Cc2ncc(F)cc2F)C(=O)NCc2ccc(OCCCC(F)F)cc2)CC1] | MS m/z (ESI): 483.2 [M + 1]; ¹H NMR (400 MHz, CDCl₃) δ 8.16 (d, 1H), 7.25- 7.22 (m, 3H), 7.11-6.98 (m, 3H), 6.23- 5.95 (m, 1H), 4.47-4.36 (m, 4H), 4.23- 4.03 (m, 3H), 2.93 (d, 2H), 2.31 (s, 3H), 2.11-1.96(m, 6H), 1.85-1.66(m, 4H). |
| 96 | 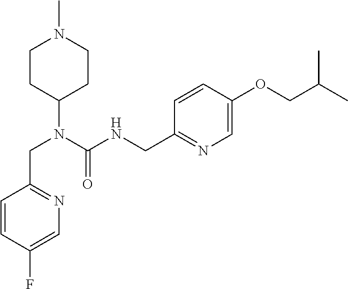 [SMILES: CC(C)COc1ccc(CNC(=O)N(Cc2ccc(F)cn2)C2CCN(C)CC2)nc1] | MS m/z (ESI): 430.2 [M + 1]; ¹H NMR (400 MHz, DMSO-d₆) δ 8.48 (d, 1H), 8.16 (d, 1H), 7.69-7.66 (m, 1H), 7.37- 7.34 (m, 2H), 7.19-7.15 (m, 2H), 4.48(s, 2H), 4.28 (d, 2H), 3.95-3.79(m, 3H), 2.72 (d, 2H), 2.13 (s, 3H), 2.04-1.87 (m, 3H), 1.61-1.46 (m, 4H), 0.95 (d, 6H). |
| 97 | 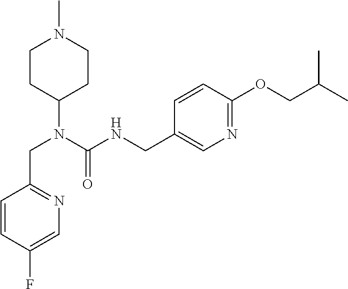 [SMILES: CC(C)COc1ccc(CNC(=O)N(Cc2ccc(F)cn2)C2CCN(C)CC2)cn1] | MS m/z (ESI): 430.2 [M + 1]; ¹H NMR (600 MHz, CDCl₃) δ 8.30-8.23 (m, 1H), 8.06 (d, 1H), 7.69 (s, 1H), 7.53 (dd, 1H), 7.11 (d, 2H), 6.69 (d, 1H), 4.41-4.32 (m, 5H), 4.03 (d, 2H), 3.08 (d, 2H), 2.43 (s, 3H), 2.34-2.27 (m, 2H), 2.11-2.02 (m, 3H), 1.69 (d, 2H), 1.01 (d, 6H). |
| 98 | 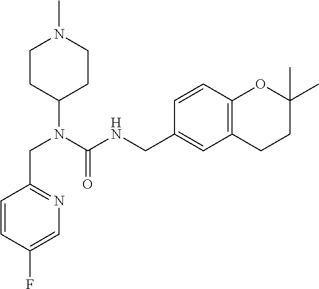 [SMILES: CN1CCC(N(Cc2ccc(F)cn2)C(=O)NCc2ccc3c(c2)CCC(C)(C)O3)CC1] | MS m/z (ESI): 441.3 [M + 1]; ¹H NMR (600 MHz, CDCl₃) δ 8.25 (d, 1H), 7.48- 7.35 (m, 2H), 7.15-7.08 (m, 1H), 7.02- 6.93 (m, 2H), 6.71 (d, 1H), 4.51-4.38 (m, 3H), 4.30 (d, 2H), 3.24-3.14 (m, 2H), 2.73 (t, 2H), 2.51 (s, 5H), 2.29- 2.16 (m, 2H), 1.81-1.68 (m, 4H), 1.31 (s, 6H). |
| 99 | 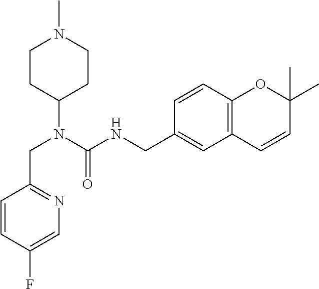 [SMILES: CN1CCC(N(Cc2ccc(F)cn2)C(=O)NCc2ccc3c(c2)C=CC(C)(C)O3)CC1] | MS m/z (ESI): 439.3 [M + 1]; ¹H NMR (600 MHz, DMSO-d₆) δ 8.47 (d, 1H), 7.75-7.60 (m, 2H), 7.34 (m, 1H), 7.04 (d, 1H), 6.95 (m, 1H), 6.91-6.84 (m, 1H), 6.63 (m, 1H), 6.34 (d, 1H), 4.47 (s, 2H), 4.14 (m, 3H), 3.05 (d, 2H), 2.50 (m, 3H), 2.41 (s, 2H), 1.75 (m, 2H), 1.62-1.50 (m, 2H), 1.30 (d, 6H). |
| 100 | 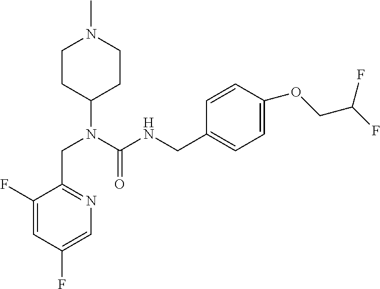 [SMILES: CN1CCC(N(Cc2ncc(F)cc2F)C(=O)NCc2ccc(OCC(F)F)cc2)CC1] | MS m/z (ESI): 454.3 [M + 1]; ¹H NMR (600 MHz, CDCl₃) δ 8.14 (d, 1H), 7.26- 7.20 (m, 3H), 7.10-7.04 (m, 1H), 6.90- 6.85 (m, 2H), 6.08 (tt, 1H), 4.48 (d, 2H), 4.39-4.30 (m, 3H), 4.17 (td, 2H), 3.12 (d, 2H), 2.46 (s, 3H), 2.36 (s, 2H), 1.74- 1.68 (m, 2H). |
| 101 | 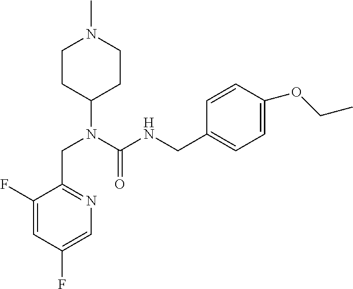 | MS m/z (ESI): 437.2 [M + 1]; ¹H NMR (600 MHz, CDCl₃) δ 8.14 (d, 1H), 7.25- 7.20 (m, 3H), 7.04 (t, 1H), 6.90-6.86 (m, 2H), 4.81-4.77 (m, 1H), 4.73-4.70 (m, 1H), 4.48 (d, 2H), 4.36 (d, 3H), 4.21 (ddd, 2H), 3.29-3.14 (m, 2H), 2.51 (d, 5H), 2.19 (s, 2H), 1.77-1.71(m, 2H). |

### Example 2

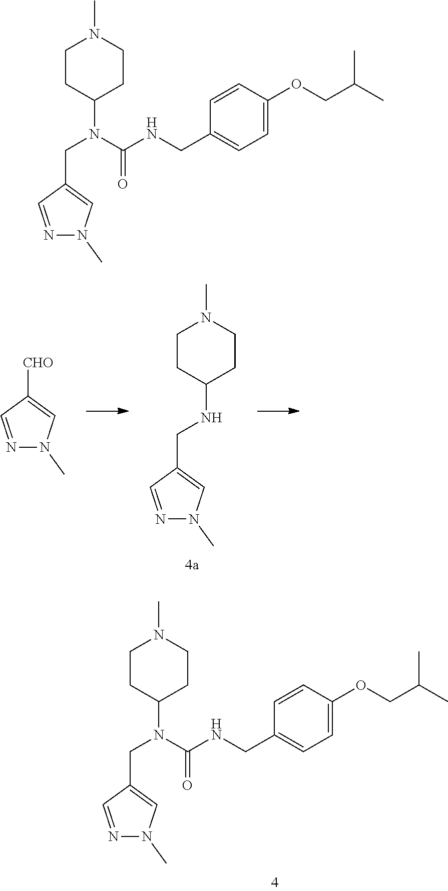

Under nitrogen protection, in an ice-water bath, 1-methylpyrazole-4-carbaldehyde (440 mg, 4.0 mmol) was dissolved in 10 ml of methanol. N-methyl-4-piperidone (452 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 4a (617 mg).

Under nitrogen protection, compound 4a (208 mg, 1.0 mmol) was dissolved in 5 ml of acetonitrile. N-(4-isobutyloxybenzyl)-1H-imidazol-formamide (273 mg, 1.0 mmol) and potassium carbonate (207 mg, 1.5 mmol) were added, and the resulting mixture was heated to 60° C. and reacted under stirring for 12 h. The reaction solution was cooled to room temperature and filtered. 10 ml of water was added to the filtrate, and then the resulting solution was extracted with dichloromethane (5 mL*3). The organic phases were combined, dried over anhydrous sodium sulfate, filtered, concentrated under reduced pressure and separated by column chromatography (dichloromethane:methanol=10:1) to obtain compound 4 (172 mg, yield: 43%). MS m/z (ESI): 414.3 [M+1]; ¹H NMR (400 MHz, CDCl₃) δ 7.38 (s, 1H), 7.17 (s, 1H), 7.12-7.07 (m, 2H), 6.82-6.77 (m, 2H), 4.69 (m, 1H), 4.30-4.25 (m, 2H), 4.23-4.20 (m, 2H), 3.78 (s, 3H), 3.69-3.64 (d, 2H), 2.98-2.88 (m, 2H), 2.32 (s, 3H), 2.18-2.02 (m, 3H), 1.80-1.65 (m, 4H), 1.04-0.99 (d, 6H).

Compounds 5, 8 and 22 were prepared in a similar manner to compound 4.

TABLE 2
Structures and characterization data of compounds 5, 8 and 22

| Compound | | |
|---|---|---|
| number | Structural formula | Characterization data |
| 5 | 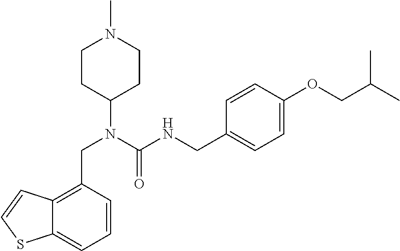 [SMILES: CC(C)COc1ccc(CNC(=O)N(Cc2cccc3sccc23)C2CCN(C)CC2)cc1] | MS m/z (ESI): 466.2 [M + 1]; ¹H NMR(400 MHZ, CDCl₃) δ 7.79(d, 1H), 7.52(d, 1H), 7.40-7.37(m, 1H), 7.30- 7.27(m, 1H), 7.25-7.23(m, 1H), 7.02- 6.98(m, 2H), 6.78-6.75(m, 2H), 4.75(s, 3H), 4.68-4.59(m, 2H), 4.26(d, 2H), 3.70(d, 2H), 3.20-3.05(m, 3H), 2.50- 2.34(m, 4H), 2.10-1.98(m, 2H), 1.89- 1.82(m, 2H), 1.41-1.35(m, 2H), 1.02- 0.98(d, 6H). |
| 8 | 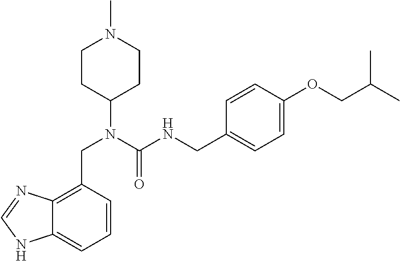 [SMILES: CC(C)COc1ccc(CNC(=O)N(Cc2cccc3[nH]cnc23)C2CCN(C)CC2)cc1] | MS m/z (ESI): 450.3 [M + 1]; ¹H NMR(400 MHZ, CDCl₃) δ 7.82(d, 1H), 7.51(d, 1H), 7.20-7.09(m, 5H), 6.78- 6.73(m, 2H), 4.75(s, 2H), 4.28(d, 2H), 3.68(d, 2H), 3.20-3.05(m, 2H), 2.50- 2.34(m, 5H), 2.15-1.95 (m, 4H), 1.69- 1.52(m, 2H), 1.00-0.95(d, 6H). |
| 22 | 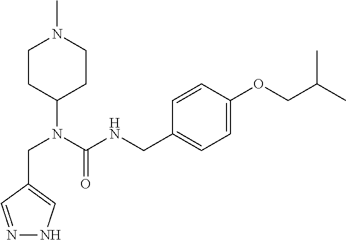 [SMILES: CC(C)COc1ccc(CNC(=O)N(Cc2cn[nH]c2)C2CCN(C)CC2)cc1] | MS m/z (ESI): 400.3 [M + 1]; ¹H NMR(400 MHZ, CDCl₃) δ 7.42(s, 2H), 7.12-7.06(m, 2H), 6.84-6.79(m, 2H), 4.73(m, 1H), 4.41(m, 1H), 4.31- 4.25(m, 4H), 3.70-3.66(d, 2H), 3.18- 3.10(m, 2H), 2.47(s, 3H), 2.44-2.34(m, 2H), 2.10-1.98(m, 3H), 1.80-1.72(m, 2H), 1.03-0.98(d, 6H). |

### Example 3

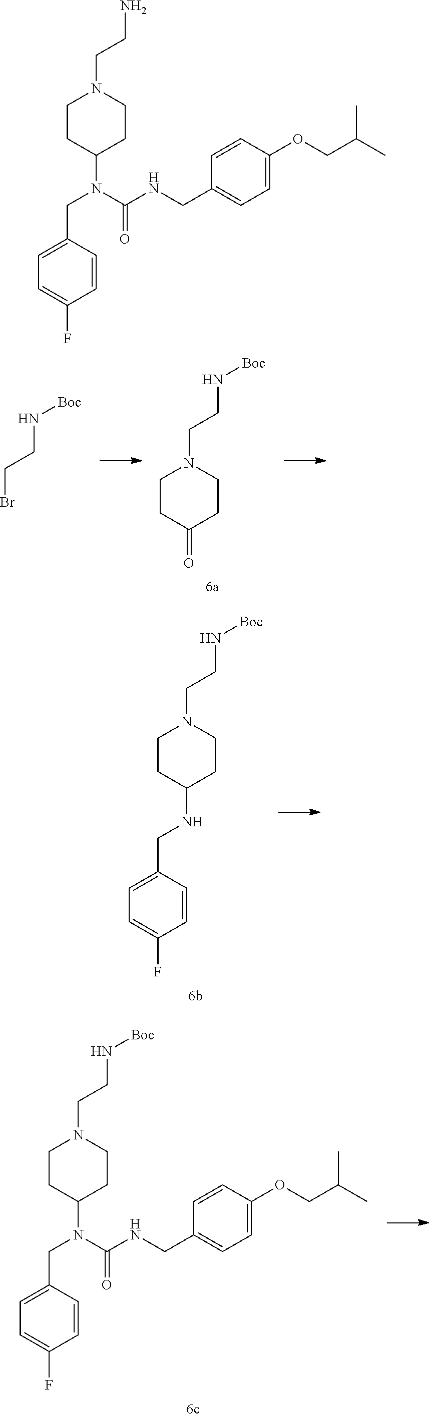 [SMILES: CC(C)COc1ccc(CNC(=O)N(Cc2ccc(F)cc2)C2CCN(CCN)CC2)cc1 ; CC(C)(C)OC(=O)NCCBr ; CC(C)(C)OC(=O)NCCN1CCC(=O)CC1 ; CC(C)(C)OC(=O)NCCN1CCC(NCc2ccc(F)cc2)CC1 ; CC(C)COc1ccc(CNC(=O)N(Cc2ccc(F)cc2)C2CCN(CCNC(=O)OC(C)(C)C)CC2)cc1]

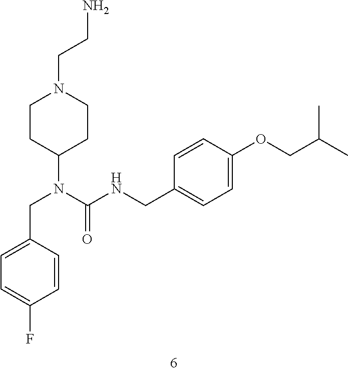 [SMILES: CC(C)COc1ccc(CNC(=O)N(Cc2ccc(F)cc2)C2CCN(CCN)CC2)cc1]

N-Boc-bromoethylamine (2.23 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 mL of water was added, and then the resulting solution was extracted with dichloromethane (60 mL*3). The organic phases were combined, dried over anhydrous sodium sulfate, filtered and concentrated under reduced pressure to obtain compound 6a (1.97 g).

Under nitrogen protection, in an ice-water bath, 4-fluorobenzylamine (500 mg, 4.0 mmol) was dissolved in 10 ml of methanol. Compound 6a (968 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 6b (912 mg).

Under nitrogen protection, compound 6b (702 mg, 2.0 mmol) was dissolved in 10 ml of acetonitrile. N-(4-isobutyloxybenzyl)-1H-imidazol-formamide (546 mg, 2.0 mmol) and potassium carbonate (414 mg, 3.0 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 6c (467 mg).

Under nitrogen protection, compound 6c (278 mg, 0.5 mmol) was dissolved in 5 mL of dichloromethane. Trifluoroacetic acid (285 mg, 2.5 mmol) was added, and the resulting mixture was reacted at room temperature for 2 h. An aqueous solution of NaHCO₃ was added to adjust the pH value to alkaline. The organic phase was concentrated and then extracted with dichloromethane (5 ml*3). The organic phases were combined, dried over anhydrous sodium sulfate, filtered and concentrated under reduced pressure to obtain compound 6 (201 mg, yield: 88%). MS m/z (ESI): 457.2 [M+1]; ¹H NMR (400 MHz, CDCl₃) δ 7.25-7.23 (m, 2H), 7.02-6.98 (m, 4H), 6.78-6.75 (m, 2H), 4.48-4.27 (m, 5H), 3.62-3.59 (m, 2H), 3.51-3.29 (m, 2H), 2.94-2.89 (m, 2H), 2.84-2.80 (m, 2H), 2.47-2.41 (m, 2H), 2.18-2.02 (m, 3H), 1.75-1.68 (m, 4H), 1.02-0.98 (d, 6H).

### Example 4

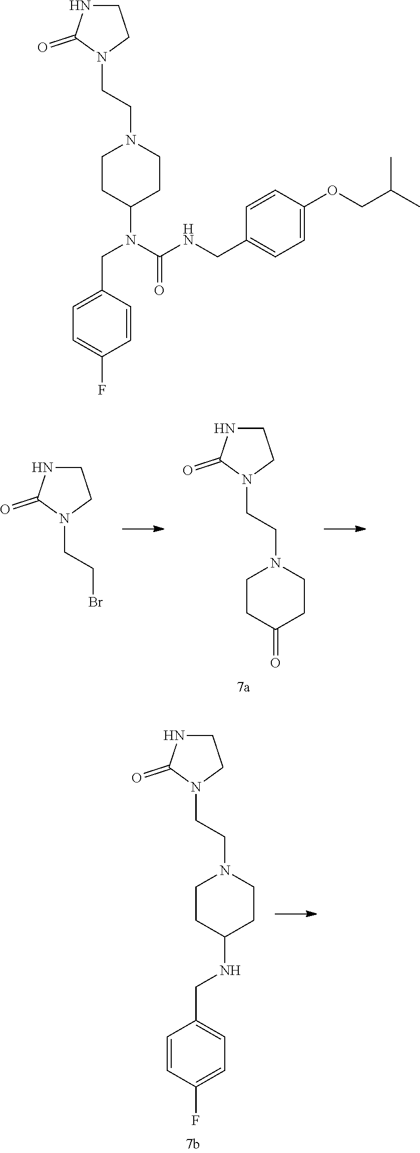 [SMILES: CC(C)COc1ccc(CNC(=O)N(Cc2ccc(F)cc2)C2CCN(CCN3CCNC3=O)CC2)cc1 ; O=C1NCCN1CCBr ; O=C1CCN(CCN2CCNC2=O)CC1 ; O=C1NCCN1CCN1CCC(NCc2ccc(F)cc2)CC1]

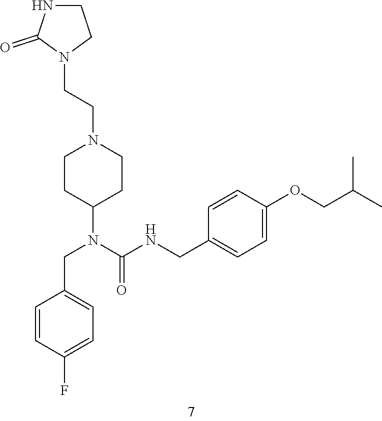 [SMILES: CC(C)COc1ccc(CNC(=O)N(Cc2ccc(F)cc2)C2CCN(CCN3CCNC3=O)CC2)cc1]

1-(2-Bromoethyl)imidazolidine-2-one (1.93 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 ml of water was added, and then the resulting solution was extracted with dichloromethane (60 mL*3). The organic phases were combined, dried over anhydrous sodium sulfate, filtered and concentrated under reduced pressure to obtain compound 7a (1.56 g).

Under nitrogen protection, in an ice-water bath, 4-fluorobenzylamine (500 mg, 4.0 mmol) was dissolved in 10 ml of methanol. Compound 7a (844 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 7b (812 mg).

Under nitrogen protection, compound 7b (640 mg, 2.0 mmol) was dissolved in 10 ml of acetonitrile. N-(4-isobutyloxybenzyl)-1H-imidazol-formamide (546 mg, 2.0 mmol) and potassium carbonate (414 mg, 3.0 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 7 (494 mg, yield: 47%). MS m/z (ESI): 526.3 [M+1]; ¹H NMR (400 MHz, CDCl₃) δ 7.24-7.22 (m, 2H), 7.02-6.98 (m, 4H), 6.78-6.75 (m, 2H), 4.60-4.54 (m, 2H), 4.32-4.26 (m, 4H), 3.72-3.62 (m, 4H), 3.51-3.44 (m, 2H), 3.41-3.32 (m, 2H), 3.30-3.26 (m, 2H), 2.98-2.92 (m, 2H), 2.47-2.41 (m, 2H), 2.18-2.02 (m, 3H), 1.74-1.65 (m, 4H), 1.02-0.99 (d, 6H).

### Example 5

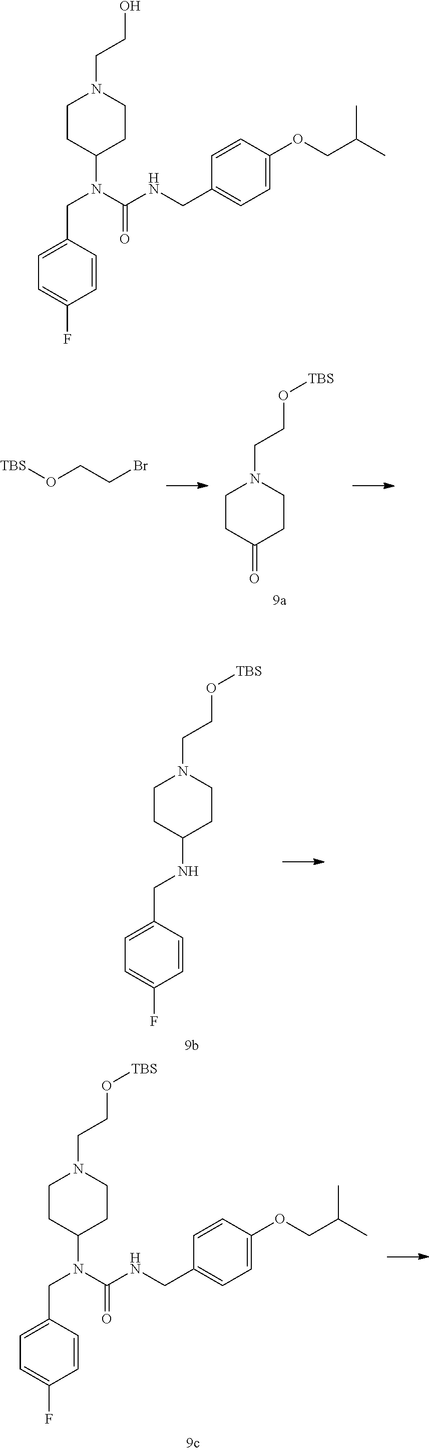

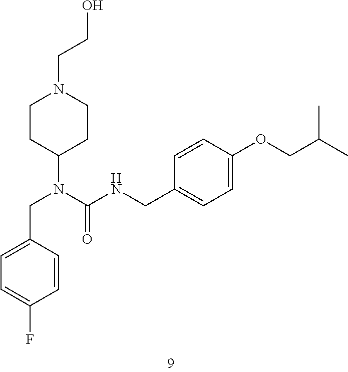 [SMILES: CC(C)COc1ccc(CNC(=O)N(Cc2ccc(F)cc2)C2CCN(CCO)CC2)cc1]

Tert-butyl dimethyl bromoethoxysilane (2.38 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 ml of water was added, and then the resulting solution was extracted with dichloromethane (60 mL*3). The organic phases were combined, dried over anhydrous sodium sulfate, filtered and concentrated under reduced pressure to obtain compound 9a (1.72 g).

Under nitrogen protection, in an ice-water bath, 4-fluorobenzylamine (500 mg, 4.0 mmol) was dissolved in 10 ml of methanol. Compound 9a (1028 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 9b (812 mg).

Under nitrogen protection, compound 9b (732 mg, 2.0 mmol) was dissolved in 10 ml of acetonitrile. N-(4-isobutyloxybenzyl)-1H-imidazol-formamide (546 mg, 2.0 mmol) and potassium carbonate (414 mg, 3.0 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 9c (327 mg).

Under nitrogen protection, compound 9c (286 mg, 0.5 mmol) was dissolved in 5 ml of THF. A 1 M tetrabutylammonium fluoride tetrahydrofuran solution (1 ml, 1.0 mmol) was added, and the resulting mixture was reacted at room temperature for 2 h. The organic phase was concentrated and then extracted with dichloromethane (5 mL*3). The organic phases were combined, dried over anhydrous sodium sulfate, filtered and concentrated under reduced pressure to obtain compound 9 (118 mg, yield: 52%). MS m/z (ESI): 458.3 [M+1]; H NMR (400 MHz, CDCl₃) δ 7.23-7.20 (m, 2H), 7.02-6.98 (m, 4H), 6.81-6.76 (m, 2H), 4.70-4.50 (m, 2H), 4.38-4.27 (m, 4H), 3.78-3.69 (m, 4H), 3.41-3.19 (m, 2H), 2.84-2.74 (m, 2H), 2.67-2.51 (m, 2H), 2.18-2.02 (m, 2H), 1.81-1.58 (m, 3H), 1.02-0.98 (d, 6H).

### Example 6

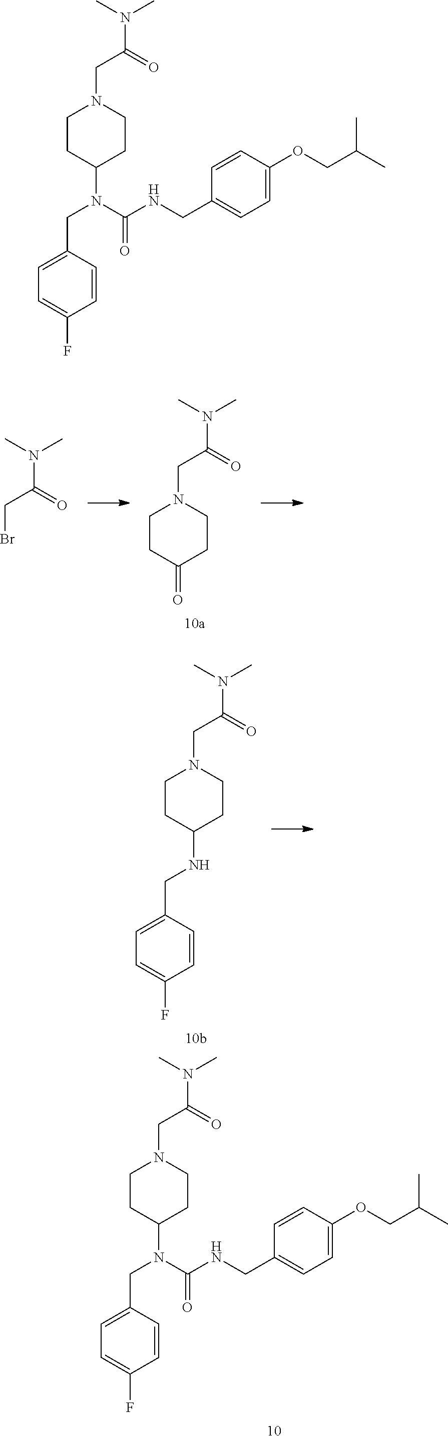

2-Bromo-N,N-dimethylacetamide (1.66 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 ml of water was added, and then the resulting solution was extracted with dichloromethane (60 mL*3). The organic phases were combined, dried over anhydrous sodium sulfate, filtered and concentrated under reduced pressure to obtain compound 10a (1.15 g).

Under nitrogen protection, in an ice-water bath, 4-fluorobenzylamine (500 mg, 4.0 mmol) was dissolved in 10 ml of methanol. Compound 10a (736 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 10b (832 mg).

Under nitrogen protection, compound 10b (586 mg, 2.0 mmol) was dissolved in 10 ml of acetonitrile. N-(4-isobutyloxybenzyl)-1H-imidazol-formamide (546 mg, 2.0 mmol) and potassium carbonate (414 mg, 3.0 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 10 (535 mg, yield: 54%). MS m/z (ESI): 499.3 [M+1]; ¹H NMR (400 MHz, CDCl₃) δ 7.20-7.13 (m, 2H), 7.02-6.95 (m, 4H), 6.77-6.73 (m, 2H), 4.48-4.43 (m, 1H), 4.38-4.27 (m, 3H), 4.26-4.23 (m, 2H), 3.66 (d, 2H), 3.15 (d, 2H), 3.02-2.88 (m, 8H), 2.30-2.20 (m, 2H), 2.10-2.00 (m, 1H), 1.80-1.65 (m, 4H), 1.01-0.96 (d, 6H).

### Example 7

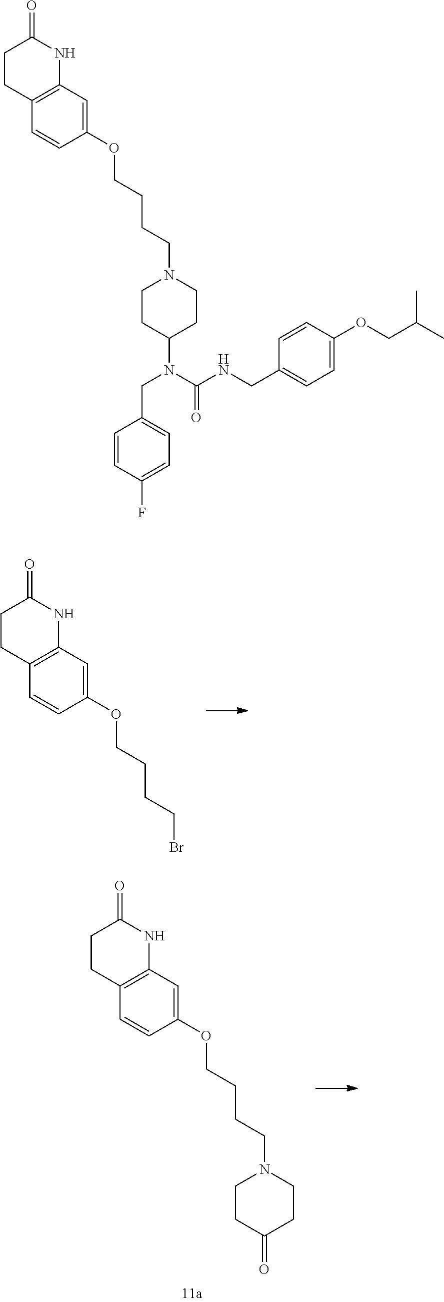 [SMILES: CC(C)COc1ccc(CNC(=O)N(Cc2ccc(F)cc2)C2CCN(CCCCOc3ccc4c(c3)NC(=O)CC4)CC2)cc1 ; O=C1CCc2ccc(OCCCCBr)cc2N1 ; O=C1CCN(CCCCOc2ccc3c(c2)NC(=O)CC3)CC1]

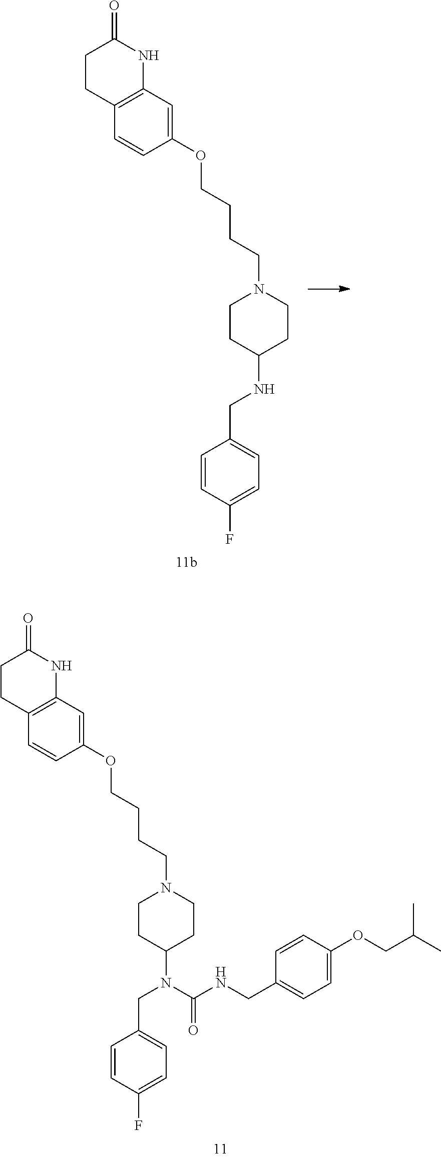 [SMILES: O=C1CCc2ccc(OCCCCN3CCC(NCc4ccc(F)cc4)CC3)cc2N1 ; CC(C)COc1ccc(CNC(=O)N(Cc2ccc(F)cc2)C2CCN(CCCCOc3ccc4c(c3)NC(=O)CC4)CC2)cc1]

7-(4-Bromobutoxy)-3,4-dihydro-2(1H)-quinolinone (2.98 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 ml of water was added, and then the resulting solution was extracted with dichloromethane (60 mL*3). The organic phases were combined, dried over anhydrous sodium sulfate, filtered and concentrated under reduced pressure to obtain compound 11a (2.26 g).

Under nitrogen protection, in an ice-water bath, 4-fluorobenzylamine (500 mg, 4.0 mmol) was dissolved in 10 ml of methanol. Compound 11a (1.26 g, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 11b (1.03 g).

Under nitrogen protection, compound 11b (425 mg, 1.0 mmol) was dissolved in 10 ml of acetonitrile. N-(4-isobutyloxybenzyl)-1H-imidazol-formamide (273 mg, 1.0 mmol) and potassium carbonate (207 mg, 1.5 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 11 (311 mg, yield: 49%). MS m/z (ESI): 631.3 [M+1]; ¹H NMR (400 MHz, CDCl₃) δ 7.96 (s, 1H), 7.20-7.15 (m, 2H), 7.04-6.96 (m, 4H), 6.79-6.75 (m, 2H), 6.50-6.43 (m, 1H), 6.32-6.28 (m, 1H), 4.70-4.52 (m, 2H), 4.48-4.43 (m, 2H), 4.31-4.27 (m, 2H), 3.98-3.90 (m, 2H), 3.64 (d, 2H), 3.50-3.20 (d, 2H), 2.82-2.58 (m, 4H), 2.56-2.44 (m, 4H), 2.10-2.00 (m, 2H), 1.90-1.65 (m, 7H), 1.02-0.97 (d, 6H).

### Example 8

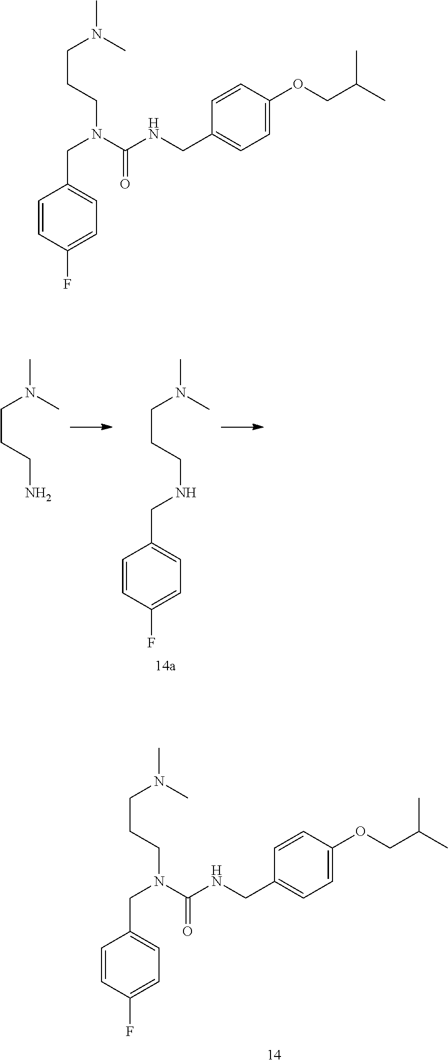

Under nitrogen protection, in an ice-water bath, 4-fluorobenzylamine (500 mg, 4.0 mmol) was dissolved in 10 ml of methanol. N,N-dimethyl-1,3-diaminopropane (408 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 14a (587 mg).

Under nitrogen protection, compound 14a (210 mg, 1.0 mmol) was dissolved in 5 ml of acetonitrile. N-(4-isobutyloxybenzyl)-1H-imidazol-formamide (273 mg, 1.0 mmol) and potassium carbonate (207 mg, 1.5 mmol) were added, and the resulting mixture was heated to 60° C. and reacted under stirring for 12 h. The reaction solution was cooled to room temperature and filtered. 10 ml of water was added to the filtrate, and then the resulting solution was extracted with dichloromethane (5 ml*3). The organic phases were combined, dried over anhydrous sodium sulfate, filtered, concentrated under reduced pressure and separated by column chromatography (dichloromethane:methanol=10:1) to obtain compound 13 (202 mg, yield: 48%). MS m/z (ESI): 416.3 [M+1]; ¹H NMR (400 MHz, CDCl₃) δ 7.26-7.20 (m, 4H), 7.02-6.97 (m, 2H), 6.81-6.76 (m, 2H), 4.47 (s, 2H), 4.38-4.35 (m, 2H), 3.71 (s, 2H), 3.30-3.24 (m, 2H), 2.35-2.25 (m, 2H), 2.15-2.00 (m, 7H), 1.65-1.58 (m, 2H), 1.02-0.98 (d, 6H).

### Example 9

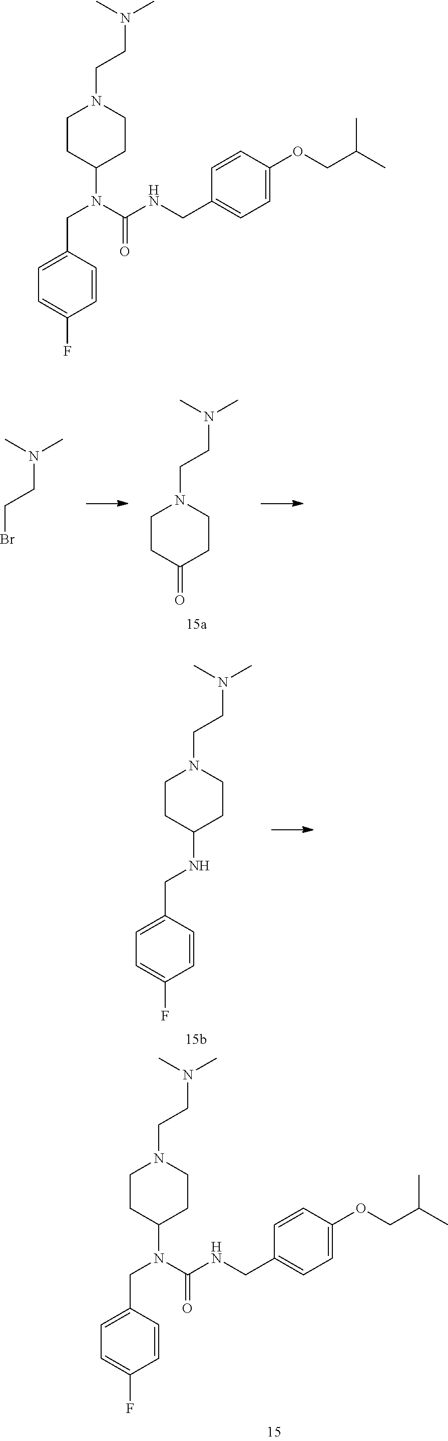

(2-Bromomethyl)dimethylamine (1.52 g, 10.0 mmol) was dissolved in 20 ml of DMF. 4-Piperidone (0.99 g, 10.0 mmol) and potassium carbonate (2.07 g, 15.0 mmol) were added, and the resulting mixture was heated to 80° C. and reacted under stirring for 8 h. The reaction solution was cooled to room temperature. 60 ml of water was added, and then the resulting solution was extracted with dichloromethane (60 mL*3). The organic phases were combined, dried over anhydrous sodium sulfate, filtered and concentrated under reduced pressure to obtain compound 15a (1.17 g).

Under nitrogen protection, in an ice-water bath, 4-fluorobenzylamine (500 mg, 4.0 mmol) was dissolved in 10 ml of methanol. Compound 15a (680 mg, 4.0 mmol) and sodium triacetoxyborohydride (933 mg, 4.4 mmol) were added, and the resulting mixture was heated to room temperature and reacted for 15 h. An aqueous solution of NaHCO₃ was added to adjust the pH value to alkaline. The organic phase was concentrated and then extracted with dichloromethane (10 ml*3). The organic phases were combined, dried over anhydrous sodium sulfate, filtered and concentrated under reduced pressure to obtain compound 15b (611 mg).

Under nitrogen protection, compound 15b (279 mg, 1.0 mmol) was dissolved in 10 ml of acetonitrile. N-(4-isobutyloxybenzyl)-1H-imidazol-formamide (273 mg, 1.0 mmol) and potassium carbonate (207 mg, 1.5 mmol) were added, and the resulting mixture was heated to 60° C. and reacted under stirring for 12 h. The reaction solution was cooled to room temperature and filtered. 20 mL of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 15 (287 mg, yield: 59%). MS m/z (ESI): 485.3 [M+1]; ¹H NMR (400 MHz, CDCl₃) δ 7.22-7.16 (m, 2H), 7.02-6.95 (m, 4H), 6.77-6.75 (m, 2H), 4.55-4.46 (m, 1H), 4.38-4.32 (m, 2H), 4.26-4.23 (m, 2H), 3.67 (d, 2H), 3.19 (d, 1H), 2.98-2.68 (m, 6H), 2.61 (s, 6H), 2.45-2.35 (m, 2H), 2.10-2.00 (m, 2H), 1.80-1.65 (m, 2H), 1.01-0.96 (d, 6H).

### Example 10

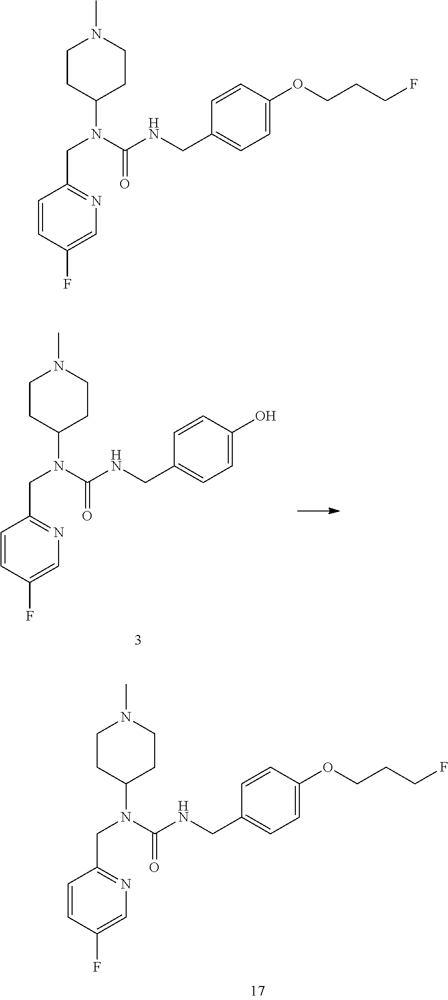 [SMILES: CN1CCC(N(Cc2ccc(F)cn2)C(=O)NCc2ccc(OCCCF)cc2)CC1 ; CN1CCC(N(Cc2ccc(F)cn2)C(=O)NCc2ccc(O)cc2)CC1 ; CN1CCC(N(Cc2ccc(F)cn2)C(=O)NCc2ccc(OCCCF)cc2)CC1]

Under nitrogen protection, compound 3 (372 mg, 1.0 mmol) was dissolved in 10 ml of acetonitrile. 1-Bromo-3-fluoropropane (211 mg, 1.5 mmol) and cesium carbonate (652 mg, 2.0 mmol) were added, and the resulting mixture was heated to 60° C. and reacted under stirring for 5 h. The reaction solution was cooled to room temperature and filtered. 10 ml of water was added to the filtrate, and then the resulting solution was extracted with dichloromethane (10 ml*3). The organic phases were combined, dried over anhydrous sodium sulfate, filtered, concentrated under reduced pressure and separated by column chromatography (dichloromethane:methanol=10:1) to obtain compound 17 (238 mg, yield: 54%). MS m/z (ESI): 433.2 [M+1]; ¹H NMR (400 MHz, CDCl₃) δ 8.22 (s, 1H), 7.45-7.35 (m, 2H), 7.22-7.14 (m, 2H), 7.15-7.07 (m, 1H), 6.85-6.81 (m, 2H), 4.67 (t, 1H), 4.60 (t, 1H), 4.44-4.34 (m, 3H), 4.34 (d, 2H), 4.07 (t, 2H), 3.12 (d, 2H), 2.46 (s, 3H), 2.40-2.32 (m, 2H), 2.19-2.05 (m, 4H), 1.75-1.67 (m, 2H).

Compounds 18, 55-57, 59-60, 62, 65, 69, 73-74, 78 and 82 were prepared in a similar manner to compound 17.

TABLE 3
Structures and characterization data of compoundes 18, 55-57, 59-60, 62, 65, 69, 73-74, 78 and 82

| Compound | | |
|---|---|---|
| number | Structural formula | Characterization data |
| 18 | 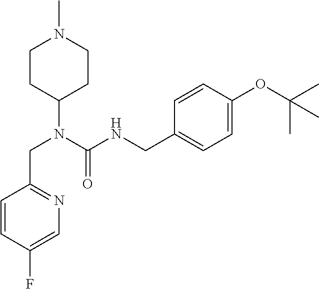 [SMILES: CN1CCC(N(Cc2ccc(F)cn2)C(=O)NCc2ccc(OC(C)(C)C)cc2)CC1] | MS m/z (ESI): 429.3 [M + 1]; ¹H NMR(400 MHz,CDCl₃) δ 8.23(s, 1H), 7.39-7.34(m, 2H), 7.17-7.14(m, 2H), 6.98-6.90(m, 3H), 4.37(s, 2H), 4.41(s, 2H), 4.38-4.34(m, 2H), 3.06-3.00(m, 2H), 2.39(s, 3H), 2.30-2.24(m, 2H), 2.02-1.93(m, 2H), 1.73-1.68(m, 2H), 1.32(s, 9H). |
| 55 | 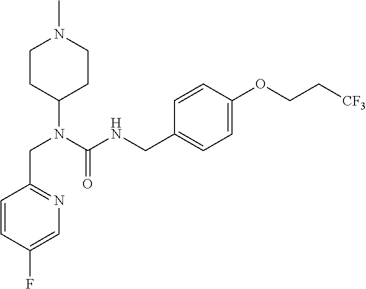 [SMILES: CN1CCC(N(Cc2ccc(F)cn2)C(=O)NCc2ccc(OCCC(F)(F)F)cc2)CC1] | MS m/z (ESI): 469.2 [M + 1]; 1H NMR(400 MHz, CDCl3) δ 8.27(d, 1H), 7.44-7.35(m, 2H), 7.25-7.19(m, 2H), 7.17-7.08(m, 1H), 6.85-6.80(m, 2H), 4.42-4.34(m, 5H), 4.18(t, 2H), 3.10(d, 2H), 2.65-2.58(m, 2H), 2.45(s, 3H), 2.37-2.30(m, 2H), 2.11-2.02(m, 2H), 1.73-1.67(m, 2H). |
| 56 | 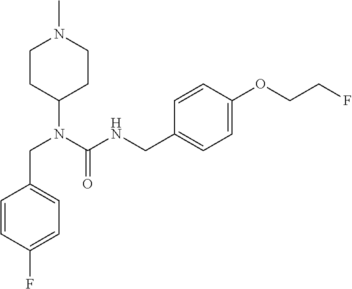 [SMILES: CN1CCC(N(Cc2ccc(F)cc2)C(=O)NCc2ccc(OCCF)cc2)CC1] | MS m/z (ESI): 418.2 [M + 1]; 1H NMR(400 MHz, CDCl3) δ 7.20-7.15(m, 2H), 7.04- 6.94(m, 4H), 6.85-6.77(m, 2H), 4.77(t, 1H), 4.68(t, 1H), 4.54-4.38(m, 2H), 4.34(s, 2H), 4.27(d, 2H), 4.22-4.14(m, 2H), 3.02(d, 2H), 2.36(s, 3H), 2.30-2.18(m, 2H), 1.95-1.73(m, 4H). |
| 57 | 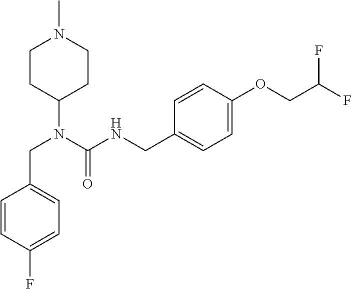 [SMILES: CN1CCC(N(Cc2ccc(F)cc2)C(=O)NCc2ccc(OCC(F)F)cc2)CC1] | MS m/z (ESI): 436.2 [M + 1]; 1H NMR(400 MHz, CDCl3) δ 7.21-7.15(m, 2H), 7.05- 6.96(m, 4H), 6.83-6.77(m, 2H), 6.15- 5.97(m, 1H), 4.55(t, 1H), 4.50-4.35(m, 3H), 4.28(d, 2H), 4.15(dt, 2H), 3.03(d, 2H), 2.38(s, 3H), 2.31-2.22(m, 2H), 1.92-1.73(m, 4H). |
| 59 | 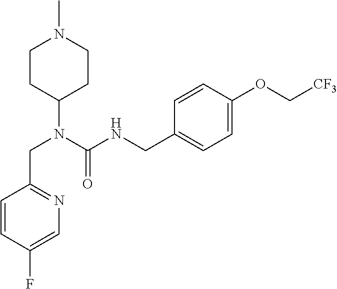 [SMILES: CN1CCC(N(Cc2ccc(F)cn2)C(=O)NCc2ccc(OCC(F)(F)F)cc2)CC1] | MS m/z (ESI): 455.5[M + 1], 477.5[M + 23]1H NMR (600 MHz, DMSO-d6) δ 8.49 (d, 1H), 7.74-7.65 (m, 1H), 7.33 (m, 1H), 7.23-7.17 (m, 2H), 7.13 (s, 1H), 7.00-6.95 (m, 2H), 4.72 (q, 2H), 4.47 (s, 2H), 4.21 (d, 2H), 4.16- 4.05 (m, 1H), 3.07 (s, 2H), 2.50 (d, 3H), 2.44 (s, 2H), 1.77 (d, 2H), 1.58 (d, 2H). |
| 60 | 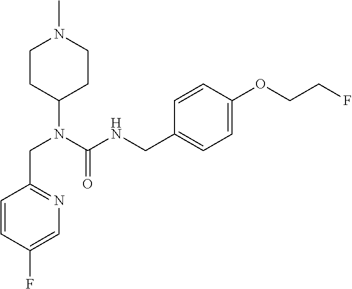 [SMILES: CN1CCC(N(Cc2ccc(F)cn2)C(=O)NCc2ccc(OCCF)cc2)CC1] | MS m/z (ESI): 419.48[M + 1]1H NMR (600 MHz, CDCl3) δ 8.24 (d, 1H), 7.52 (m, 1H), 7.40 (m, 2H), 7.23 (d, 2H), 6.88 (d, 2H), 4.81-4.77 (m, 1H), 4.72-4.69 (m, 1H), 4.52- 4.45 (m, 1H), 4.43 (s, 2H), 4.36 (d, 2H), 4.25-4.21 (m, 1H), 4.20-4.16 (m, 1H), 3.29- 3.23 (m, 2H), 2.58 (s, 3H), 2.56 (s, 2H), 2.33 (d, 2H), 1.74 (m, 2H). |
| 62 | 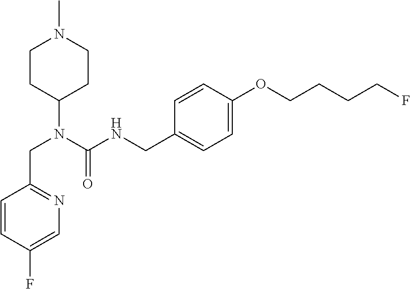 [SMILES: CN1CCC(N(Cc2ccc(F)cn2)C(=O)NCc2ccc(OCCCCF)cc2)CC1] | MS m/z (ESI): 447.26[M + 1]1H NMR (600 MHz, DMSO-d6) δ 8.48 (d, 1H), 7.68 (d, 1H), 7.33 (m, 1H), 7.16-7.07 (m, 3H), 6.85 (d, 2H), 4.54 (t, 1H), 4.46 (q, 3H), 4.18 (d, 2H), 3.97 (t, 1H), 3.09 (s, 2H), 2.50 (m, 3H), 2.47-2.41 (m, 2H), 1.86-1.73 (m, 6H), 1.62- 1.54 (m, 2H), 1.29-1.19 (m, 2H). |
| 65 | 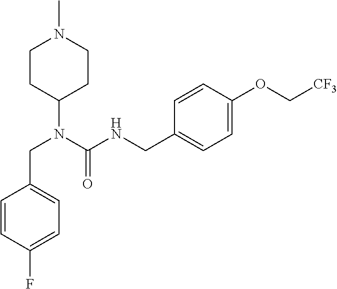 [SMILES: CN1CCC(N(Cc2ccc(F)cc2)C(=O)NCc2ccc(OCC(F)(F)F)cc2)CC1] | MS m/z (ESI): 454.2[M + 1], 476.3[M + 23]¹H NMR (600 MHZ, DMSO-d₆) δ 7.24 (m, 2H), 7.19-7.15 (m, 2H), 7.14-7.10 (m, 2H), 6.98 (m, 3H), 4.72 (q, 2H), 4.41 (s, 2H), 4.20 (d, 2H), 4.04 (s, 1H), 2.93 (s, 2H), 2.50 (t, 3H), 2.30 (s, 2H), 1.82-1.65 (m, 2H), 1.50 (d, 2H). |
| 69 | 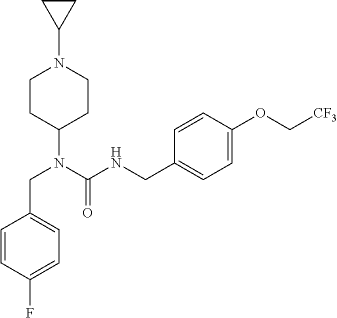 [SMILES: O=C(NCc1ccc(OCC(F)(F)F)cc1)N(Cc1ccc(F)cc1)C1CCN(C2CC2)CC1] | MS m/z (ESI): 480.4[M + 1]¹H NMR (600 MHz, DMSO-d₆) δ 7.25-7.20 (m, 2H), 7.19- 7.15 (m, 2H), 7.14-7.07 (m, 2H), 7.00-6.96 (m, 2H), 6.93 (t, 1H), 4.72 (q, 2H), 4.39 (s, 2H), 4.20 (d, 2H), 3.96 (s, 1H), 2.88 (d, 2H), 2.17 (d, 2H), 1.43 (d, 4H), 1.24 (d, 1H), 0.36 (d, 2H), 0.21 (s, 2H). |
| 73 | 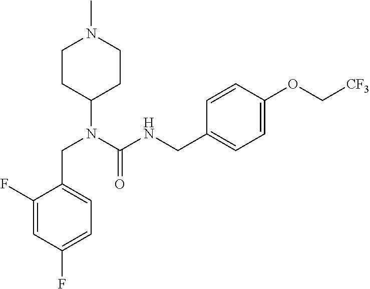 [SMILES: CN1CCC(N(Cc2ccc(F)cc2F)C(=O)NCc2ccc(OCC(F)(F)F)cc2)CC1] | MS m/z (ESI): 472.2[M + 1]¹H NMR (600 MHz, DMSO-d₆) δ 7.24-7.15 (m, 4H), 7.15- 7.08 (m, 1H), 7.03 (m, 1H), 6.97 (d, 2H), 4.72 (d, 2H), 4.40 (s, 2H), 4.19 (d, 2H), 4.14- 4.04 (m, 1H), 2.97 (s, 2H), 2.50 (m, 3H), 2.34 (s, 2H), 1.71 (s, 2H), 1.56 (d, 2H). |
| 74 | 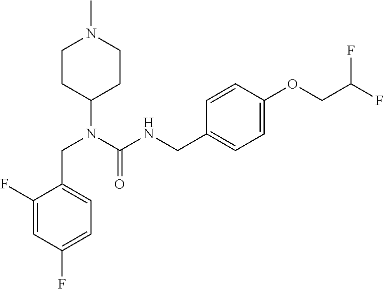 [SMILES: CN1CCC(N(Cc2ccc(F)cc2F)C(=O)NCc2ccc(OCC(F)F)cc2)CC1] | MS m/z (ESI): 454.1[M + 1]¹H NMR (600 MHz, DMSO-d₆) δ 7.24-7.18 (m, 2H), 7.16 (d, 2H), 7.13 (s, 1H), 7.04 (td, 1H), 6.93 (d, 2H), 6.38 (m, 1H), 4.41 (s, 2H), 4.28 (d, 2H), 4.19 (d, 3H), 3.21-3.08 (m, 2H), 2.64 (d, 2H), 2.51 (p, 3H), 1.94-1.80 (m, 2H), 1.62 (d, 2H). |
| 78 | 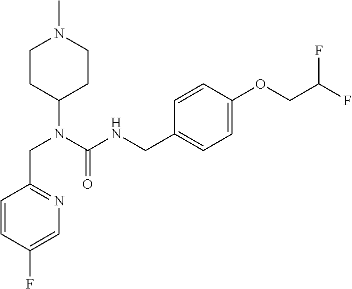 [SMILES: CN1CCC(N(Cc2ccc(F)cn2)C(=O)NCc2ccc(OCC(F)F)cc2)CC1] | MS m/z (ESI): 437.2[M + 1]¹H NMR (600 MHz, CDCl₃) δ 8.23 (d, 1H), 7.56 (m, 2H), 7.40 (m, 1H), 7.26-7.22 (m, 2H), 6.88-6.82 (m, 2H), 6.07 (s, 1H), 4.50 (t, 1H), 4.43 (s, 2H), 4.36 (d, 2H), 4.16 (m, 2H), 3.37-3.29 (m, 2H), 2.67(s, 2H), 2.64 (s, 2H), 2.43 (m, 3H), 1.78-1.66 (m, 2H). |
| 82 | 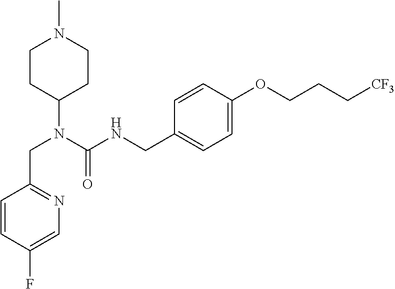 [SMILES: CN1CCC(N(Cc2ccc(F)cn2)C(=O)NCc2ccc(OCCCC(F)(F)F)cc2)CC1] | MS m/z (ESI): 483.2[M + 1]¹H NMR (600 MHz, DMSO-d₆) δ 8.48 (d, 1H), 7.77-7.57 (m, 1H), 7.33 (m, 1H), 7.21-7.08 (m, 3H), 6.96-6.82 (m, 2H), 4.46 (s, 2H), 4.18 (d, 2H), 4.16-4.12 (m, 1H), 4.00 (t, 2H), 3.07 (s, 2H), 2.50 (m, 3H), 2.48-2.36 (m, 2H), 1.96-1.88 (m, 2H), 1.87-1.75 (m, 2H), 1.58 (d, 2H), 1.42-1.21 (m, 2H). |

### Example 11

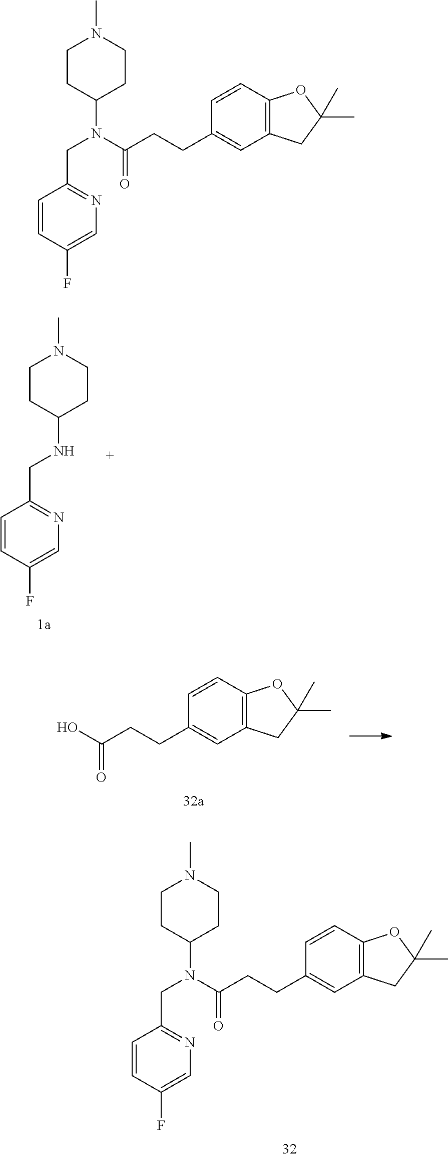

Under nitrogen protection, compound 1a (446 mg, 2.0 mmol) was dissolved in 10 ml of acetonitrile. Compound 32a (440 mg, 2.0 mmol), HATU (760 mg, 2.0 mmol) and diisopropylethylamine (387 mg, 3.0 mmol) were added, and the resulting mixture was heated to 60° C. and reacted under stirring for 12 h. The reaction solution was cooled to room temperature and filtered. 20 ml of water was added to the filtrate, and then the resulting solution was extracted with dichloromethane (10 mL*3). The organic phases were combined, dried over anhydrous sodium sulfate, filtered, concentrated under reduced pressure and separated by column chromatography (dichloromethane:methanol=10:1) to obtain compound 32 (561 mg, yield: 66%). MS m/z (ESI): 426.2 [M+1]; ¹H NMR (400 MHz, DMSO-d₆) δ 8.50 (s, 1H), 7.72-7.64 (m, 1H), 7.26-7.20 (m, 1H), 6.99-6.87 (m, 2H), 6.62-6.53 (m, 1H), 4.53 (s, 2H), 4.38-4.24 (m, 1H), 2.97-2.92 (d, 2H), 2.80-2.61 (m, 5H), 2.55-2.52 (m, 1H), 2.12 (s, 3H), 1.98-1.88 (m, 2H), 1.65-1.30 (m, 10H).

Compounds 53 and 54 were prepared in a similar manner to compound 32.

TABLE 4
Structures and characterization data of compounds 53 and 54

| Compound | | |
|---|---|---|
| number | Structural formula | Characterization data |
| 53 | 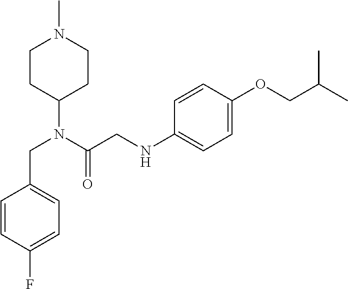 [SMILES: CC(C)COc1ccc(NCC(=O)N(Cc2ccc(F)cc2)C2CCN(C)CC2)cc1] | MS m/z (ESI): 428.3 [M + 1]; ¹H NMR(400 MHZ, DMSO-d₆) δ 7.34- 7.27(m, 2H), 7.18-7.09(m, 2H), 6.75- 6.68(m, 2H), 6.62-6.50(m, 2H), 4.99(s, 1H), 4.55(s, 2H), 3.96-3.76(m, 3H), 3.63(m, 2H), 2.72(d, 2H), 2.18(s, 3H), 2.03-1.85(m, 3H), 1.75-1.53(m, 4H), 0.92(d, 6H). |
| 54 | 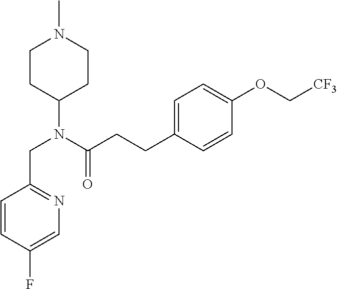 [SMILES: CN1CCC(N(Cc2ccc(F)cn2)C(=O)CCc2ccc(OCC(F)(F)F)cc2)CC1] | MS m/z (ESI): 454.2 [M + 1]; ¹H NMR(400 MHZ, DMSO-d₆) δ 8.5(d, 1H), 7.74-7.67(m, 1H), 7.38-7.19(m, 2H), 7.12-7.07(m, 1H), 6.99-6.88(m, 2H), 4.75-4.65(m, 2H), 4.60-4.50(m, 2H), 4.40-4.31(m, 1H), 3.25-3.15(m, 2H), 2.90-2.75(m, 5H), 2.65-2.55(m, 4H), 1.85-1.75(m, 2H), 1.70-1.58(m, 2H). |

### Example 12

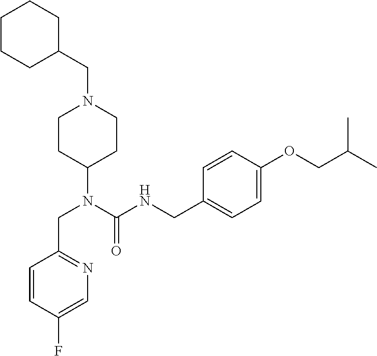 [SMILES: CC(C)COc1ccc(CNC(=O)N(Cc2ccc(F)cn2)C2CCN(CC3CCCCC3)CC2)cc1]

### Synthesis Route:

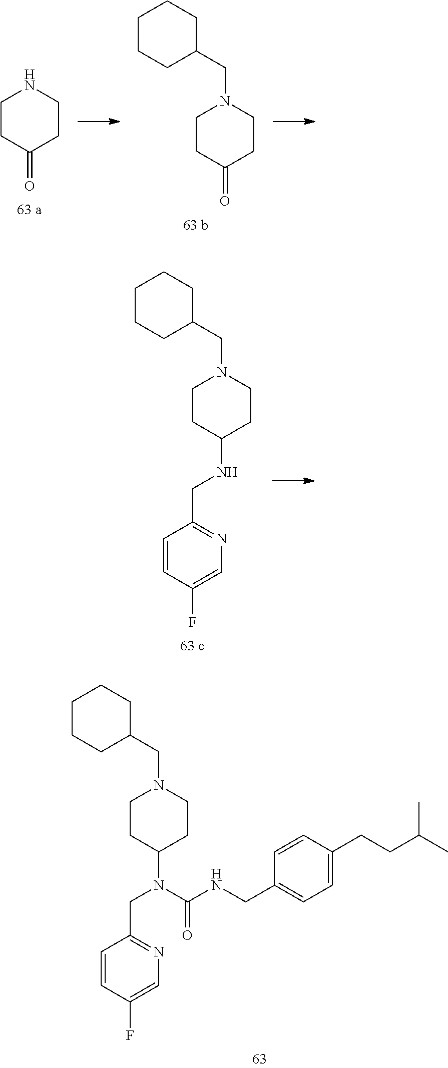 [SMILES: O=C1CCNCC1 ; O=C1CCN(CC2CCCCC2)CC1 ; Fc1ccc(CNC2CCN(CC3CCCCC3)CC2)nc1 ; CC(C)CCc1ccc(CNC(=O)N(Cc2ccc(F)cn2)C2CCN(CC3CCCCC3)CC2)cc1]

Under nitrogen protection, compound 63a (1.212 g, 11.3 mmol) was dissolved in 10 ml of acetonitrile. Bromomethyl cyclohexane (2 g, 11.3 mmol) and potassium carbonate (4.68 g, 33.9 mmol) were added, and the resulting mixture was heated to 60° C. and reacted under stirring for 12 h. The reaction solution was cooled to room temperature and filtered, and then the filtrate was extracted with dichloromethane (50 mL*3). The organic phases were combined, dried over anhydrous sodium sulfate, filtered, concentrated under reduced pressure and separated by column chromatography (dichloromethane:methanol=10:1) to obtain compound 63b (1.49 g, a light yellow oily liquid).

Under nitrogen protection, compound 63b (1.49 g, 7.63 mmol) was dissolved in 20 ml of methanol. (5-Fluoropyridin-2-methyl)amine (962.4 mg, 7.63 mmol) and sodium triacetoxyborohydride (1.972 g, 9.31 mmol) were added, and the resulting mixture was heated to room temperature and reacted for 15 h. An aqueous solution of NaHCO₃ was added to adjust the pH value to alkaline. The organic phase was concentrated and then extracted with dichloromethane (50 mL*3). The organic phases were combined, dried over anhydrous sodium sulfate, filtered, concentrated under reduced pressure and subjected to column chromatography to obtain compound 63c (217 mg).

Under nitrogen protection, compound 63c (217 mg, 0.71 mmol) was dissolved in 10 ml of acetonitrile. N-(4-isobutyloxybenzyl)-1H-imidazol-1-formamide (194 mg, 0.710 mmol) and potassium carbonate (107.9 mg, 0.781 mmol) were added, and the resulting mixture was heated to 60° C. and reacted under stirring for 12 h. The reaction solution was cooled to room temperature and filtered, and then the filtrate was extracted with dichloromethane (30 mL*3). The organic phases were combined, dried over anhydrous sodium sulfate, filtered, concentrated under reduced pressure and separated by column chromatography (dichloromethane:methanol=10:1) to obtain compound 63 (50.5 mg, a light yellow oily liquid, yield: 14%). MS m/z (ESI): 511.69[M+1]¹H NMR (600 MHz, CDCl₃) δ 8.24 (d, 1H), 7.37 (d, 2H), 7.17 (d, 2H), 6.90-6.75 (m, 2H), 4.42 (s, 2H), 4.33 (d, 2H), 3.70 (d, 2H), 3.11 (s, 2H), 2.41-2.14 (m, 1H), 2.11-2.01 (m, 2H), 1.85-1.63 (m, 10H), 1.46-1.36 (m, 2H), 1.31 (d, 2H), 1.24 (d, 2H), 1.16 (s, 2H), 1.02 (s, 3H), 1.01 (s, 3H).

### Example 13

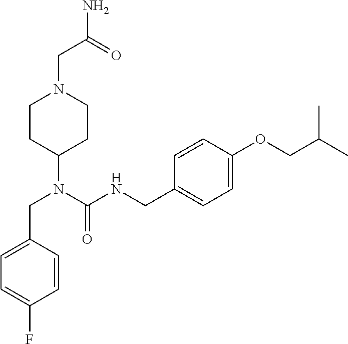 [SMILES: CC(C)COc1ccc(CNC(=O)N(Cc2ccc(F)cc2)C2CCN(CC(N)=O)CC2)cc1]

### Synthesis Route:

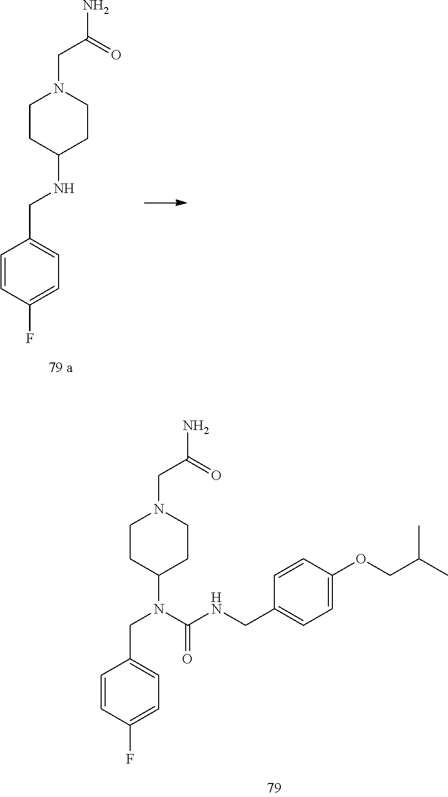 [SMILES: NC(=O)CN1CCC(NCc2ccc(F)cc2)CC1 ; CC(C)COc1ccc(CNC(=O)N(Cc2ccc(F)cc2)C2CCN(CC(N)=O)CC2)cc1]

Under nitrogen protection, compound 79a (200 mg, 0.75 mmol) was dissolved in 10 ml of acetonitrile. N-(4-isobutyloxybenzyl)-1H-imidazol-1-formamide (207 mg, 0.75 mmol) and potassium carbonate (105 mg, 0.75 mmol) were added, and the resulting mixture was heated to 60° C. and reacted under stirring for 5 h. The reaction solution was cooled to room temperature and filtered, and then the filtrate was extracted with dichloromethane (20 mL*3). The organic phases were combined, dried over anhydrous sodium sulfate, filtered, concentrated under reduced pressure and separated by column chromatography (dichloromethane:methanol=10:1) to obtain compound 79 (166 mg, a light yellow oily liquid, yield: 47%). MS m/z (ESI): 471.2[M+1]¹H NMR (600 MHz, CDCl₃) δ 7.20 (m, 2H), 7.01 (m, 4H), 6.92 (s, 1H), 6.81-6.74 (m, 2H), 5.40 (s, 1H), 4.44 (t, 1H), 4.34 (s, 3H), 4.27 (d, 2H), 3.68 (d, 2H), 2.98 (s, 2H), 2.94-2.87 (m, 2H), 2.31 (m, 2H), 2.06 (m, 1H), 1.80-1.74 (m, 2H), 1.66 (m, 2H), 1.01 (d, J=6.7 Hz, 6H).

### Test Example 1. In-Vitro Activity Test of 5-HT_{2A} Receptor

### 1. Screening for 5-HT_{2A} Inverse Agonistic Activity

### 1.1 Test Materials:

Cell lines: Adherent cells NIH3T3-5-HT_{2A} R
Cell culture medium: DMEM+10% FBS (purchased from GBICO)
Cell culture plate: White-wall clear-bottom 96-well plate (purchased from Perkin Elmer)
Detection kit: Bright-Glo™ Luciferase (purchased from Promega)
Detection instrument: BioTek multifunctional microplate reader

### 1.2 Test Drugs

Pimavanserin: Purchased from MCE Corporation
Other compounds: Prepared according to the preceding examples

### 1.3 Test Method:

NIH₃T3-5HT_{2A}R cells in the logarithmic growth phase were seeded into a white-wall clear-bottom 96-well plate at a density of 1000 cells per well, and cultured overnight in a 37° C., 5% CO₂ incubator. The next day, a compound to be tested was added to the cells, wherein the compound to be tested was subjected to 3.16-fold gradient dilution with PBS to obtain 9 concentrations, with the highest concentration of 10 uM, and double replicate wells were set up for each concentration; and PBS was used as a negative control group, and pimavanserin with the same concentration was used as a positive control group. After the addition, the cells were cultured in a 37° C., 5% CO₂ incubator for another 120 h. On the sixth day, a Bright-Glo™ Luciferase reagent with a volume equal to that of cell sap was added to the cells, and the cells were incubated at room temperature in the dark for 20 min. The plate was shaken every 5 min with a plate shaker, the luminescent intensity was detected by a microplate reader, and the cell inhibition rate was calculated. The data was processed with GraphPad Prism 7.0 to obtain a cell inhibition rate curve, and the IC₅₀ was calculated. The test results were as shown in Table 5.

Cell inhibition rate (%)=[100−(Lum test drug−Lum culture solution)/(Lum cell control−Lum culture solution)×100]%

### 2. Test for 5-HT_{2A} Antagonistic Activity (i.e., Calcium Ion Antagonism)

### 2.1 Test Materials:

Cell lines: CHO-K1/5-HT_{2A} (Chempartner)
Cell culture medium: DMEM/F12+10% FBS (purchased from GBICO)
Cell culture plate: 384-well Assay Plate (purchased from Corning)
Detection kit: FLIPR® Calcium 4 Assay Kit (purchased from Molecular Devices)

### 2.2 Test Drugs

Pimavanserin: Purchased from MCE Corporation
Other compounds: Prepared according to the preceding examples

### 2.3 Test Method:

CHO-K1/5-HT_{2A} cells in the logarithmic growth phase were seeded into a 384-well plate at a density of 10000 cells per well, and cultured in a 37° C., 5% CO₂ incubator for 16-24 h. After that, centrifugation was performed to remove the culture medium in the cell culture plate. Dye was added, and the cells were incubated in a 37° C., 5% CO₂ incubator for another 1 h. The cell culture plate was placed on the FLIPR. A compound to be tested was added, and the Ca²⁺ signal was detected, wherein the compound to be tested was subjected to 3-fold gradient dilution with PBS to obtain 10 concentrations, with the highest concentration of 10 uM, and double replicate wells were set up. 100 nM a-methyl-5-HT was added 15 min later, and the Ca²⁺ signal was detected again, wherein the value obtained by only adding 100 nM a-methyl-5-HT was taken as the maximum signal. In order to determine the test stability, risperidone was used as a positive control. The data were processed with GraphPad Prism 7.0 to obtain a cell inhibition rate curve, and the IC₅₀ was calculated. The test results were as shown in Table 5.

Inhibition rate (%)=100%−[(signal value of the compound to be tested−signal value of the test solution)/(the maximum signal value−the signal value of the test solution)]×100%

### Test Example 2. hERG Inhibitory Activity

### 1. Test Materials and Instruments

### 1.1 Positive Control Compound

Name: Cisapride

### 1.2 Solvent

Name: Dimethyl sulfoxide (DMSO)

### 1.3 Cells

Species & strains: CHO-hERG cell lines (Chinese hamster ovary cells stably expressing hERG channels)
Culture medium: 90% F12, 10% fetal bovine serum, 100 μg/mL G418 and 100 μg/mL hygromycin B
Culture conditions: 5% CO2, 37° C. incubator
Freezing conditions: Liquid nitrogen

### 1.4 Test Instruments

Patch clamp amplifier (Axoclamp 200B, Multiclamp 700B, Axon, US)
Digital analog converter (DigiData 1440A, DigiData 1550B, Axon, US)
Inverted microscope (IX51, IX71, Olympus, Japan)
Rapid drug delivery system (RSC-200, Bio-Logic, France)
Micromanipulator (MX7600R, Syskiyou, US)
Electrode puller (P-97, Sutter, US)
Glass electrode (BF150-86-10, Sutter, US)
Vibration isolation table and shielding mesh (63-534, TMC, US)
Data acquisition and analysis software (pClamp 10, Axon, US)
Carbon dioxide incubator (HERacell 150i, Thermo, US)
Biological safety cabinet (MODEL 1384, Thermo, US)
Water purifier (Milli Q, Millipore, US)

### 2. Test Method

### 2.1 Cell Culture and Treatment

CHO cells stably expressing hERG were cultured in a cell culture dish with the diameter of 35 mm in a 37° C., 5% CO2 incubator, and passaged at a ratio of 1:5 every 48 h. On the day of the test, the cell culture solution was pipetted, the cells were rinsed once with extracellular fluid, and then a 0.25% Trypsin-EDTA (Invitrogen) solution was added. Digestion was performed at room temperature for 3-5 min. The digestion solution was pipetted, and the cells were resuspended in extracellular fluid and transferred to an experimental dish for electrophysiological recording for later use.

### 2.2 Compound Preparation

On the day of the test, the compound was diluted with DMSO to an intermediate concentration. 10 μL of compound with the intermediate concentration was taken, transferred to 4990 μL of extracellular fluid, and subjected to 500-fold dilution to obtain a final concentration to be tested.

Preparation of positive control compound Cisapride: 10 μL of 150 μM Cisapride DMSO mother liquor was taken, transferred to 4990 μL of extracellular fluid, and subjected to 500-fold dilution to obtain a final concentration 300 nM to be tested.

### 2.3 Electrophysiological Recording Process

CHO (Chinese Hamster Ovary) cells stably expressing hERG potassium channels were taken, and hERG potassium channel currents were recorded at room temperature by means of a whole-cell patch-clamp technique. A glass microelectrode was formed by pulling a glass electrode blank (BF150-86-10, Sutter) with a glass microelectrode puller. The tip resistance was about 2-5 MΩ after perfusion of a pippette solution. The glass microelectrode could be connected to a patch clamp amplifier by inserting the glass microelectrode into an amplifier probe. Clamping voltages and data recording were controlled and recorded by a computer with pClamp 10 software, with a sampling frequency of 10 kHz and a filtering frequency of 2 kHz. After the whole-cell recording was obtained, the cells were clamped at −80 mV, and the step voltage evoking hERG potassium current (I hERG) was changed from −80 mV to +20 mV by giving a 2 s depolarization voltage and then repolarized to −50 mV for 1 s, and returned to −80 mV. Such voltage stimulation was given every 10 s, and the administration process was started after it was determined that the hERG potassium currents were stable (1 min). The compound at each test concentration was given at least 1 min, and at least 2 cells (n≥2) were tested for each concentration.

### 2.4 Data Processing and Analysis

Data analysis was performed by means of pClamp 10 and GraphPad Prism 5.0 software.

The formula for calculating the degree of inhibition of different compound concentrations on hERG potassium currents (the hERG tail current peak value evoked at −50 mV) was:

Inhibition %=[1−(I/Io)]×10000

wherein Inhibition 0% represents the percentage of inhibition of hERG potassium currents by the compound, and I and Io represent the amplitudes of hERG potassium current after administration and before administration, respectively.

Compound IC₅₀ was calculated via the following equation fitting by means of GraphPad Prism 5 software, and the test results were as shown in Table 5:

Y=Bottom+(Top−Bottom)/(1+10\hat{ }((LogIC₅₀−X)*HillSlope))

wherein X is a Log value of a test concentration of a test sample, Y is the inhibition percentage at a corresponding concentration, and Bottom and Top are the minimum inhibition percentage and the maximum inhibition percentage, respectively.

### Test Results

TABLE 5
In-vitro test results of compounds of the present invention

| | 5-HT_{2A} | 5-HT_{2A} inverse | |
|---|---|---|---|
| | antagonistic | agonistic | hERG inhibitory |
| Compound | activity IC₅₀ | activity IC₅₀ | activity IC₅₀ |
| number | (nM) | (nM) | (μM) |
| Pimavanserin | 27.3 | 50 | 0.41 |
| 1 | 8.64 | 22.80 | 0.41 |
| 3 | 11.6 | — | >10 |
| 13 | 1.92 | — | 1.3 |
| 14 | 2.17 | — | — |
| 17 | 1.93 | 3.05 | 1.55 |
| 18 | 5.75 | 1.12 | 0.85 |
| 19 | 6.94 | 49.85 | 0.48 |
| 20 | 9.14 | — | 0.68 |
| 21 | 2.1 | 34.15 | 0.91 |
| 23 | 6.02 | 5.64 | >10 |
| 24 | 3.49 | 2.54 | 3.61 |
| 25 | 6.73 | 16.87 | 1.03 |
| 26 | 6.86 | 9.79 | 0.53 |
| 30 | 9.92 | 10.66 | — |
| 31 | 17.4 | 30.04 | 1.85 |
| 37 | 2.05 | 42.78 | 3.99 |
| 38 | — | 10.04 | — |
| 41 | 9.98 | 4.29 | 0.79 |
| 45 | 2.42 | 7.04 | 5.5 |
| 46 | 4.15 | 11.06 | 10.4 |
| 47 | 3.13 | 9.22 | 1.39 |
| 48 | 3.14 | 22.5 | 2.4 |
| 49 | 4.1 | 7.3 | 2.43 |
| 50 | 20.9 | — | 0.55 |
| 52 | — | 42.74 | — |
| 55 | 4.7 | 2.23 | 0.69 |
| 56 | 5.77 | 9.90 | — |
| 57 | 6.41 | 6.83 | — |
| 59 | 0.54 | 2.04 | 0.81 |
| 60 | 2.11 | 16.4 | >10 |
| 61 | 3.32 | 31.21 | 2.55 |
| 62 | 2.35 | 32.8 | 1.43 |
| 63 | — | 20.84 | — |
| 65 | 7.98 | 18.4 | 0.67 |
| 66 | 12 | 8.85 | — |
| 70 | 8.09 | 12.33 | 0.5 |
| 71 | 9.52 | — | 3.32 |
| 74 | 8.95 | — | — |
| 75 | 8.35 | 14.37 | — |
| 76 | 20.68 | — | — |
| 77 | 6.59 | 6.44 | — |
| 78 | 2.17 | 6.01 | 1.74 |
| 81 | 2.78 | 30.06 | 0.68 |
| 82 | 3.78 | 1.95 | 0.5 |
| 89 | 7.72 | — | 2.05 |
| 91 | 14.7 | — | 0.58 |
| 92 | 4.64 | 6.54 | — |
| 93 | 4.26 | 10.58 | — |
| 94 | 4.29 | 7.59 | — |
| 95 | 5.13 | 27.92 | — |
| 96 | 11.39 | 26.38 | — |
| 98 | 21.25 | — | 1.46 |
| 99 | 17.27 | 33.34 | 0.65 |

The results show that:
the compounds of the present invention have superior 5-HT_{2A} antagonistic activity and/or 5-HT_{2A} inverse agonistic activity to those of pimavanserin, and have lower cardiotoxicity.

### Test Example 3. In-Vitro Stability Evaluation of Pimavanserin and Compound 59 in Liver Microsomes

### 1 Solution Formulation

1) formulation of test sample working solution: the test sample was diluted to 100 μM with methanol;
2) formulation of liver microsome working solution: liver microsomes were diluted to 0.56 mg/ml with a 100 mM phosphate buffer;
3) formulation of reduced nicotinamide adenine dinucleotide phosphate (NADPH) working solution: an appropriate amount of NADPH was weighted and diluted to 20 mM with a phosphate buffer, and then a 60 mM MgCl₂ solution with the equal volume was added;
4) formulation of stop solution: tolbutamide was diluted to 20 ng/mL with acetonitrile to serve as a stop solution containing an internal standard.

### 2 Incubation Process

1) anti-adsorption EP tubes for incubation were prepared, and species, test samples, reference substances (testosterone and dextromethorphan), time points (0 min, 5 min, 10 min, 20 min, 30 min, 60 min, Blank60 and NCF60), etc. were labeled;
2) 2 μL of test sample or reference substance working solution and 178 μL of liver microsome working solution were added to each tube, 2 μL of acetonitrile in place of the test samples was added to the Blank tube, and the mixture was placed in a 37° C. water bath kettle and pre-incubated for about 10 min, wherein each sample was divided in triplicate aliquots;
3) after the pre-incubation was completed, 20 μL of NADPH working solution was added to each tube except for 0 min and NCF60 to initiate the reaction, and 20 μL of phosphate buffer (containing 30 mM MgCl₂) was added to the NCF60 tube, wherein in the incubation system, the test sample or the reference substance had a final concentration of 1 μM, the liver microsomes had a final concentration of 0.5 mg/mL, the NADPH had a final concentration of 1 mM, and the MgCl₂ had a final concentration of 3 mM;
4) 600 μL of stop solution was added to the 0-min sample followed by an NADPH working solution, and 600 μL of stop solution was added to stop the reaction after each sample was incubated for a corresponding period of time;
5) each sample was vortexed for 30 s after the reaction was stopped and then centrifuged at 13500 rpm for 10 m; 100 L of supernatant was added to an EP tube, 100 μL of Milli-Q water was added, and the resulting mixture was vortexed and mixed uniformly; and LC-MS/MS analysis was performed by using the method in Table 6; and
6) testosterone and dextromethorphan were used as positive controls under the same conditions to test the stability and reliability of the system.

TABLE 6
LC-MS-MS analysis methods

| | |
|---|---|
| Analyte | Pimavanserin, compound 59, |
| | testosterone and dextromethorphan |

Liquid-phase method

| Mobile phase A | 0.1% formic acid in water | | | |
|---|---|---|---|---|
| Mobile phase B | 0.1% formic acid in methanol | | | |
| Chromatographic | ACQUITY UPLC BEH C18 2.1 × 50 mm, | | | |
| column | particle size 1.7 uM Waters | | | |
| Internal standard | Tolbutamide | | | |
| Injection volume | 5 μL | | | |
| Gradient | Time | Flow rate | A | B |
| | (min) | (μL/min) | (%) | (%) |
| | 0.00 | 0.5 | 80 | 20 |
| | 1.00 | 0.5 | 80 | 20 |
| | 1.10 | 0.5 | 10 | 90 |
| | 2.00 | 0.5 | 10 | 90 |
| | 2.30 | 0.5 | 80 | 20 |
| | 3.00 | 0.5 | 80 | 20 |

Mass spectrometry method

| Scanning mode | MRM | | | | |
|---|---|---|---|---|---|
| Compound name | Ion pair | DP | EP | CE | CXP |
| | | (eV) | (eV) | (eV) | (eV) |
| Pimavanserin | 428.2/223.2 | 84 | 11 | 24 | 18 |
| Compound 59 | 429.2/224.2 | 50 | 6 | 23 | 8 |
| Testosterone | 289.1/97.0 | 99 | 14 | 29 | 8 |
| Dextromethorphan | 272.2/171.1 | 130 | 10 | 46 | 7 |
| Tolbutamide | 271.1/155.3 | 30 | 8 | 21 | 7 |

### 3 Data Analysis

The remaining percentage of the test sample was tested 60 min later. The test results were as shown in Table 7.

TABLE 7
Test data of compounds of the present invention
in human and rat liver microsomes

| | | Remaining amount |
|---|---|---|
| Compounds | Species | 60 min later % |
| Pimavanserin | Dog | 29.72 |
| | Human | 77.44 |
| Compound 59 | Dog | 76.37 |
| | Human | 89.19 |

The results show that: compound 59 has obviously superior stability to that of pimavanserin in dog and human liver microsomes in vitro, and has better druggability.

### Test Example 4. In-Vivo Pharmacokinetic Test of Pimavanserin and Compound 59 in Rats

### 1 Test Drugs

Pimavanserin: Purchased from MCE Corporation.
Compound 59: Prepared according to the previous examples

### 2 Test Method

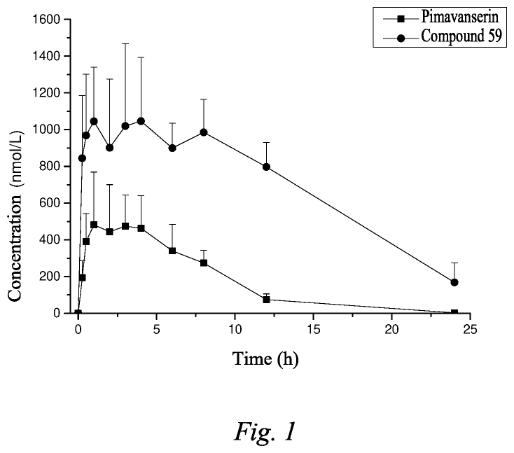

Eight SD rats weighting about 220 g were randomly divided into 2 groups, 4 rats per group, fasted for 12 h before administration, and intragastrically given pimavanserin and compound 59 at a dose of 46.7 μmol/kg, respectively, with vehicles both being 20% Solutol. Blood was collected before administration and 0.25 h, 0.5 h, 1 h, 2 h, 3 h, 4 h, 6 h, 8 h, 12 h and 24 h after administration, respectively and placed in a heparinized EP tube, and the plasma was centrifugally separated. After the plasma was pre-treated, the concentrations of compounds in the plasma were determined by LC-MS/MS, and the pharmacokinetic parameters were calculated. The test results were as shown in FIG. 1 and Table 8.

TABLE 8
Comparison of pharmacokinetic parameters of pimavanserin
and compound 59 after intragastric administration

| Parameter | Unit | Pimavanserin | Compound 59 |
|---|---|---|---|
| Tₘₐₓ | h | 2.75 ± 1.26 | 5.88 ± 5.14 |
| Cₘₐₓ | nmol/L | 536 ± 260 | 1135 ± 365 |
| AUCₗₐₛₜ | nmol/L * h | 4286 ± 1664 | 16987 ± 2969 |
| T_{1/2} | h | 2.38 ± 0.8 | 5.51 ± 1.33 |
| MRTₗₐₛₜ | h | 5.61 ± 0.34 | 8.66 ± 0.89 |

The results show that: the T_{1/2} of compound 59 is higher than that of pimavanserin; the Cₘₐₓ of compound 59 is 2 times that of pimavanserin; the AUCₗₐₛₜ of compound 59 is 4 times that of pimavanserin; and the in-vivo pharmacokinetic properties of compound 59 are obviously better than those of pimavanserin.

1. A compound of formula (I) or a pharmaceutically acceptable salt thereof, 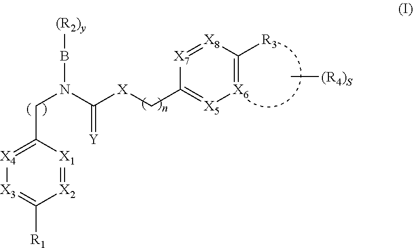
wherein
at least one of X₁ and X₄ is N, and the other one is optionally CR₁ or N;
X₂ and X₃ are each independently selected from CR₁ and N;
X₅ is independently selected from CR₃ₐ or N;
X₆ is independently selected from CR_{3b} or N;
X₇ is independently selected from CR_{3c} or N;
X₈ is independently selected from CR_{3d} or N;
group B is linear or branched C₁₋₆ alkyl or a 5-6-membered nitrogen heterocyclic group, and the linear or branched C₁₋₆ alkyl or the 5-6-membered nitrogen heterocyclic group is optionally substituted with one or more deuterium atoms;
each R₁ is the same or different and is independently selected from a hydrogen atom, linear or branched C₁₋₁₀ alkyl or halogen;
each R₂ is the same or different and is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3b}, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or when X₆ is CR_{3b}, X₆ and R₃ together with the atoms to which they are attached form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy;
X is selected from —NH— or —(CH₂)₁₋₄NH—;
Y is selected from O or S;
m and n are independently selected from 0, 1, 2 and 3;
s is independently selected from 1, 2, 3, 4, 5 and 6; and
y is independently selected from 0, 1, 2, 3, 4 and 5.

2. The compound of claim 1, wherein the compound is represented by formula (II) 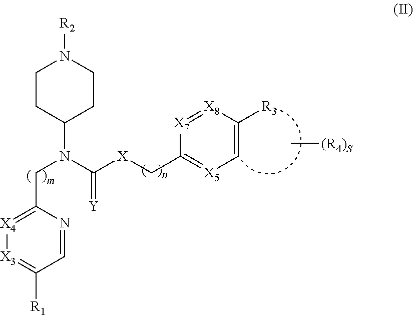
and wherein,
X₃ and X₄ are each independently selected from CR₁ and N;
X₅ is independently selected from CR₃ₐ or N;
X₇ is independently selected from CR_{3c} or N;
X₈ is independently selected from CR_{3d} or N;
each R₁ is the same or different and is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, pyrroline or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy;
X is selected from —NH— or —(CH₂)₁₋₄NH—;
Y is selected from O or S;
m and n are independently selected from 0, 1, 2 and 3; and
s is independently selected from 1, 2, 3, 4, 5 and 6.

3. The compound of claim 1, wherein the compound is represented by formula (III) 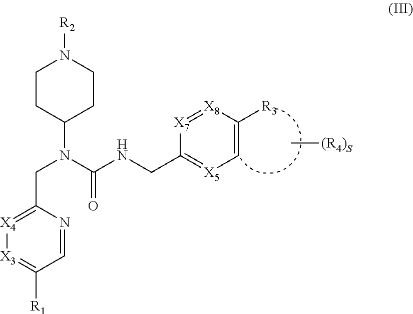
and wherein,
X₃ and X₄ are each independently selected from CR₁ and N;
X₅ is independently selected from CR₃ₐ or N;
X₇ is independently selected from CR_{3c} or N;
X₈ is independently selected from CR_{3d} or N;
each R₁ is the same or different and is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R₃ₐ, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy; and
s is independently selected from 1, 2, 3, 4, 5 and 6.

4. The compound of claim 1, wherein the compound is represented by formula (IV) 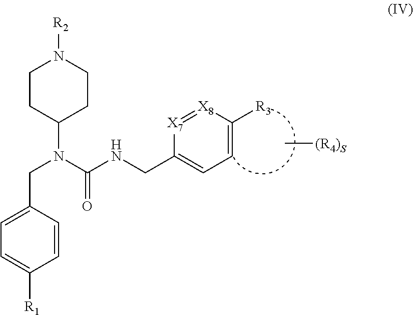
and wherein,
X₇ is independently selected from CR_{3c} or N;
X₈ is independently selected from CR_{3d} or N;
R₁ is independently selected from a hydrogen atom, linear or branched C₁₋₁₀ alkyl or halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms;
R₃, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy;
or R₃ and the carbon atom to which it is attached together with adjacent carbon atoms form a ring system which is selected from dihydrofuran, dihydropyrrole or dihydrothiophene and are substituted with one or more of the same or different R₄, and each R₄ is independently selected from a hydrogen atom, halogen, linear or branched C₁₋₁₀ alkyl, linear or branched C₁₋₁₀ alkoxy or linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, hydroxyl and linear or branched C₁₋₁₀ alkoxy; and
s is independently selected from 1, 2, 3, 4, 5 and 6.

5. The compound of claim 1, wherein the compound is represented by formula (V) 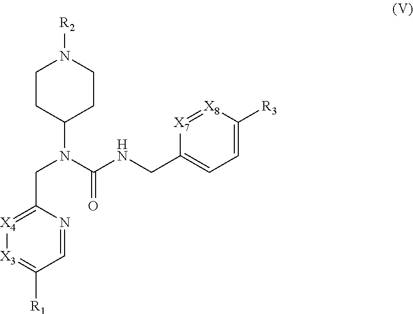
and wherein,
X₃ is independently selected from CR₅ or N;
X₄ is independently selected from CR₆ or N;
X₇ is independently selected from CR_{3c} or N;
X₈ is independently selected from CR_{3d} or N;
R₁, R₅ and R₆ are the same or different and are each independently selected from a hydrogen atom, linear or branched C₁₋₁₀ alkyl and halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms; and
R₃, R_{3c} and R_{3d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy.

6. The compound of claim 1, wherein the compound is represented by formula (VI) 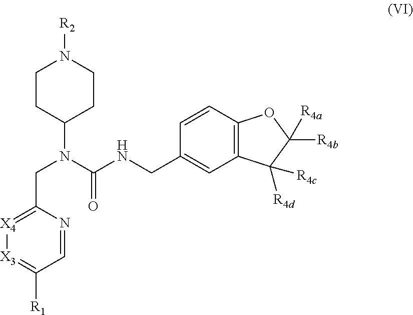
and wherein,
X₃ is independently selected from CR₅ or N;
X₄ is independently selected from CR₆ or N;
R₁, R₅ and R₆ are the same or different and are each independently selected from a hydrogen atom, linear or branched C₁₋₁₀ alkyl and halogen;
R₂ is independently selected from a hydrogen atom, a deuterium atom, linear or branched C₁₋₁₀ alkyl, (linear or branched C₁₋₆ alkyl)₂ amine or 3-8-membered cycloalkyl, and the linear or branched C₁₋₁₀ alkyl, the (linear or branched C₁₋₆ alkyl)₂ amine or the 3-8-membered cycloalkyl is optionally substituted with one or more deuterium atoms; and
R₄ₐ, R_{4b} and R_{4c} and R_{4d} are the same or different and are each independently selected from a hydrogen atom, halogen, hydroxyl, linear or branched C₁₋₁₀ alkyl, linear or branched C₁₋₁₀ alkoxy and linear or branched C₁₋₁₀ haloalkoxy, wherein the linear or branched C₁₋₁₀ alkyl and the linear or branched C₁₋₁₀ alkoxy are substituted with one or more substituents selected from a hydrogen atom, halogen, hydroxyl and linear or branched C₁₋₁₀ alkoxy.

7. The compound or the pharmaceutically acceptable salt thereof according to claim 1, wherein
R₁, R₅ and R₆ are the same or different and are each independently selected from a hydrogen atom, F, Cl, Br, I, methyl, ethyl, propyl, isopropyl, butyl, isobutyl, sec-butyl and tert-butyl;
group B is —CH₂—, —(CH₂)₂—, —(CH₂)₃—, —(CH₂)₄—, -CD₂-, -(CD₂)₂-, -(CD₂)₃- or -(CD₂)₄-, and R₂ is independently selected from dimethylamine or diethylamine, wherein the dimethylamine or the diethylamine is optionally substituted with one or more deuterium atoms;
or group B is selected from piperidinyl, wherein the piperidinyl is optionally substituted with one or more deuterium atoms, and R₂ is independently selected from a hydrogen atom, a deuterium atom, methyl, ethyl, propyl, isopropyl, butyl, isobutyl, sec-butyl, tert-butyl, cyclopropyl, cyclobutyl, cyclopentyl or cyclohexyl, wherein the methyl, the ethyl, the propyl, the isopropyl, the butyl, the isobutyl, the sec-butyl, the tert-butyl, the cyclopropyl, the cyclobutyl, the cyclopentyl or the cyclohexyl is optionally substituted with one or more deuterium atoms;
R₃, R₃ₐ, R_{3b}, R_{3c} and R_{3d} are the same or different and are each independently selected from a hydrogen atom, F, Cl, Br, I, hydroxyl, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, oxy, ethoxy, propoxy, isopropoxy, butoxy, isobutoxy, sec-butoxy, tert-butoxy, pentyloxy, hexyloxy, fluoromethoxy, difluoromethoxy, trichloromethoxy, trifluoromethoxy, 2-fluoroethoxy, 2,2-difluoroethoxy, 2,2,2-trifluoroethoxy, tetrafluoroethoxy, pentafluoroethoxy, 3-fluoropropoxy, 3,3-difluoropropoxy, 2,2′-difluoroisopropoxy, 3,3,3-trifluoropropoxy, 4-fluorobutoxy, 4,4-difluorobutoxy, 4,4,4-trifluorobutoxy, 2-fluoro-2-methylpropyl, 5,5,5-trifluoropentyloxy, 6,6,6-trifluorohexyloxy, 2-methyl-3-hydroxy-butyl and i-Pr—O—CH₂—; and
R₄ and/or R₄ₐ, R_{4b}, R_{4c} and R_{4d} are the same or different and are each independently selected from a hydrogen atom, methyl, ethyl, propyl, isopropyl, butyl, isobutyl, sec-butyl and tert-butyl.

8. A compound or a pharmaceutically acceptable salt thereof or a deuterated analog thereof, which is selected from the following:

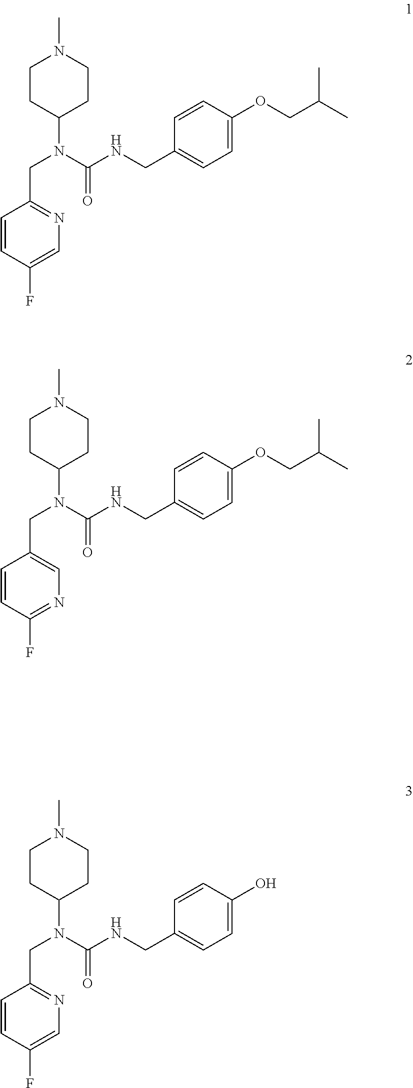 [SMILES: CC(C)COc1ccc(CNC(=O)N(Cc2ccc(F)cn2)C2CCN(C)CC2)cc1 ; CC(C)COc1ccc(CNC(=O)N(Cc2ccc(F)nc2)C2CCN(C)CC2)cc1 ; CN1CCC(N(Cc2ccc(F)cn2)C(=O)NCc2ccc(O)cc2)CC1]

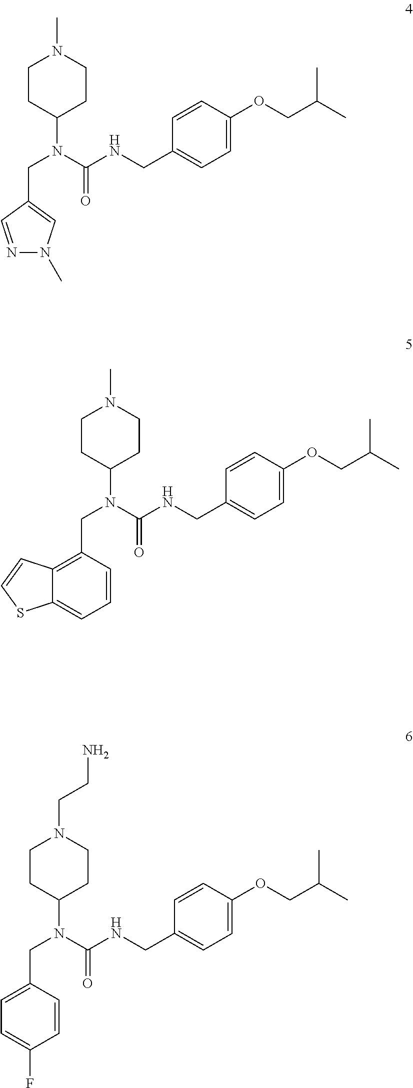 [SMILES: CC(C)COc1ccc(CNC(=O)N(Cc2cnn(C)c2)C2CCN(C)CC2)cc1 ; CC(C)COc1ccc(CNC(=O)N(Cc2cccc3sccc23)C2CCN(C)CC2)cc1 ; CC(C)COc1ccc(CNC(=O)N(Cc2ccc(F)cc2)C2CCN(CCN)CC2)cc1]

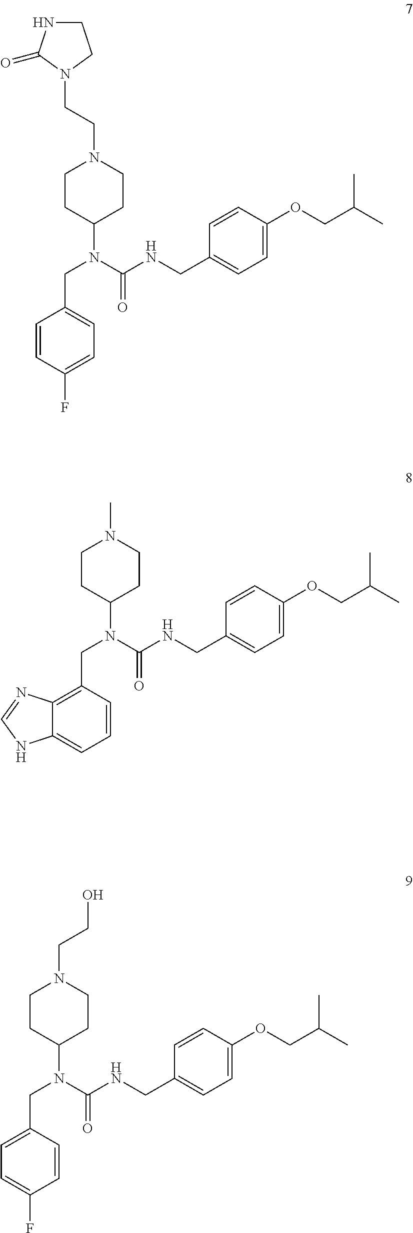 [SMILES: CC(C)COc1ccc(CNC(=O)N(Cc2ccc(F)cc2)C2CCN(CCN3CCNC3=O)CC2)cc1 ; CC(C)COc1ccc(CNC(=O)N(Cc2cccc3[nH]cnc23)C2CCN(C)CC2)cc1 ; CC(C)COc1ccc(CNC(=O)N(Cc2ccc(F)cc2)C2CCN(CCO)CC2)cc1]

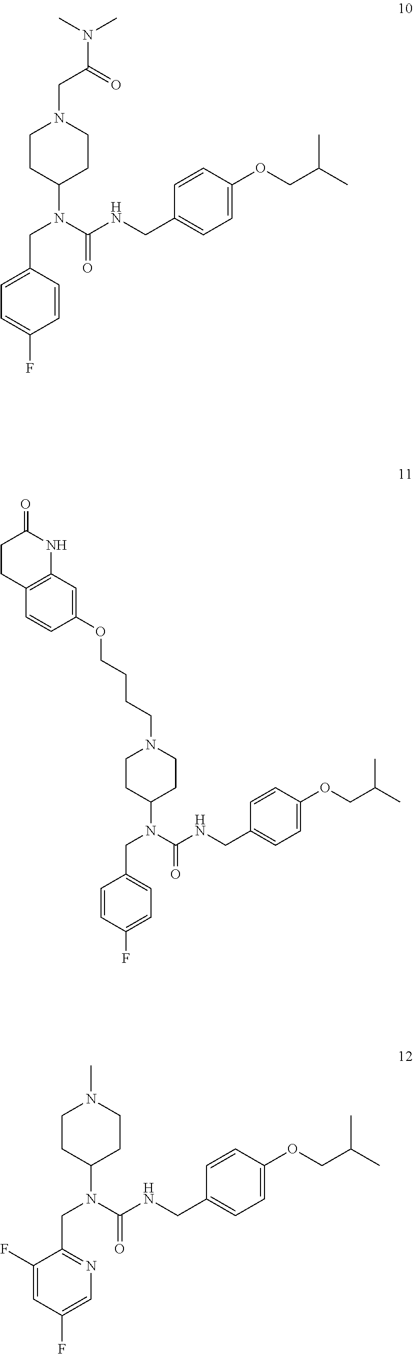 [SMILES: CC(C)COc1ccc(CNC(=O)N(Cc2ccc(F)cc2)C2CCN(CC(=O)N(C)C)CC2)cc1 ; CC(C)COc1ccc(CNC(=O)N(Cc2ccc(F)cc2)C2CCN(CCCCOc3ccc4c(c3)NC(=O)CC4)CC2)cc1 ; CC(C)COc1ccc(CNC(=O)N(Cc2ncc(F)cc2F)C2CCN(C)CC2)cc1]

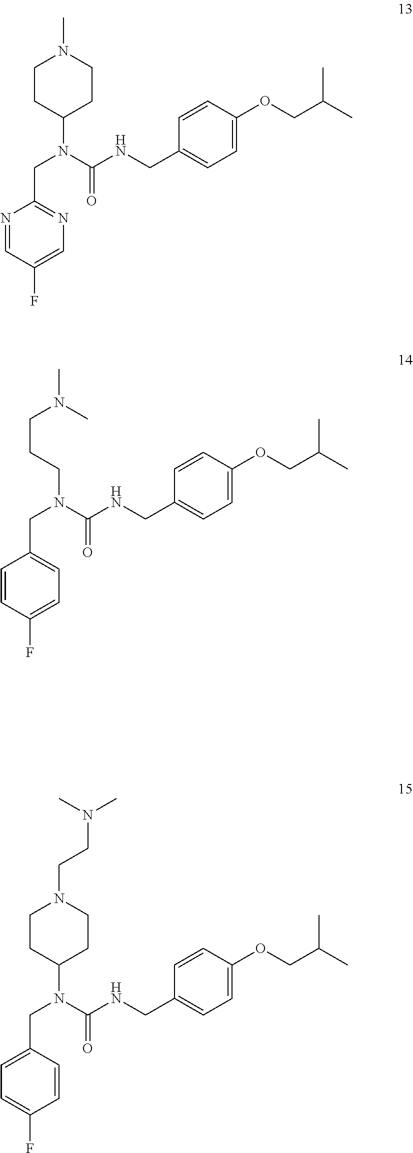 [SMILES: CC(C)COc1ccc(CNC(=O)N(Cc2ncc(F)cn2)C2CCN(C)CC2)cc1 ; CC(C)COc1ccc(CNC(=O)N(CCCN(C)C)Cc2ccc(F)cc2)cc1 ; CC(C)COc1ccc(CNC(=O)N(Cc2ccc(F)cc2)C2CCN(CCN(C)C)CC2)cc1]

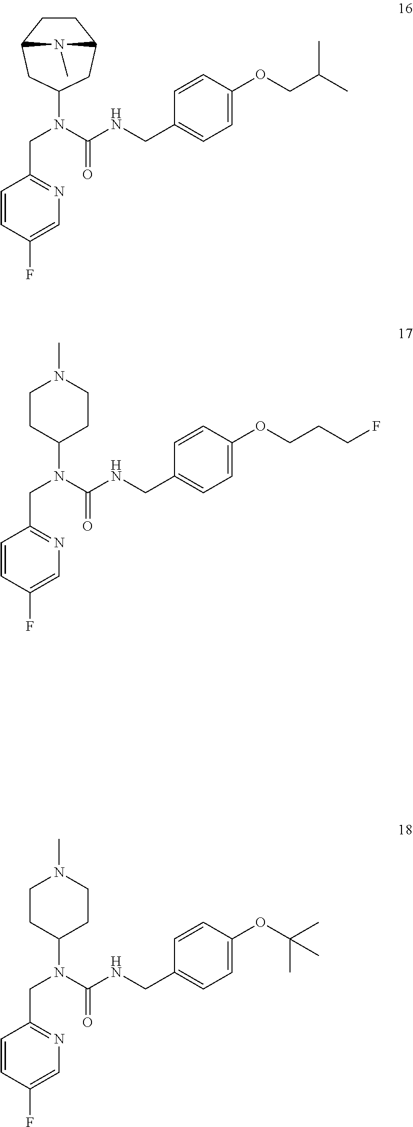

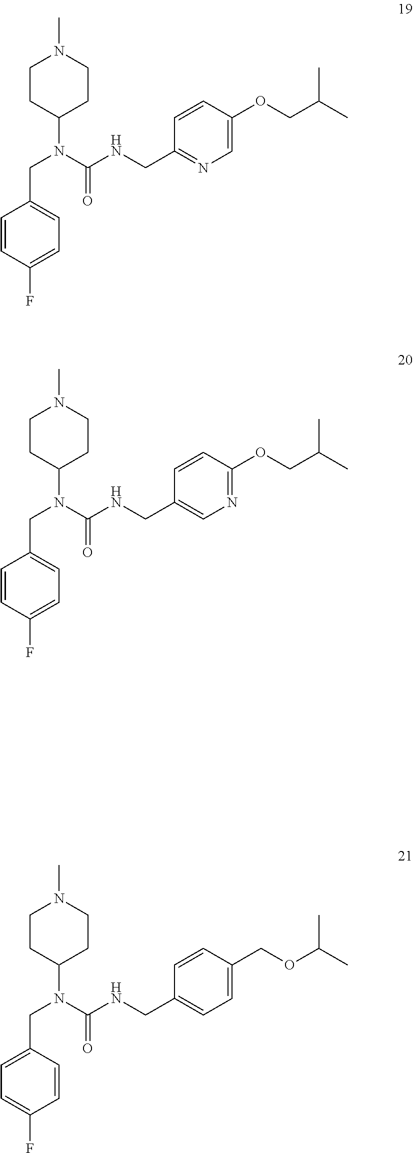 [SMILES: CC(C)COc1ccc(CNC(=O)N(Cc2ccc(F)cc2)C2CCN(C)CC2)nc1 ; CC(C)COc1ccc(CNC(=O)N(Cc2ccc(F)cc2)C2CCN(C)CC2)cn1 ; CC(C)OCc1ccc(CNC(=O)N(Cc2ccc(F)cc2)C2CCN(C)CC2)cc1]

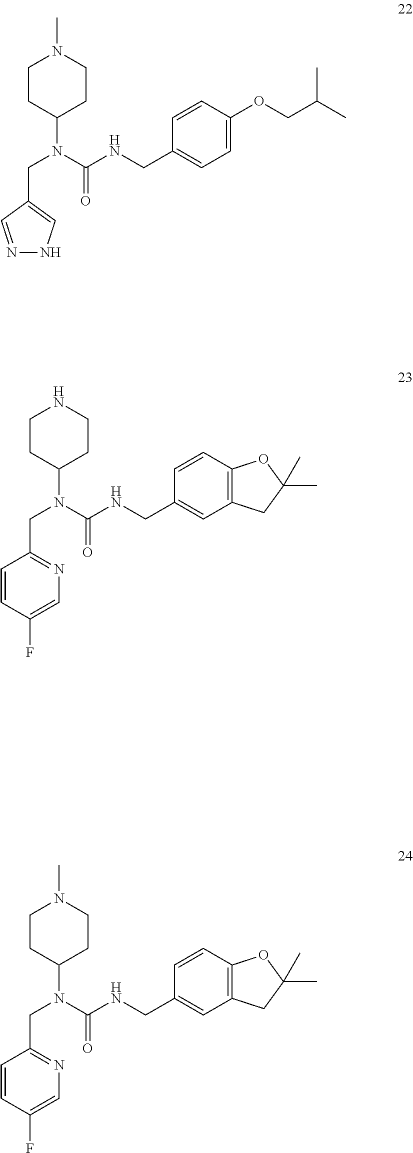 [SMILES: CC(C)COc1ccc(CNC(=O)N(Cc2cn[nH]c2)C2CCN(C)CC2)cc1 ; CC1(C)Cc2cc(CNC(=O)N(Cc3ccc(F)cn3)C3CCNCC3)ccc2O1 ; CN1CCC(N(Cc2ccc(F)cn2)C(=O)NCc2ccc3c(c2)CC(C)(C)O3)CC1]

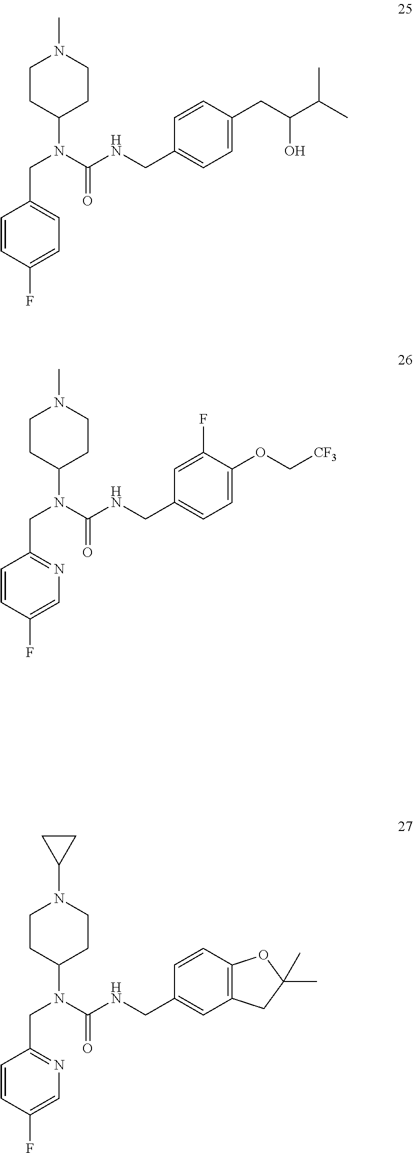 [SMILES: CC(C)C(O)Cc1ccc(CNC(=O)N(Cc2ccc(F)cc2)C2CCN(C)CC2)cc1 ; CN1CCC(N(Cc2ccc(F)cn2)C(=O)NCc2ccc(OCC(F)(F)F)c(F)c2)CC1 ; CC1(C)Cc2cc(CNC(=O)N(Cc3ccc(F)cn3)C3CCN(C4CC4)CC3)ccc2O1]

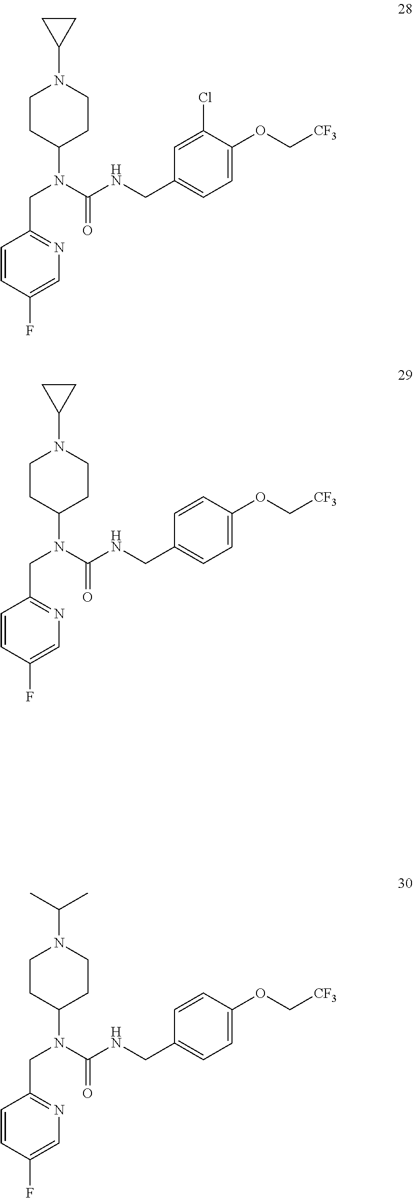 [SMILES: O=C(NCc1ccc(OCC(F)(F)F)c(Cl)c1)N(Cc1ccc(F)cn1)C1CCN(C2CC2)CC1 ; O=C(NCc1ccc(OCC(F)(F)F)cc1)N(Cc1ccc(F)cn1)C1CCN(C2CC2)CC1 ; CC(C)N1CCC(N(Cc2ccc(F)cn2)C(=O)NCc2ccc(OCC(F)(F)F)cc2)CC1]

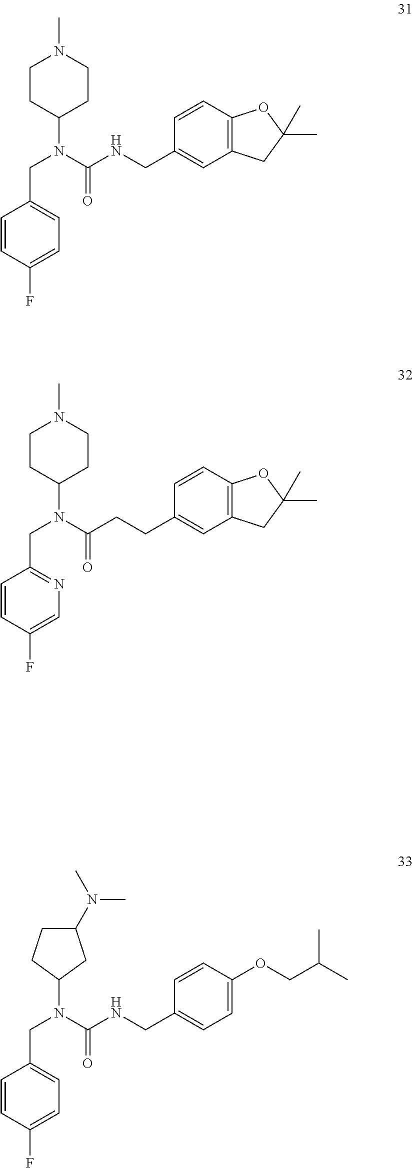 [SMILES: CN1CCC(N(Cc2ccc(F)cc2)C(=O)NCc2ccc3c(c2)CC(C)(C)O3)CC1 ; CN1CCC(N(Cc2ccc(F)cn2)C(=O)CCc2ccc3c(c2)CC(C)(C)O3)CC1 ; CC(C)COc1ccc(CNC(=O)N(Cc2ccc(F)cc2)C2CCC(N(C)C)C2)cc1]

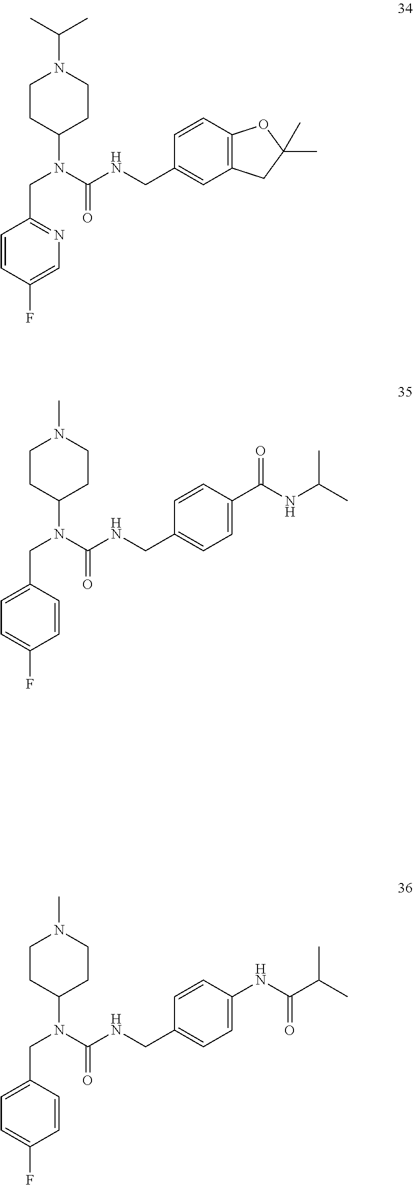 [SMILES: CC(C)N1CCC(N(Cc2ccc(F)cn2)C(=O)NCc2ccc3c(c2)CC(C)(C)O3)CC1 ; CC(C)NC(=O)c1ccc(CNC(=O)N(Cc2ccc(F)cc2)C2CCN(C)CC2)cc1 ; CC(C)C(=O)Nc1ccc(CNC(=O)N(Cc2ccc(F)cc2)C2CCN(C)CC2)cc1]

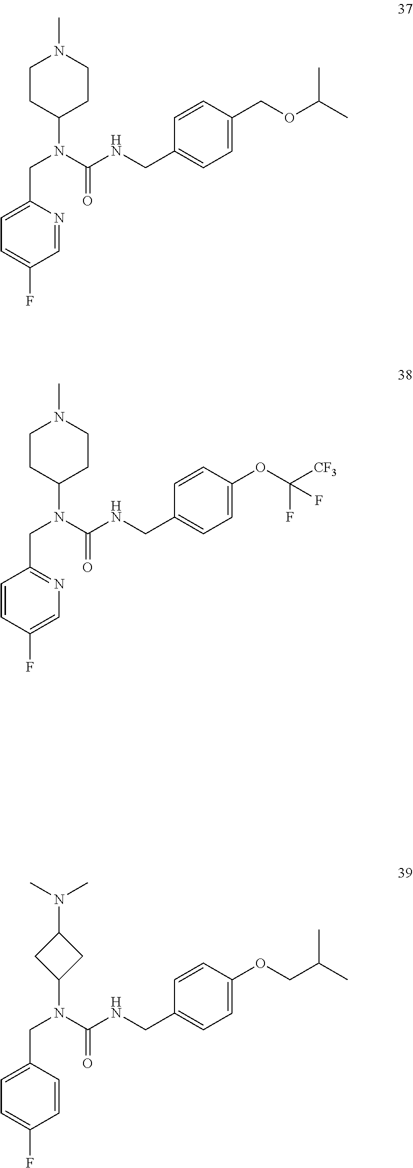 [SMILES: CC(C)OCc1ccc(CNC(=O)N(Cc2ccc(F)cn2)C2CCN(C)CC2)cc1 ; CN1CCC(N(Cc2ccc(F)cn2)C(=O)NCc2ccc(OC(F)(F)C(F)(F)F)cc2)CC1 ; CC(C)COc1ccc(CNC(=O)N(Cc2ccc(F)cc2)C2CC(N(C)C)C2)cc1]

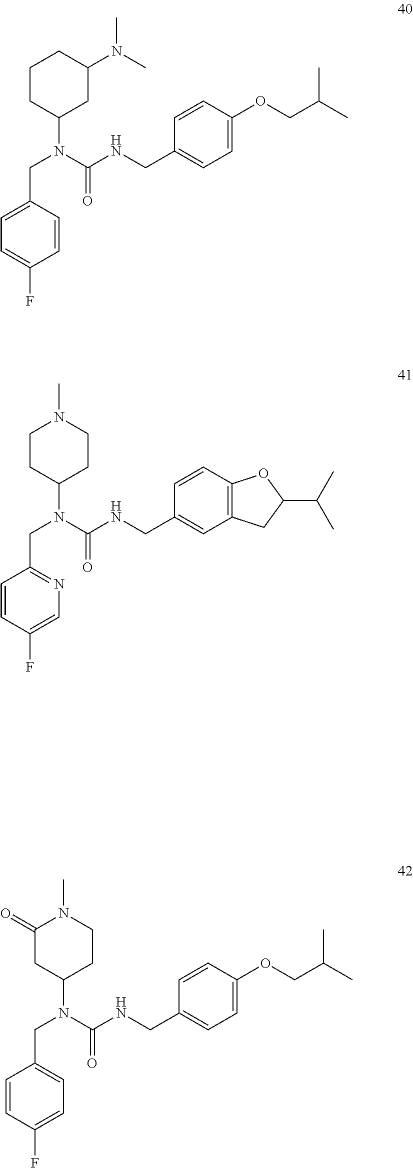 [SMILES: CC(C)COc1ccc(CNC(=O)N(Cc2ccc(F)cc2)C2CCCC(N(C)C)C2)cc1 ; CC(C)C1Cc2cc(CNC(=O)N(Cc3ccc(F)cn3)C3CCN(C)CC3)ccc2O1 ; CC(C)COc1ccc(CNC(=O)N(Cc2ccc(F)cc2)C2CCN(C)C(=O)C2)cc1]

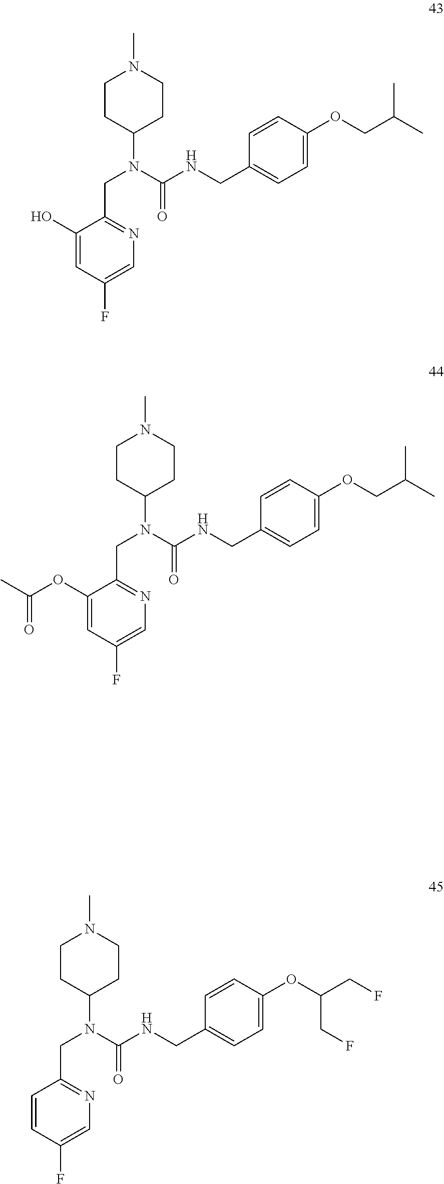

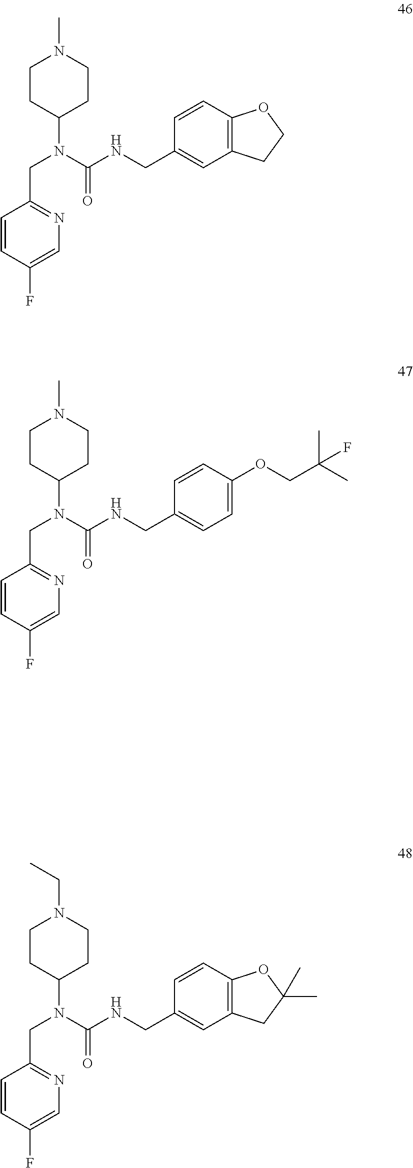 [SMILES: CN1CCC(N(Cc2ccc(F)cn2)C(=O)NCc2ccc3c(c2)CCO3)CC1 ; CN1CCC(N(Cc2ccc(F)cn2)C(=O)NCc2ccc(OCC(C)(C)F)cc2)CC1 ; CCN1CCC(N(Cc2ccc(F)cn2)C(=O)NCc2ccc3c(c2)CC(C)(C)O3)CC1]

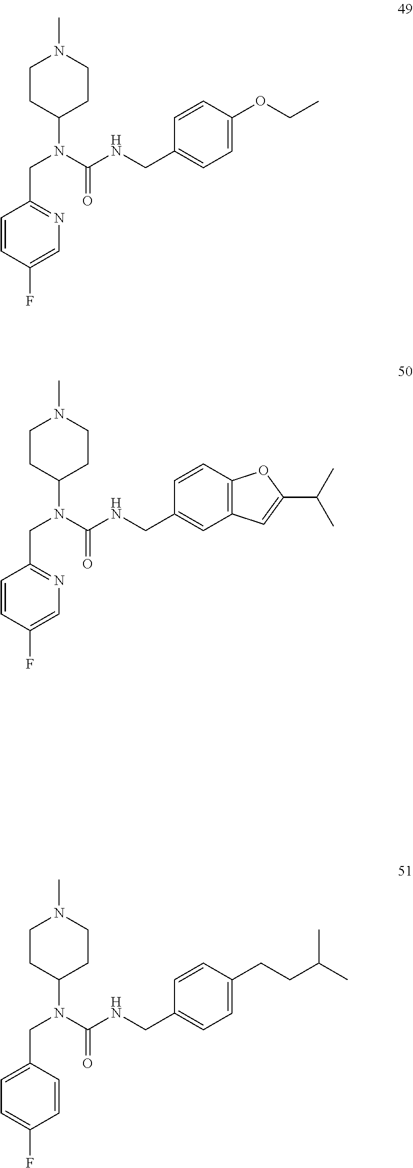 [SMILES: CCOc1ccc(CNC(=O)N(Cc2ccc(F)cn2)C2CCN(C)CC2)cc1 ; CC(C)c1cc2cc(CNC(=O)N(Cc3ccc(F)cn3)C3CCN(C)CC3)ccc2o1 ; CC(C)CCc1ccc(CNC(=O)N(Cc2ccc(F)cc2)C2CCN(C)CC2)cc1]

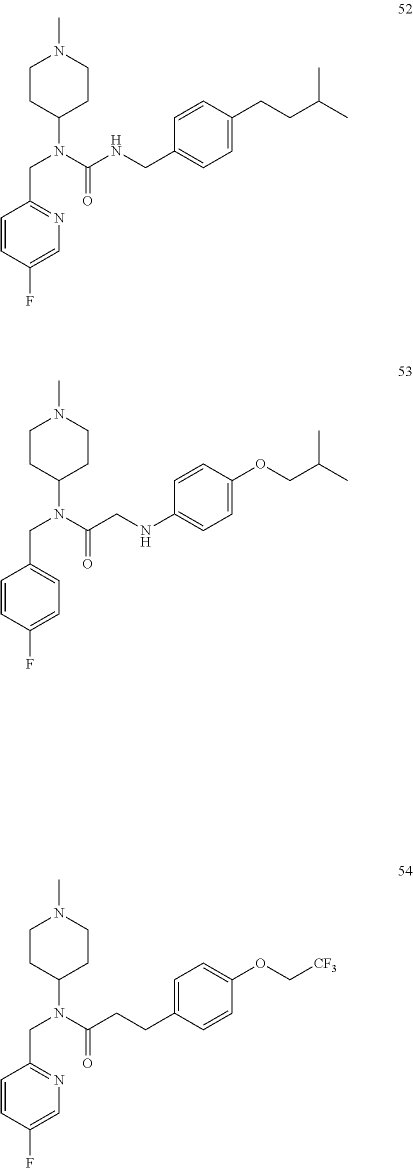 [SMILES: CC(C)CCc1ccc(CNC(=O)N(Cc2ccc(F)cn2)C2CCN(C)CC2)cc1 ; CC(C)COc1ccc(NCC(=O)N(Cc2ccc(F)cc2)C2CCN(C)CC2)cc1 ; CN1CCC(N(Cc2ccc(F)cn2)C(=O)CCc2ccc(OCC(F)(F)F)cc2)CC1]

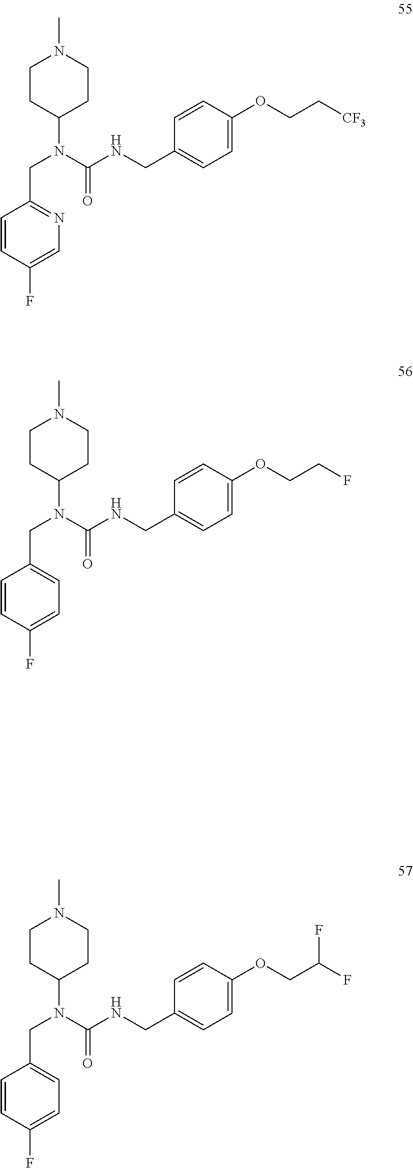 [SMILES: CN1CCC(N(Cc2ccc(F)cn2)C(=O)NCc2ccc(OCCC(F)(F)F)cc2)CC1 ; CN1CCC(N(Cc2ccc(F)cc2)C(=O)NCc2ccc(OCCF)cc2)CC1 ; CN1CCC(N(Cc2ccc(F)cc2)C(=O)NCc2ccc(OCC(F)F)cc2)CC1]

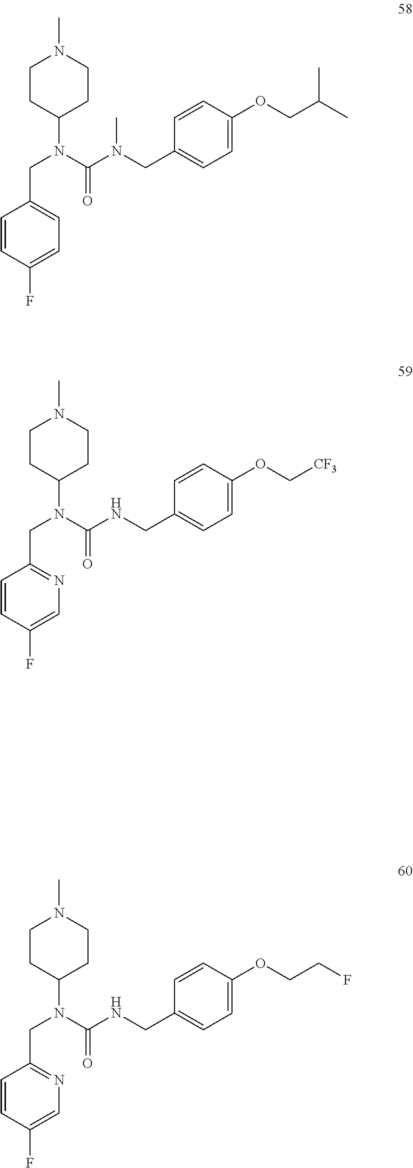 [SMILES: CC(C)COc1ccc(CN(C)C(=O)N(Cc2ccc(F)cc2)C2CCN(C)CC2)cc1 ; CN1CCC(N(Cc2ccc(F)cn2)C(=O)NCc2ccc(OCC(F)(F)F)cc2)CC1 ; CN1CCC(N(Cc2ccc(F)cn2)C(=O)NCc2ccc(OCCF)cc2)CC1]

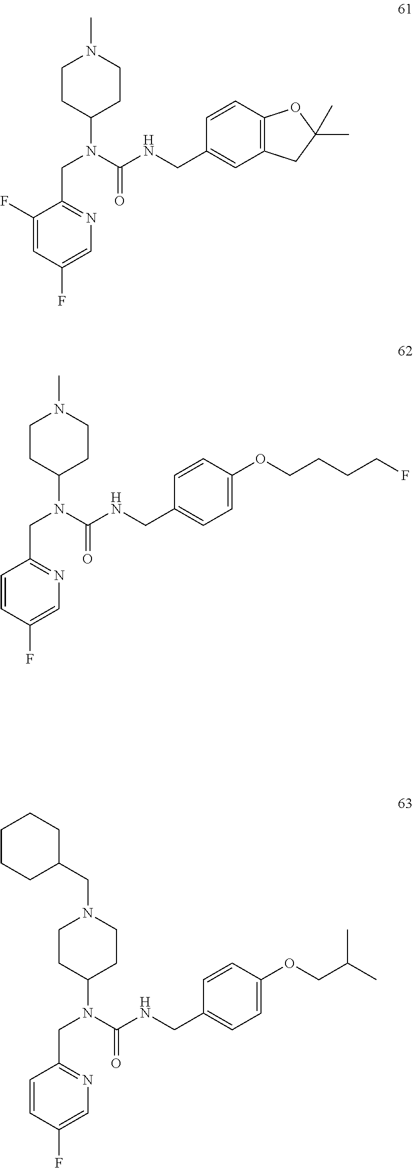 [SMILES: CN1CCC(N(Cc2ncc(F)cc2F)C(=O)NCc2ccc3c(c2)CC(C)(C)O3)CC1 ; CN1CCC(N(Cc2ccc(F)cn2)C(=O)NCc2ccc(OCCCCF)cc2)CC1 ; CC(C)COc1ccc(CNC(=O)N(Cc2ccc(F)cn2)C2CCN(CC3CCCCC3)CC2)cc1]

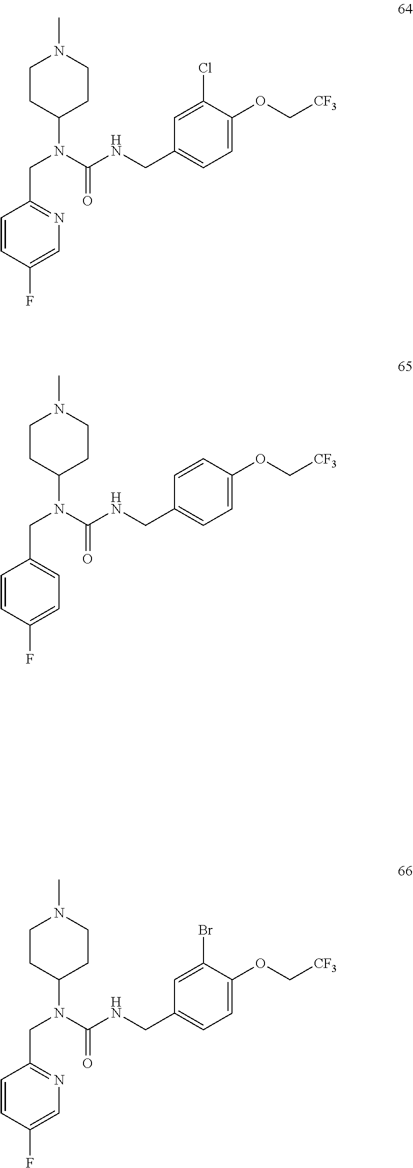 [SMILES: CN1CCC(N(Cc2ccc(F)cn2)C(=O)NCc2ccc(OCC(F)(F)F)c(Cl)c2)CC1 ; CN1CCC(N(Cc2ccc(F)cc2)C(=O)NCc2ccc(OCC(F)(F)F)cc2)CC1 ; CN1CCC(N(Cc2ccc(F)cn2)C(=O)NCc2ccc(OCC(F)(F)F)c(Br)c2)CC1]

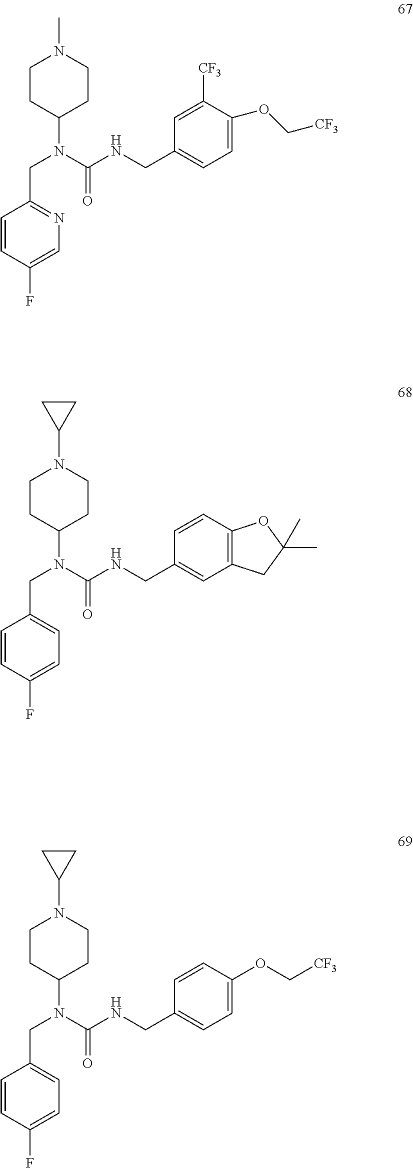 [SMILES: CN1CCC(N(Cc2ccc(F)cn2)C(=O)NCc2ccc(OCC(F)(F)F)c(C(F)(F)F)c2)CC1 ; CC1(C)Cc2cc(CNC(=O)N(Cc3ccc(F)cc3)C3CCN(C4CC4)CC3)ccc2O1 ; O=C(NCc1ccc(OCC(F)(F)F)cc1)N(Cc1ccc(F)cc1)C1CCN(C2CC2)CC1]

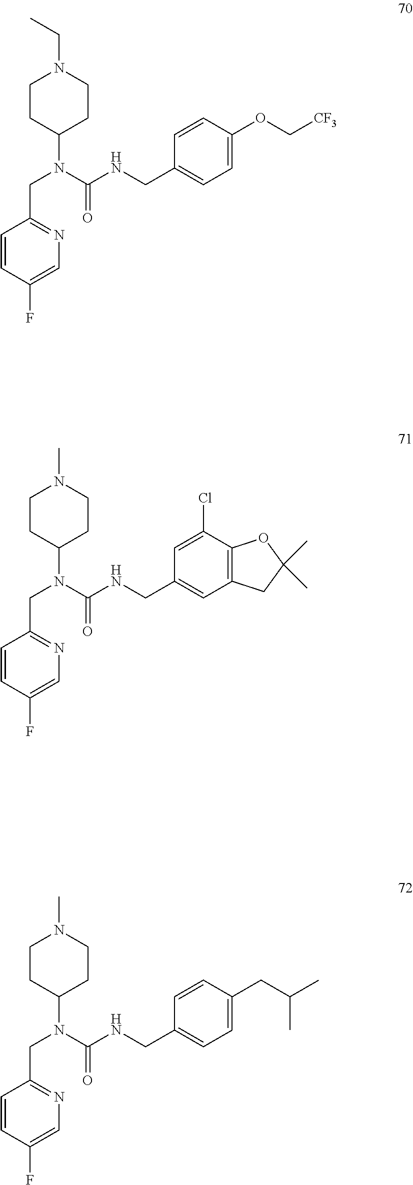 [SMILES: CCN1CCC(N(Cc2ccc(F)cn2)C(=O)NCc2ccc(OCC(F)(F)F)cc2)CC1 ; CN1CCC(N(Cc2ccc(F)cn2)C(=O)NCc2cc(Cl)c3c(c2)CC(C)(C)O3)CC1 ; CC(C)Cc1ccc(CNC(=O)N(Cc2ccc(F)cn2)C2CCN(C)CC2)cc1]

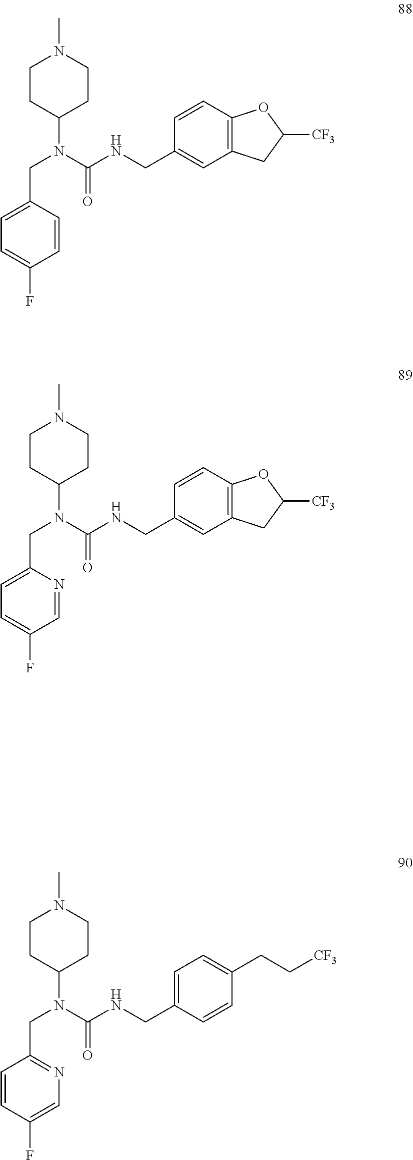 [SMILES: CN1CCC(N(Cc2ccc(F)cc2)C(=O)NCc2ccc3c(c2)CC(C(F)(F)F)O3)CC1 ; CN1CCC(N(Cc2ccc(F)cn2)C(=O)NCc2ccc3c(c2)CC(C(F)(F)F)O3)CC1 ; CN1CCC(N(Cc2ccc(F)cn2)C(=O)NCc2ccc(CCC(F)(F)F)cc2)CC1]

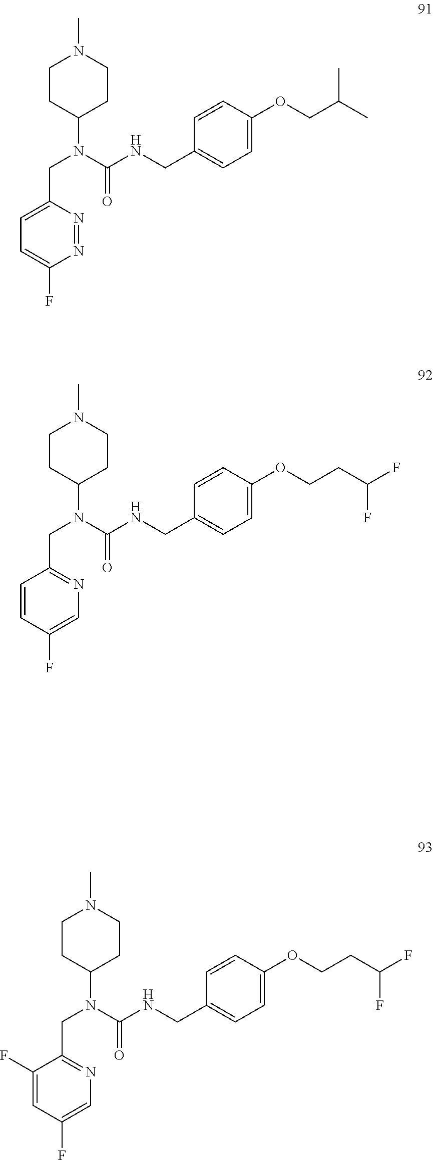 [SMILES: CC(C)COc1ccc(CNC(=O)N(Cc2ccc(F)nn2)C2CCN(C)CC2)cc1 ; CN1CCC(N(Cc2ccc(F)cn2)C(=O)NCc2ccc(OCCC(F)F)cc2)CC1 ; CN1CCC(N(Cc2ncc(F)cc2F)C(=O)NCc2ccc(OCCC(F)F)cc2)CC1]

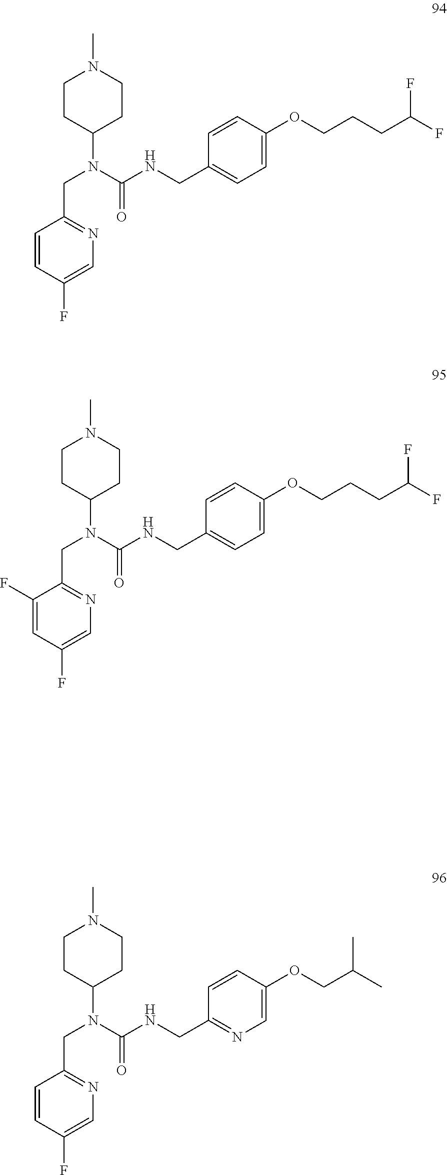 [SMILES: CN1CCC(N(Cc2ccc(F)cn2)C(=O)NCc2ccc(OCCCC(F)F)cc2)CC1 ; CN1CCC(N(Cc2ncc(F)cc2F)C(=O)NCc2ccc(OCCCC(F)F)cc2)CC1 ; CC(C)COc1ccc(CNC(=O)N(Cc2ccc(F)cn2)C2CCN(C)CC2)nc1]

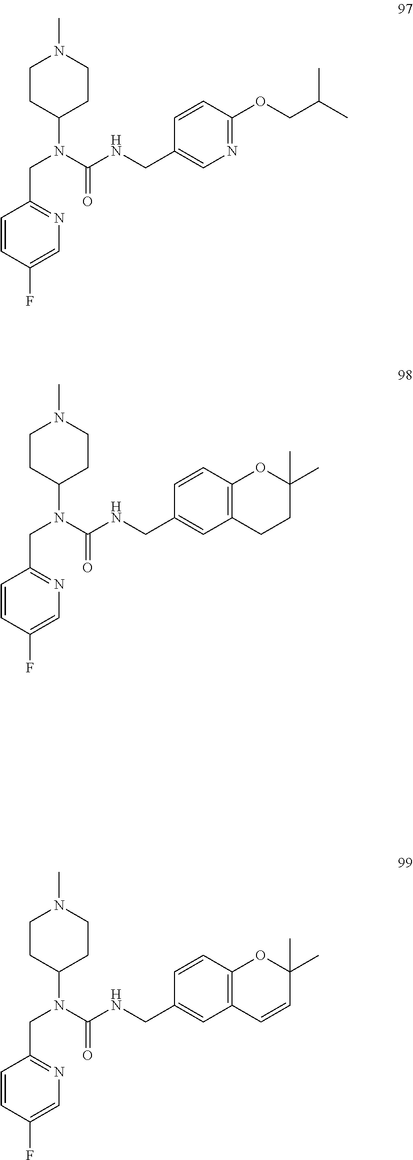 [SMILES: CC(C)COc1ccc(CNC(=O)N(Cc2ccc(F)cn2)C2CCN(C)CC2)cn1 ; CN1CCC(N(Cc2ccc(F)cn2)C(=O)NCc2ccc3c(c2)CCC(C)(C)O3)CC1 ; CN1CCC(N(Cc2ccc(F)cn2)C(=O)NCc2ccc3c(c2)C=CC(C)(C)O3)CC1]

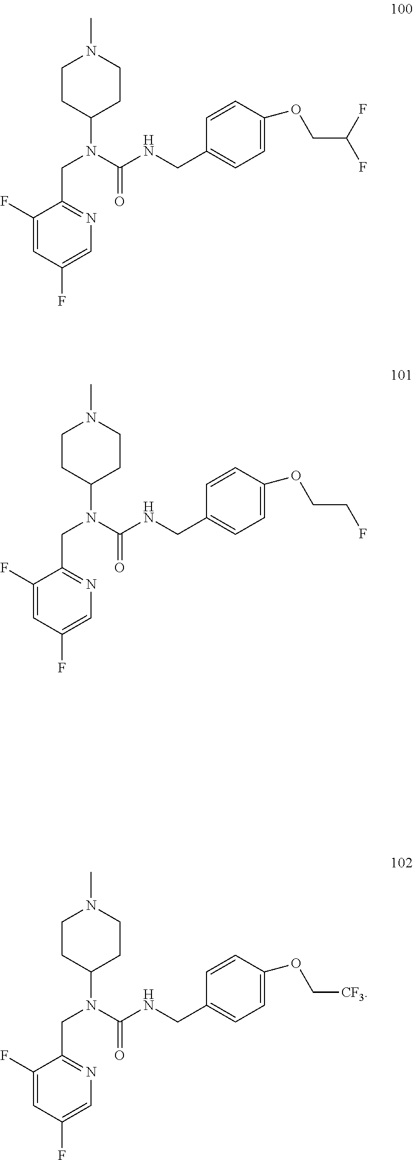 [SMILES: CN1CCC(N(Cc2ncc(F)cc2F)C(=O)NCc2ccc(OCC(F)F)cc2)CC1 ; CN1CCC(N(Cc2ncc(F)cc2F)C(=O)NCc2ccc(OCCF)cc2)CC1 ; CN1CCC(N(Cc2ncc(F)cc2F)C(=O)NCc2ccc(OCC(F)(F)F)cc2)CC1]

9. A pharmaceutical composition, comprising the compound or the pharmaceutically acceptable salt thereof according to claim 1 and a pharmaceutically acceptable carrier.

10. A method for treating 5-HT receptor-related diseases, the method comprising administering a therapeutically effective amount of the pharmaceutical composition according to claim 9 to a subject in need thereof, wherein preferably the 5-HT receptor-related diseases comprise: schizophrenia, psychosis, schizoaffective disorder, mania, psychotic depression, affective disorder, dementia, anxiety disorder, sleep disorder, dysorexia, bipolar disorder, psychosis secondary to hypertension, migraine, hypertension, thrombosis, vasospasm, ischemia, motor tics, depression, major depressive disorder, anxiety, sleep disturbance, eating disorder, non-motor symptoms caused by Parkinson's disease, delusion, illusion, cognitive disorder, dementia-related mental diseases, negative symptoms of schizophrenia, Parkinson's disease, Huntington's disease, Alzheimer's disease, spinocerebellar ataxia, Tourette's syndrome, Friedreich's ataxia, Machado-Joseph disease, Lewy body dementia, dyskinesia, dysmyotonia, myoclonus, tremor or progressive supranuclear paralysis and frontotemporal dementia.
